SEQUENCE LISTING (389 sequences)

SEQ ID NO: 1 - protein, 16 aa, Artificial Sequence
  Synthetic
CARQEDTAMVYYFDYW

SEQ ID NO: 2 - protein, 9 aa, Artificial Sequence
  Synthetic
NLFLFLFAV

SEQ ID NO: 3 - protein, 9 aa, Artificial Sequence
  Synthetic
NLFLFLFAV

SEQ ID NO: 4 - protein, 9 aa, Artificial Sequence
  Synthetic
FLWSVFMLI

SEQ ID NO: 5 - protein, 9 aa, Artificial Sequence
  Synthetic
FLWSVFMLI

SEQ ID NO: 6 - protein, 10 aa, Artificial Sequence
  Synthetic
FLWYSTSESH

SEQ ID NO: 7 - protein, 10 aa, Artificial Sequence
  Synthetic
KITLFVIVPV

SEQ ID NO: 8 - protein, 9 aa, Artificial Sequence
  Synthetic
TLFVIVPVL

SEQ ID NO: 9 - protein, 10 aa, Artificial Sequence
  Synthetic
KITLFVIVPV

SEQ ID NO: 10 - protein, 9 aa, Artificial Sequence
  Synthetic
TLFVIVPVL

SEQ ID NO: 11 - protein, 10 aa, Artificial Sequence
  Synthetic
FLQYSTSECH

SEQ ID NO: 12 - protein, 9 aa, Artificial Sequence
  Synthetic
FLIVLSVAL

SEQ ID NO: 13 - protein, 9 aa, Artificial Sequence
  Synthetic
FLIVLSVAL

SEQ ID NO: 14 - protein, 9 aa, Artificial Sequence
  Synthetic
KLQVFLIVL

SEQ ID NO: 15 - protein, 9 aa, Artificial Sequence
  Synthetic
KLQVFLIVL

SEQ ID NO: 16 - protein, 10 aa, Artificial Sequence
  Synthetic
VMNILLQYVV

SEQ ID NO: 17 - protein, 10 aa, Artificial Sequence
  Synthetic
VMNILLQYVV

SEQ ID NO: 18 - protein, 9 aa, Artificial Sequence
  Synthetic
KYCLITIFL

SEQ ID NO: 19 - protein, 10 aa, Artificial Sequence
  Synthetic
RMMEYGTTMV

SEQ ID NO: 20 - protein, 10 aa, Artificial Sequence
  Synthetic
RMMEYGTTMV

SEQ ID NO: 21 - protein, 9 aa, Artificial Sequence
  Synthetic
VIVMLTPLV

SEQ ID NO: 22 - protein, 9 aa, Artificial Sequence
  Synthetic
VIVMLTPLV

SEQ ID NO: 23 - protein, 9 aa, Artificial Sequence
  Synthetic
KYNIANVFL

SEQ ID NO: 24 - protein, 9 aa, Artificial Sequence
  Synthetic
ALYLVCGER

SEQ ID NO: 25 - protein, 10 aa, Artificial Sequence
  Synthetic
LYLVCGERGF

SEQ ID NO: 26 - protein, 9 aa, Artificial Sequence
  Synthetic
LYLVCGERG

SEQ ID NO: 27 - protein, 9 aa, Artificial Sequence
  Synthetic
LYLVCGERI

SEQ ID NO: 28 - protein, 9 aa, Artificial Sequence
  Synthetic
ALYLVCGER

SEQ ID NO: 29 - protein, 10 aa, Artificial Sequence
  Synthetic
LYLVCGERGF

SEQ ID NO: 30 - protein, 15 aa, Artificial Sequence
  Synthetic
SHLVEALYLVCGERG

SEQ ID NO: 31 - protein, 9 aa, Artificial Sequence
  Synthetic
FLFAVGFYL

SEQ ID NO: 32 - protein, 9 aa, Artificial Sequence
  Synthetic
FLFAVGFYL

SEQ ID NO: 33 - protein, 9 aa, Artificial Sequence
  Synthetic
KYQAVTTTL

SEQ ID NO: 34 - protein, 10 aa, Artificial Sequence
  Synthetic
GIVEQCCTSI

SEQ ID NO: 35 - protein, 9 aa, Artificial Sequence
  Synthetic
IVEQCCTSI

SEQ ID NO: 36 - protein, 10 aa, Artificial Sequence
  Synthetic
GIVEQCCTSI

SEQ ID NO: 37 - protein, 9 aa, Artificial Sequence
  Synthetic
IVEQCCTSI

SEQ ID NO: 38 - protein, 9 aa, Artificial Sequence
  Synthetic
RLLCALTSL

SEQ ID NO: 39 - protein, 9 aa, Artificial Sequence
  Synthetic
RLLCALTSL

SEQ ID NO: 40 - protein, 15 aa, Artificial Sequence
  Synthetic
SHLVEELYLVAGEEG

SEQ ID NO: 41 - protein, 9 aa, Artificial Sequence
  Synthetic
VLFGLGFAI

SEQ ID NO: 42 - protein, 9 aa, Artificial Sequence
  Synthetic
VLFGLGFAI

SEQ ID NO: 43 - protein, 9 aa, Artificial Sequence
  Synthetic
VLFGLGFAI

SEQ ID NO: 44 - protein, 10 aa, Artificial Sequence
  Synthetic
IILVEALYLV

SEQ ID NO: 45 - protein, 9 aa, Artificial Sequence
  Synthetic
FLYGALLLA

SEQ ID NO: 46 - protein, 9 aa, Artificial Sequence
  Synthetic
FLYGALLLA

SEQ ID NO: 47 - protein, 9 aa, Artificial Sequence
  Synthetic
LNIDLLWSV

SEQ ID NO: 48 - protein, 9 aa, Artificial Sequence
  Synthetic
LNIDLLWSV

SEQ ID NO: 49 - protein, 9 aa, Artificial Sequence
  Synthetic
LNIDLLWSV

SEQ ID NO: 50 - protein, 9 aa, Artificial Sequence
  Synthetic
FQDENYLYL

SEQ ID NO: 51 - protein, 9 aa, Artificial Sequence
  Synthetic
HLVEALYLV

SEQ ID NO: 52 - protein, 9 aa, Artificial Sequence
  Synthetic
HLVEALYLV

SEQ ID NO: 53 - protein, 9 aa, Artificial Sequence
  Synthetic
SLYQLENYC

SEQ ID NO: 54 - protein, 9 aa, Artificial Sequence
  Synthetic
SLYQLENYC

SEQ ID NO: 55 - protein, 10 aa, Artificial Sequence
  Synthetic
HLCGSHLVEA

SEQ ID NO: 56 - protein, 10 aa, Artificial Sequence
  Synthetic
HLCGSHLVEA

SEQ ID NO: 57 - protein, 10 aa, Artificial Sequence
  Synthetic
FLEYSTSECH

SEQ ID NO: 58 - protein, 10 aa, Artificial Sequence
  Synthetic
RLAGQFLEEL

SEQ ID NO: 59 - protein, 10 aa, Artificial Sequence
  Synthetic
RLAGQFLEEL

SEQ ID NO: 60 - protein, 9 aa, Artificial Sequence
  Synthetic
VLFSSDFRI

SEQ ID NO: 61 - protein, 9 aa, Artificial Sequence
  Synthetic
VLFSSDFRI

SEQ ID NO: 62 - protein, 9 aa, Artificial Sequence
  Synthetic
SLAAGVKLL

SEQ ID NO: 63 - protein, 9 aa, Artificial Sequence
  Synthetic
SLAAGVKLL

SEQ ID NO: 64 - protein, 9 aa, Artificial Sequence
  Synthetic
FLEYDTYEC

SEQ ID NO: 65 - protein, 10 aa, Artificial Sequence
  Synthetic
NLAQTDLATV

SEQ ID NO: 66 - protein, 10 aa, Artificial Sequence
  Synthetic
NLAQTDLATV

SEQ ID NO: 67 - protein, 10 aa, Artificial Sequence
  Synthetic
FLKYSTSECH

SEQ ID NO: 68 - protein, 9 aa, Artificial Sequence
  Synthetic
SLLLELEEV

SEQ ID NO: 69 - protein, 9 aa, Artificial Sequence
  Synthetic
SLLLELEEV

SEQ ID NO: 70 - protein, 9 aa, Artificial Sequence
  Synthetic
YLLLRVLNI

SEQ ID NO: 71 - protein, 9 aa, Artificial Sequence
  Synthetic
YLLLRVLNI

SEQ ID NO: 72 - protein, 9 aa, Artificial Sequence
  Synthetic
VYLKTNVFL

SEQ ID NO: 73 - protein, 9 aa, Artificial Sequence
  Synthetic
VYLKTNLFL

SEQ ID NO: 74 - protein, 10 aa, Artificial Sequence
  Synthetic
FLKEPMGECH

SEQ ID NO: 75 - protein, 9 aa, Artificial Sequence
  Synthetic
SLSPLQAEL

SEQ ID NO: 76 - protein, 9 aa, Artificial Sequence
  Synthetic
SLSPLQAEL

SEQ ID NO: 77 - protein, 10 aa, Artificial Sequence
  Synthetic
FLQLLRRECH

SEQ ID NO: 78 - protein, 10 aa, Artificial Sequence
  Synthetic
FLEQVKHKYY

SEQ ID NO: 79 - protein, 9 aa, Artificial Sequence
  Synthetic
ALWMRLLPL

SEQ ID NO: 80 - protein, 9 aa, Artificial Sequence
  Synthetic
LWMRLLPLL

SEQ ID NO: 81 - protein, 9 aa, Artificial Sequence
  Synthetic
ALWMRLLPL

SEQ ID NO: 82 - protein, 9 aa, Artificial Sequence
  Synthetic
LWMRLLPLL

SEQ ID NO: 83 - protein, 9 aa, Artificial Sequence
  Synthetic
SLQPLALEG

SEQ ID NO: 84 - protein, 9 aa, Artificial Sequence
  Synthetic
SLQPLALEG

SEQ ID NO: 85 - protein, 9 aa, Artificial Sequence
  Synthetic
LMWAKIGPV

SEQ ID NO: 86 - protein, 9 aa, Artificial Sequence
  Synthetic
LMWAKIGPV

SEQ ID NO: 87 - protein, 10 aa, Artificial Sequence
  Synthetic
RTFDPHFLRV

SEQ ID NO: 88 - protein, 10 aa, Artificial Sequence
  Synthetic
RTFDPHFLRV

SEQ ID NO: 89 - protein, 9 aa, Artificial Sequence
  Synthetic
KYNKANAFL

SEQ ID NO: 90 - protein, 9 aa, Artificial Sequence
  Synthetic
KYNKANVFL

SEQ ID NO: 91 - protein, 9 aa, Artificial Sequence
  Synthetic
KVEDPFYWV

SEQ ID NO: 92 - protein, 9 aa, Artificial Sequence
  Synthetic
KVEDPFYWV

SEQ ID NO: 93 - protein, 11 aa, Artificial Sequence
  Synthetic
TPKTRREAEDL

SEQ ID NO: 94 - protein, 11 aa, Artificial Sequence
  Synthetic
TPKTRREAEDL

SEQ ID NO: 95 - protein, 9 aa, Artificial Sequence
  Synthetic
FLEYRTKEC

SEQ ID NO: 96 - protein, 9 aa, Artificial Sequence
  Synthetic
ALEGSLQKR

SEQ ID NO: 97 - protein, 9 aa, Artificial Sequence
  Synthetic
ALEGSLQKR

SEQ ID NO: 98 - protein, 9 aa, Artificial Sequence
  Synthetic
FLRVPCWKI

SEQ ID NO: 99 - protein, 9 aa, Artificial Sequence
  Synthetic
FLRVPCWKI

SEQ ID NO: 100 - protein, 9 aa, Artificial Sequence
  Synthetic
FYTPKTRRE

SEQ ID NO: 101 - protein, 9 aa, Artificial Sequence
  Synthetic
FYTPKTRRE

SEQ ID NO: 102 - protein, 10 aa, Artificial Sequence
  Synthetic
SLQKRGIVEQ

SEQ ID NO: 103 - protein, 10 aa, Artificial Sequence
  Synthetic
SLQKRGIVEQ

SEQ ID NO: 104 - protein, 9 aa, Artificial Sequence
  Synthetic
ERGFFYTPK

SEQ ID NO: 105 - protein, 9 aa, Artificial Sequence
  Synthetic
ERGFFYTPK

SEQ ID NO: 106 - protein, 9 aa, Artificial Sequence
  Synthetic
LVCGERGFF

SEQ ID NO: 107 - protein, 10 aa, Artificial Sequence
  Synthetic
VCGERGFFYT

SEQ ID NO: 108 - protein, 8 aa, Artificial Sequence
  Synthetic
GERGFFYT

SEQ ID NO: 109 - protein, 9 aa, Artificial Sequence
  Synthetic
LVCGERGFF

SEQ ID NO: 110 - protein, 10 aa, Artificial Sequence
  Synthetic
VCGERGFFYT

SEQ ID NO: 111 - protein, 8 aa, Artificial Sequence
  Synthetic
GERGFFYT

SEQ ID NO: 112 - protein, 9 aa, Artificial Sequence
  Synthetic
QLARQQVHV

SEQ ID NO: 113 - protein, 9 aa, Artificial Sequence
  Synthetic
QLARQQVHV

SEQ ID NO: 114 - protein, 14 aa, Artificial Sequence
  Synthetic
KALVQNDTLLQVKG

SEQ ID NO: 115 - protein, 17 aa, Artificial Sequence
  Synthetic
KAMDIMNSFVNDIFERI

SEQ ID NO: 116 - protein, 17 aa, Artificial Sequence
  Synthetic
KAMGIMKSFVNDIFERI

SEQ ID NO: 117 - protein, 17 aa, Artificial Sequence
  Synthetic
KAMGNMNSFVNDIFERI

SEQ ID NO: 118 - protein, 17 aa, Artificial Sequence
  Synthetic
KAMSIMNSFVNDLFERL

SEQ ID NO: 119 - protein, 14 aa, Artificial Sequence
  Synthetic
KASGPPVSELITKA

SEQ ID NO: 120 - protein, 15 aa, Artificial Sequence
  Synthetic
KDAFLGSFLYEYSRR

SEQ ID NO: 121 - protein, 15 aa, Artificial Sequence
  Synthetic
KDDPHACYSTVFDKL

SEQ ID NO: 122 - protein, 18 aa, Artificial Sequence
  Synthetic
KEFFQSAIKLVDFQDAKA

SEQ ID NO: 123 - protein, 11 aa, Artificial Sequence
  Synthetic
KESYSVYVYKV

SEQ ID NO: 124 - protein, 23 aa, Artificial Sequence
  Synthetic
KGLVLIAFSQYLQQCPFDEHVKL

SEQ ID NO: 125 - protein, 13 aa, Artificial Sequence
  Synthetic
KHLVDEPQNLIKQ

SEQ ID NO: 126 - protein, 15 aa, Artificial Sequence
  Synthetic
KHPDSSVNFAEFSKK

SEQ ID NO: 127 - protein, 12 aa, Artificial Sequence
  Synthetic
KKQTALVELLKH

SEQ ID NO: 128 - protein, 17 aa, Artificial Sequence
  Synthetic
KKVPEVSTPTLVEVSRN

SEQ ID NO: 129 - protein, 18 aa, Artificial Sequence
  Synthetic
KLFTFHADICTLPDTEKQ

SEQ ID NO: 130 - protein, 15 aa, Artificial Sequence
  Synthetic
KLGEYGFQNALIVRY

SEQ ID NO: 131 - protein, 15 aa, Artificial Sequence
  Synthetic
KLKPDPNTLCDEFKA

SEQ ID NO: 132 - protein, 12 aa, Artificial Sequence
  Synthetic
KLVNELTEFAKT

SEQ ID NO: 133 - protein, 11 aa, Artificial Sequence
  Synthetic
KLVVSTQTALA

SEQ ID NO: 134 - protein, 11 aa, Artificial Sequence
  Synthetic
KQTALVELLKH

SEQ ID NO: 135 - protein, 14 aa, Artificial Sequence
  Synthetic
KSLHTLFGDELCKV

SEQ ID NO: 136 - protein, 18 aa, Artificial Sequence
  Synthetic
KTITLEVEPSDTIENVKA

SEQ ID NO: 137 - protein, 14 aa, Artificial Sequence
  Synthetic
KTVMENFVAFVDKC

SEQ ID NO: 138 - protein, 31 aa, Artificial Sequence
  Synthetic
KTVMENFVAFVDKCCAADDKEACFAVEGPKL

SEQ ID NO: 139 - protein, 14 aa, Artificial Sequence
  Synthetic
KTVTAMDVVYALKR

SEQ ID NO: 140 - protein, 10 aa, Artificial Sequence
  Synthetic
KVFLENVIRD

SEQ ID NO: 141 - protein, 16 aa, Artificial Sequence
  Synthetic
KVPEVSTPTLVEVSRN

SEQ ID NO: 142 - protein, 9 aa, Artificial Sequence
  Synthetic
KYLYEIARR

SEQ ID NO: 143 - protein, 15 aa, Artificial Sequence
  Synthetic
MGIMNSFVNDIFERI

SEQ ID NO: 144 - protein, 11 aa, Artificial Sequence
  Synthetic
RAGLQFPVGRV

SEQ ID NO: 145 - protein, 14 aa, Artificial Sequence
  Synthetic
RDNIQGITKPAIRR

SEQ ID NO: 146 - protein, 13 aa, Artificial Sequence
  Synthetic
REIAQDFKTDLRF

SEQ ID NO: 147 - protein, 34 aa, Artificial Sequence
  Synthetic
RFQSAAIGALQEASEAYLVGLFEDTNLCAIHAKR

SEQ ID NO: 148 - protein, 12 aa, Artificial Sequence
  Synthetic
RILGLIYEETRR

SEQ ID NO: 149 - protein, 12 aa, Artificial Sequence
  Synthetic
RISGLIYEETRG

SEQ ID NO: 150 - protein, 12 aa, Artificial Sequence
  Synthetic
RISGLIYKETRR

SEQ ID NO: 151 - protein, 12 aa, Artificial Sequence
  Synthetic
RKENHSVYVYKV

SEQ ID NO: 152 - protein, 11 aa, Artificial Sequence
  Synthetic
RLLLPGELAKH

SEQ ID NO: 153 - protein, 13 aa, Artificial Sequence
  Synthetic
RNDEELNKLLGKV

SEQ ID NO: 154 - protein, 18 aa, Artificial Sequence
  Synthetic
RNECFLSHKDDSPDLPKL

SEQ ID NO: 155 - protein, 18 aa, Artificial Sequence
  Synthetic
RRPCFSALTPDETYVPKA

SEQ ID NO: 156 - protein, 8 aa, Artificial Sequence
  Synthetic
RTLYGFGG

SEQ ID NO: 157 - protein, 17 aa, Artificial Sequence
  Synthetic
RTSKLQNEIDVSSREKS

SEQ ID NO: 158 - protein, 21 aa, Artificial Sequence
  Synthetic
RVTIAQGGVLPNIQAVLLPKK

SEQ ID NO: 159 - protein, 9 aa, Artificial Sequence
  Synthetic
LPDTEKQKL

SEQ ID NO: 160 - protein, 9 aa, Artificial Sequence
  Synthetic
YSTVFDKLK

SEQ ID NO: 161 - protein, 9 aa, Artificial Sequence
  Synthetic
ITLEVEPSD

SEQ ID NO: 162 - protein, 9 aa, Artificial Sequence
  Synthetic
LVQNDTLLQ

SEQ ID NO: 163 - protein, 9 aa, Artificial Sequence
  Synthetic
IKAMGIMKS

SEQ ID NO: 164 - protein, 9 aa, Artificial Sequence
  Synthetic
IKAMSIMNS

SEQ ID NO: 165 - protein, 9 aa, Artificial Sequence
  Synthetic
YVYKVRLLL

SEQ ID NO: 166 - protein, 9 aa, Artificial Sequence
  Synthetic
IKAMGNMNS

SEQ ID NO: 167 - protein, 9 aa, Artificial Sequence
  Synthetic
VRLLLPGEL

SEQ ID NO: 168 - protein, 9 aa, Artificial Sequence
  Synthetic
VVYALKRKV

SEQ ID NO: 169 - protein, 9 aa, Artificial Sequence
  Synthetic
YEIARRMGI

SEQ ID NO: 170 - protein, 9 aa, Artificial Sequence
  Synthetic
FRFQSAAIG

SEQ ID NO: 171 - protein, 9 aa, Artificial Sequence
  Synthetic
VVSTQTALA

SEQ ID NO: 172 - protein, 9 aa, Artificial Sequence
  Synthetic
IMNSFVNDI

SEQ ID NO: 173 - protein, 9 aa, Artificial Sequence
  Synthetic
ICTLPDTEK

SEQ ID NO: 174 - protein, 9 aa, Artificial Sequence
  Synthetic
MGIMKSFVN

SEQ ID NO: 175 - protein, 9 aa, Artificial Sequence
  Synthetic
MGIMNSFVN

SEQ ID NO: 176 - protein, 9 aa, Artificial Sequence
  Synthetic
LVELLKHKS

SEQ ID NO: 177 - protein, 9 aa, Artificial Sequence
  Synthetic
FERIKAMGI

SEQ ID NO: 178 - protein, 9 aa, Artificial Sequence
  Synthetic
FERIKAMSI

SEQ ID NO: 179 - protein, 9 aa, Artificial Sequence
  Synthetic
VLIAFSQYL

SEQ ID NO: 180 - protein, 9 aa, Artificial Sequence
  Synthetic
IMNSFVNDL

SEQ ID NO: 181 - protein, 9 aa, Artificial Sequence
  Synthetic
IMKSFVNDI

SEQ ID NO: 182 - protein, 9 aa, Artificial Sequence
  Synthetic
IQGITKPAI

SEQ ID NO: 183 - protein, 9 aa, Artificial Sequence
  Synthetic
VYVYKVRLL

SEQ ID NO: 184 - protein, 9 aa, Artificial Sequence
  Synthetic
YVYKVKGLV

SEQ ID NO: 185 - protein, 9 aa, Artificial Sequence
  Synthetic
LIYKETRRR

SEQ ID NO: 186 - protein, 9 aa, Artificial Sequence
  Synthetic
VKGLVLIAF

SEQ ID NO: 187 - protein, 9 aa, Artificial Sequence
  Synthetic
IRRREIAQD

SEQ ID NO: 188 - protein, 9 aa, Artificial Sequence
  Synthetic
VYVYKVKGL

SEQ ID NO: 189 - protein, 9 aa, Artificial Sequence
  Synthetic
VTAMDVVYA

SEQ ID NO: 190 - protein, 9 aa, Artificial Sequence
  Synthetic
YGFQNALIV

SEQ ID NO: 191 - protein, 9 aa, Artificial Sequence
  Synthetic
LVNELTEFA

SEQ ID NO: 192 - protein, 9 aa, Artificial Sequence
  Synthetic
VRYKLKPDP

SEQ ID NO: 193 - protein, 9 aa, Artificial Sequence
  Synthetic
LKTVTAMDV

SEQ ID NO: 194 - protein, 9 aa, Artificial Sequence
  Synthetic
FQNALIVRY

SEQ ID NO: 195 - protein, 9 aa, Artificial Sequence
  Synthetic
MSIMNSFVN

SEQ ID NO: 196 - protein, 9 aa, Artificial Sequence
  Synthetic
VKAKTVMEN

SEQ ID NO: 197 - protein, 9 aa, Artificial Sequence
  Synthetic
FKAKLVNEL

SEQ ID NO: 198 - protein, 9 aa, Artificial Sequence
  Synthetic
LRFRFQSAA

SEQ ID NO: 199 - protein, 9 aa, Artificial Sequence
  Synthetic
LVLIAFSQY

SEQ ID NO: 200 - protein, 9 aa, Artificial Sequence
  Synthetic
LKASGPPVS

SEQ ID NO: 201 - protein, 9 aa, Artificial Sequence
  Synthetic
VIRDKVPEV

SEQ ID NO: 202 - protein, 9 aa, Artificial Sequence
  Synthetic
VQNDTLLQV

SEQ ID NO: 203 - protein, 9 aa, Artificial Sequence
  Synthetic
MGNMNSFVN

SEQ ID NO: 204 - protein, 9 aa, Artificial Sequence
  Synthetic
YVPKARTLY

SEQ ID NO: 205 - protein, 9 aa, Artificial Sequence
  Synthetic
FQSAIKLVD

SEQ ID NO: 206 - protein, 9 aa, Artificial Sequence
  Synthetic
LYGFGGRTS

SEQ ID NO: 207 - protein, 9 aa, Artificial Sequence
  Synthetic
YKVKGLVLI

SEQ ID NO: 208 - protein, 9 aa, Artificial Sequence
  Synthetic
LVELLKHKK

SEQ ID NO: 209 - protein, 9 aa, Artificial Sequence
  Synthetic
LKHKKVPEV

SEQ ID NO: 210 - protein, 9 aa, Artificial Sequence
  Synthetic
LLKHKSLHT

SEQ ID NO: 211 - protein, 9 aa, Artificial Sequence
  Synthetic
YKVRLLLPG

SEQ ID NO: 212 - protein, 9 aa, Artificial Sequence
  Synthetic
VRNECFLSH

SEQ ID NO: 213 - protein, 9 aa, Artificial Sequence
  Synthetic
IVRYKLKPD

SEQ ID NO: 214 - protein, 9 aa, Artificial Sequence
  Synthetic
LIVRYKLKP

SEQ ID NO: 215 - protein, 9 aa, Artificial Sequence
  Synthetic
LLGKVRNEC

SEQ ID NO: 216 - protein, 9 aa, Artificial Sequence
  Synthetic
FERIKAMGN

SEQ ID NO: 217 - protein, 9 aa, Artificial Sequence
  Synthetic
VAFVDKCCA

SEQ ID NO: 218 - protein, 9 aa, Artificial Sequence
  Synthetic
LIYEETRRR

SEQ ID NO: 219 - protein, 9 aa, Artificial Sequence
  Synthetic
LIYEETRGR

SEQ ID NO: 220 - protein, 9 aa, Artificial Sequence
  Synthetic
VYALKRKVF

SEQ ID NO: 221 - protein, 9 aa, Artificial Sequence
  Synthetic
YLYEIARRM

SEQ ID NO: 222 - protein, 9 aa, Artificial Sequence
  Synthetic
LVVSTQTAL

SEQ ID NO: 223 - protein, 9 aa, Artificial Sequence
  Synthetic
VFLENVIRD

SEQ ID NO: 224 - protein, 9 aa, Artificial Sequence
  Synthetic
LVEVSRNKL

SEQ ID NO: 225 - protein, 9 aa, Artificial Sequence
  Synthetic
LIAFSQYLQ

SEQ ID NO: 226 - protein, 9 aa, Artificial Sequence
  Synthetic
IRDKVPEVS

SEQ ID NO: 227 - protein, 9 aa, Artificial Sequence
  Synthetic
LCKVKTITL

SEQ ID NO: 228 - protein, 9 aa, Artificial Sequence
  Synthetic
LIKQKHPDS

SEQ ID NO: 229 - protein, 9 aa, Artificial Sequence
  Synthetic
FERIRAGLQ

SEQ ID NO: 230 - protein, 9 aa, Artificial Sequence
  Synthetic
FQSAAIGAL

SEQ ID NO: 231 - protein, 9 aa, Artificial Sequence
  Synthetic
LVEVSRNKY

SEQ ID NO: 232 - protein, 9 aa, Artificial Sequence
  Synthetic
VKLKHLVDE

SEQ ID NO: 233 - protein, 9 aa, Artificial Sequence
  Synthetic
VYKVKGLVL

SEQ ID NO: 234 - protein, 9 aa, Artificial Sequence
  Synthetic
YALKRKVFL

SEQ ID NO: 235 - protein, 9 aa, Artificial Sequence
  Synthetic
VELLKHKKV

SEQ ID NO: 236 - protein, 9 aa, Artificial Sequence
  Synthetic
LQVKGKAMD

SEQ ID NO: 237 - protein, 9 aa, Artificial Sequence
  Synthetic
LKHKSLHTL

SEQ ID NO: 238 - protein, 9 aa, Artificial Sequence
  Synthetic
VELLKHKSL

SEQ ID NO: 239 - protein, 9 aa, Artificial Sequence
  Synthetic
VPKARTLYG

SEQ ID NO: 240 - protein, 9 aa, Artificial Sequence
  Synthetic
FKTDLRFRF

SEQ ID NO: 241 - protein, 9 aa, Artificial Sequence
  Synthetic
MDIMNSFVN

SEQ ID NO: 242 - protein, 9 aa, Artificial Sequence
  Synthetic
IKLVDFQDA

SEQ ID NO: 243 - protein, 9 aa, Artificial Sequence
  Synthetic
FVDKCKTVM

SEQ ID NO: 244 - protein, 9 aa, Artificial Sequence
  Synthetic
IHAKRRILG

SEQ ID NO: 245 - protein, 9 aa, Artificial Sequence
  Synthetic
FLYEYSRRK

SEQ ID NO: 246 - protein, 9 aa, Artificial Sequence
  Synthetic
VMENFVAFV

SEQ ID NO: 247 - protein, 9 aa, Artificial Sequence
  Synthetic
YLVGLFEDT

SEQ ID NO: 248 - protein, 9 aa, Artificial Sequence
  Synthetic
VYKVRLLLP

SEQ ID NO: 249 - protein, 9 aa, Artificial Sequence
  Synthetic
YLQQCPFDE

SEQ ID NO: 250 - protein, 9 aa, Artificial Sequence
  Synthetic
IRAGLQFPV

SEQ ID NO: 251 - protein, 9 aa, Artificial Sequence
  Synthetic
LLKHKKVPE

SEQ ID NO: 252 - protein, 9 aa, Artificial Sequence
  Synthetic
IKQKHPDSS

SEQ ID NO: 253 - protein, 9 aa, Artificial Sequence
  Synthetic
VLPNIQAVL

SEQ ID NO: 254 - protein, 9 aa, Artificial Sequence
  Synthetic
VEPSDTIEN

SEQ ID NO: 255 - protein, 9 aa, Artificial Sequence
  Synthetic
FGGRTSKLQ

SEQ ID NO: 256 - protein, 9 aa, Artificial Sequence
  Synthetic
VAFVDKCKT

SEQ ID NO: 257 - protein, 9 aa, Artificial Sequence
  Synthetic
FFQSAIKLV

SEQ ID NO: 258 - protein, 9 aa, Artificial Sequence
  Synthetic
FQDAKAKES

SEQ ID NO: 259 - protein, 9 aa, Artificial Sequence
  Synthetic
IQAVLLPKK

SEQ ID NO: 260 - protein, 9 aa, Artificial Sequence
  Synthetic
LLQVKGKAM

SEQ ID NO: 261 - protein, 9 aa, Artificial Sequence
  Synthetic
IAFSQYLQQ

SEQ ID NO: 262 - protein, 9 aa, Artificial Sequence
  Synthetic
FLGSFLYEY

SEQ ID NO: 263 - protein, 9 aa, Artificial Sequence
  Synthetic
FVNDIFERI

SEQ ID NO: 264 - protein, 9 aa, Artificial Sequence
  Synthetic
VDEPQNLIK

SEQ ID NO: 265 - protein, 9 aa, Artificial Sequence
  Synthetic
LSHKDDSPD

SEQ ID NO: 266 - protein, 9 aa, Artificial Sequence
  Synthetic
FLSHKDDSP

SEQ ID NO: 267 - protein, 9 aa, Artificial Sequence
  Synthetic
LPNIQAVLL

SEQ ID NO: 268 - protein, 9 aa, Artificial Sequence
  Synthetic
LKRKVFLEN

SEQ ID NO: 269 - protein, 9 aa, Artificial Sequence
  Synthetic
LLPGELAKH

SEQ ID NO: 270 - protein, 9 aa, Artificial Sequence
  Synthetic
FVAFVDKCC

SEQ ID NO: 271 - protein, 9 aa, Artificial Sequence
  Synthetic
IFERIKAMS

SEQ ID NO: 272 - protein, 9 aa, Artificial Sequence
  Synthetic
IENVKAKTV

SEQ ID NO: 273 - protein, 9 aa, Artificial Sequence
  Synthetic
VSRNKLFTF

SEQ ID NO: 274 - protein, 9 aa, Artificial Sequence
  Synthetic
LKPDPNTLC

SEQ ID NO: 275 - protein, 9 aa, Artificial Sequence
  Synthetic
MENFVAFVD

SEQ ID NO: 276 - protein, 9 aa, Artificial Sequence
  Synthetic
YSRRKDDPH

SEQ ID NO: 277 - protein, 9 aa, Artificial Sequence
  Synthetic
LFGDELCKV

SEQ ID NO: 278 - protein, 9 aa, Artificial Sequence
  Synthetic
FERLKASGP

SEQ ID NO: 279 - protein, 9 aa, Artificial Sequence
  Synthetic
VSTQTALAK

SEQ ID NO: 280 - protein, 9 aa, Artificial Sequence
  Synthetic
FAKTKLVVS

SEQ ID NO: 281 - protein, 9 aa, Artificial Sequence
  Synthetic
VTIAQGGVL

SEQ ID NO: 282 - protein, 9 aa, Artificial Sequence
  Synthetic
LNKLLGKVR

SEQ ID NO: 283 - protein, 9 aa, Artificial Sequence
  Synthetic
LYEIARRMG

SEQ ID NO: 284 - protein, 9 aa, Artificial Sequence
  Synthetic
MKSFVNDIF

SEQ ID NO: 285 - protein, 9 aa, Artificial Sequence
  Synthetic
LFTFHADIC

SEQ ID NO: 286 - protein, 9 aa, Artificial Sequence
  Synthetic
LAKQTALVE

SEQ ID NO: 287 - protein, 9 aa, Artificial Sequence
  Synthetic
FVAFVDKCK

SEQ ID NO: 288 - protein, 9 aa, Artificial Sequence
  Synthetic
FVNDLFERL

SEQ ID NO: 289 - protein, 9 aa, Artificial Sequence
  Synthetic
VKTITLEVE

SEQ ID NO: 290 - protein, 9 aa, Artificial Sequence
  Synthetic
IAQGGVLPN

SEQ ID NO: 291 - protein, 9 aa, Artificial Sequence
  Synthetic
LRRPCFSAL

SEQ ID NO: 292 - protein, 9 aa, Artificial Sequence
  Synthetic
LGSFLYEYS

SEQ ID NO: 293 - protein, 9 aa, Artificial Sequence
  Synthetic
LCAIHAKRR

SEQ ID NO: 294 - protein, 9 aa, Artificial Sequence
  Synthetic
LPKLRRPCF

SEQ ID NO: 295 - protein, 9 aa, Artificial Sequence
  Synthetic
VEVSRNKLF

SEQ ID NO: 296 - protein, 9 aa, Artificial Sequence
  Synthetic
FLENVIRDK

SEQ ID NO: 297 - protein, 9 aa, Artificial Sequence
  Synthetic
IYKETRRRK

SEQ ID NO: 298 - protein, 9 aa, Artificial Sequence
  Synthetic
VEVSRNKYL

SEQ ID NO: 299 - protein, 9 aa, Artificial Sequence
  Synthetic
FVDKCCAAD

SEQ ID NO: 300 - protein, 9 aa, Artificial Sequence
  Synthetic
LFEDTNLCA

SEQ ID NO: 301 - protein, 9 aa, Artificial Sequence
  Synthetic
VNFAEFSKK

SEQ ID NO: 302 - protein, 9 aa, Artificial Sequence
  Synthetic
VGRVRDNIQ

SEQ ID NO: 303 - protein, 9 aa, Artificial Sequence
  Synthetic
MNSFVNDIF

SEQ ID NO: 304 - protein, 9 aa, Artificial Sequence
  Synthetic
MNSFVNDLF

SEQ ID NO: 305 - protein, 9 aa, Artificial Sequence
  Synthetic
LVDEPQNLI

SEQ ID NO: 306 - protein, 9 aa, Artificial Sequence
  Synthetic
FSKKKKQTA

SEQ ID NO: 307 - protein, 9 aa, Artificial Sequence
  Synthetic
YGFGGRTSK

SEQ ID NO: 308 - protein, 9 aa, Artificial Sequence
  Synthetic
LITKAKDAF

SEQ ID NO: 309 - protein, 9 aa, Artificial Sequence
  Synthetic
MDVVYALKR

SEQ ID NO: 310 - protein, 9 aa, Artificial Sequence
  Synthetic
LLLPGELAK

SEQ ID NO: 311 - protein, 9 aa, Artificial Sequence
  Synthetic
LQFPVGRVR

SEQ ID NO: 312 - protein, 9 aa, Artificial Sequence
  Synthetic
LKEFFQSAI

SEQ ID NO: 313 - protein, 9 aa, Artificial Sequence
  Synthetic
YEYSRRKDD

SEQ ID NO: 314 - protein, 9 aa, Artificial Sequence
  Synthetic
LTPDETYVP

SEQ ID NO: 315 - protein, 9 aa, Artificial Sequence
  Synthetic
LGKVRNECF

SEQ ID NO: 316 - protein, 9 aa, Artificial Sequence
  Synthetic
LKHLVDEPQ

SEQ ID NO: 317 - protein, 9 aa, Artificial Sequence
  Synthetic
LQNEIDVSS

SEQ ID NO: 318 - protein, 9 aa, Artificial Sequence
  Synthetic
LVDFQDAKA

SEQ ID NO: 319 - protein, 9 aa, Artificial Sequence
  Synthetic
FAVEGPKLK

SEQ ID NO: 320 - protein, 9 aa, Artificial Sequence
  Synthetic
VSELITKAK

SEQ ID NO: 321 - protein, 9 aa, Artificial Sequence
  Synthetic
IFERIRAGL

SEQ ID NO: 322 - protein, 9 aa, Artificial Sequence
  Synthetic
LENVIRDKV

SEQ ID NO: 323 - protein, 9 aa, Artificial Sequence
  Synthetic
VGLFEDTNL

SEQ ID NO: 324 - protein, 9 aa, Artificial Sequence
  Synthetic
VSSREKSRV

SEQ ID NO: 325 - protein, 9 aa, Artificial Sequence
  Synthetic
IYEETRRRI

SEQ ID NO: 326 - protein, 9 aa, Artificial Sequence
  Synthetic
IFERIKAMG

SEQ ID NO: 327 - protein, 9 aa, Artificial Sequence
  Synthetic
FGDELCKVK

SEQ ID NO: 328 - protein, 9 aa, Artificial Sequence
  Synthetic
LFERLKASG

SEQ ID NO: 329 - protein, 9 aa, Artificial Sequence
  Synthetic
IARRMGIMN

SEQ ID NO: 330 - protein, 9 aa, Artificial Sequence
  Synthetic
LGLIYEETR

SEQ ID NO: 331 - protein, 9 aa, Artificial Sequence
  Synthetic
ILGLIYEET

SEQ ID NO: 332 - protein, 9 aa, Artificial Sequence
  Synthetic
YEETRRRIS

SEQ ID NO: 333 - protein, 9 aa, Artificial Sequence
  Synthetic
IDVSSREKS

SEQ ID NO: 334 - protein, 9 aa, Artificial Sequence
  Synthetic
LHTLFGDEL

SEQ ID NO: 335 - protein, 9 aa, Artificial Sequence
  Synthetic
LVGLFEDTN

SEQ ID NO: 336 - protein, 9 aa, Artificial Sequence
  Synthetic
VKGKAMDIM

SEQ ID NO: 337 - protein, 9 aa, Artificial Sequence
  Synthetic
FPVGRVRDN

SEQ ID NO: 338 - protein, 9 aa, Artificial Sequence
  Synthetic
VSRNKYLYE

SEQ ID NO: 339 - protein, 9 aa, Artificial Sequence
  Synthetic
IAQDFKTDL

SEQ ID NO: 340 - protein, 9 aa, Artificial Sequence
  Synthetic
FHADICTLP

SEQ ID NO: 341 - protein, 9 aa, Artificial Sequence
  Synthetic
VRDNIQGIT

SEQ ID NO: 342 - protein, 9 aa, Artificial Sequence
  Synthetic
YKLKPDPNT

SEQ ID NO: 343 - protein, 9 aa, Artificial Sequence
  Synthetic
VDFQDAKAK

SEQ ID NO: 344 - protein, 9 aa, Artificial Sequence
  Synthetic
FAEFSKKKK

SEQ ID NO: 345 - protein, 9 aa, Artificial Sequence
  Synthetic
LYEYSRRKD

SEQ ID NO: 346 - protein, 9 aa, Artificial Sequence
  Synthetic
FDEHVKLKH

SEQ ID NO: 347 - protein, 9 aa, Artificial Sequence
  Synthetic
LTEFAKTKL

SEQ ID NO: 348 - protein, 9 aa, Artificial Sequence
  Synthetic
LQQCPFDEH

SEQ ID NO: 349 - protein, 9 aa, Artificial Sequence
  Synthetic
LEVEPSDTI

SEQ ID NO: 350 - protein, 9 aa, Artificial Sequence
  Synthetic
IGALQEASE

SEQ ID NO: 351 - protein, 9 aa, Artificial Sequence
  Synthetic
VDKCKTVME

SEQ ID NO: 352 - protein, 9 aa, Artificial Sequence
  Synthetic
VFDKLKEFF

SEQ ID NO: 353 - protein, 9 aa, Artificial Sequence
  Synthetic
FTFHADICT

SEQ ID NO: 354 - protein, 9 aa, Artificial Sequence
  Synthetic
VPEVSTPTL

SEQ ID NO: 355 - protein, 9 aa, Artificial Sequence
  Synthetic
FSALTPDET

SEQ ID NO: 356 - protein, 9 aa, Artificial Sequence
  Synthetic
ITKPAIRRR

SEQ ID NO: 357 - protein, 9 aa, Artificial Sequence
  Synthetic
YKETRRRKE

SEQ ID NO: 358 - protein, 9 aa, Artificial Sequence
  Synthetic
IYEETRGRI

SEQ ID NO: 359 - protein, 9 aa, Artificial Sequence
  Synthetic
VEGPKLKTV

SEQ ID NO: 360 - protein, 9 aa, Artificial Sequence
  Synthetic
FEDTNLCAI

SEQ ID NO: 361 - protein, 9 aa, Artificial Sequence
  Synthetic
VNELTEFAK

SEQ ID NO: 362 - protein, 9 aa, Artificial Sequence
  Synthetic
YSVYVYKVK

SEQ ID NO: 363 - protein, 9 aa, Artificial Sequence
  Synthetic
LQEASEAYL

SEQ ID NO: 364 - protein, 9 aa, Artificial Sequence
  Synthetic
ISGLIYKET

SEQ ID NO: 365 - protein, 9 aa, Artificial Sequence
  Synthetic
YEETRGRIS

SEQ ID NO: 366 - protein, 9 aa, Artificial Sequence
  Synthetic
FDKLKEFFQ

SEQ ID NO: 367 - protein, 9 aa, Artificial Sequence
  Synthetic
VSTPTLVEV

SEQ ID NO: 368 - protein, 9 aa, Artificial Sequence
  Synthetic
VNDLFERLK

SEQ ID NO: 369 - protein, 9 aa, Artificial Sequence
  Synthetic
LPGELAKHR

SEQ ID NO: 370 - protein, 9 aa, Artificial Sequence
  Synthetic
VNDIFERIK

SEQ ID NO: 371 - protein, 9 aa, Artificial Sequence
  Synthetic
FSQYLQQCP

SEQ ID NO: 372 - protein, 9 aa, Artificial Sequence
  Synthetic
ITKAKDAFL

SEQ ID NO: 373 - protein, 9 aa, Artificial Sequence
  Synthetic
LGEYGFQNA

SEQ ID NO: 374 - protein, 9 aa, Artificial Sequence
  Synthetic
LCDEFKAKL

SEQ ID NO: 375 - protein, 9 aa, Artificial Sequence
  Synthetic
VDKCCAADD

SEQ ID NO: 376 - protein, 9 aa, Artificial Sequence
  Synthetic
VNDIFERIR

SEQ ID NO: 377 - protein, 9 aa, Artificial Sequence
  Synthetic
ISGLIYEET

SEQ ID NO: 378 - protein, 9 aa, Artificial Sequence
  Synthetic
LAKHRNDEE

SEQ ID NO: 379 - protein, 23 aa, Artificial Sequence
  Synthetic
  SITE (5)..(5): Xaa can be any naturally occurring amino acid
  SITE (6)..(6): Xaa can be any naturally occurring amino acid
  SITE (7)..(7): Xaa can be any naturally occurring amino acid
  SITE (7)..(9): may be absent
  SITE (8)..(8): Xaa can be any naturally occurring amino acid
  SITE (9)..(9): Xaa can be any naturally occurring amino acid
  SITE (11)..(11): Xaa can be any naturally occurring amino acid
  SITE (12)..(12): Xaa can be any naturally occurring amino acid
  SITE (13)..(13): Xaa can be any naturally occurring amino acid
  SITE (13)..(15): may be absent
  SITE (14)..(14): Xaa can be any naturally occurring amino acid
  SITE (15)..(15): Xaa can be any naturally occurring amino acid
  SITE (18)..(18): Xaa can be any naturally occurring amino acid
  SITE (19)..(19): Xaa can be any naturally occurring amino acid
  SITE (19)..(19): may be absent
ANSGXXXXXIXXXXXLAXXGGQY

SEQ ID NO: 380 - protein, 17 aa, Artificial Sequence
  Synthetic
ANSGIRIMATLAIGGQY

SEQ ID NO: 381 - protein, 17 aa, Artificial Sequence
  Synthetic
ANSGFRIMAVLAIGGQY

SEQ ID NO: 382 - protein, 17 aa, Artificial Sequence
  Synthetic
ANSGIRIMAVLASGGQY

SEQ ID NO: 383 - protein, 9 aa, Artificial Sequence
  Synthetic
  SITE (1)..(1): Xaa can be any naturally occurring amino acid
  SITE (3)..(3): Xaa can be any naturally occurring amino acid
  SITE (3)..(4): at least one is Met
  SITE (4)..(4): Xaa can be any naturally occurring amino acid
  SITE (5)..(5): Xaa can be any naturally occurring amino acid
  SITE (6)..(6): Xaa can be any naturally occurring amino acid
  SITE (8)..(8): Xaa can be any naturally occurring amino acid
  SITE (9)..(9): Xaa can be any naturally occurring amino acid
XRXXXXLXX

SEQ ID NO: 384 - protein, 9 aa, Artificial Sequence
  Synthetic
  SITE (1)..(1): Xaa can be Ala, Phe, Met, Leu, Ile, Val, Pro, or Trp
  SITE (3)..(3): Xaa can be Ala, Phe, Met, Leu, Ile, Val, Pro, or Trp
  SITE (4)..(4): Xaa can be Ala, Phe, Met, Leu, Ile, Val, Pro, or Trp
  SITE (5)..(5): Xaa can be any naturally occurring amino acid
  SITE (6)..(6): Xaa can be Ala, Phe, Met, Leu, Ile, Val, Pro, or Trp
  SITE (8)..(8): Xaa can be any naturally occurring amino acid
  SITE (9)..(9): Xaa can be Ala, Cys, Thr, Ser, Gly, Asn, Gln, Tyr
XRXXXXLXX

SEQ ID NO: 385 - protein, 5 aa, Artificial Sequence
  Synthetic
LENLV

SEQ ID NO: 386 - protein, 5 aa, Artificial Sequence
  Synthetic
LNAAS

SEQ ID NO: 387 - protein, 23 aa, Artificial Sequence
  Synthetic
ANSGLENLVILNAASLAGTGGQY

SEQ ID NO: 388 - protein, 9 aa, Artificial Sequence
  Synthetic
  misc_feature (5)..(5): Xaa can be any naturally occurring amino acid
  misc_feature (8)..(8): Xaa can be any naturally occurring amino acid
FRIMXVLXS

SEQ ID NO: 389 - protein, 9 aa, Artificial Sequence
  Synthetic
FRIMAVLAS

(19) United States
(12) Patent Application Publication
(10) Pub. No.: US 2022/0265755 A1
(43) Pub. Date: Aug. 25, 2022

(54) ACETYLCHOLINE MODULATION OF IMMUNE FUNCTION

(71) Applicant: BCell Solutions, Inc., Colorado Spring, CO (US)

(72) Inventors: Martha Karen Newell-Rogers, Elizabeth, CO (US); Evan Newell, Seattle, WA (US)

(73) Assignee: BCell Solutions, Inc., Colorado Springs, CO (US)

(21) Appl. No.: 17/627,359

(22) PCT Filed: Jul. 16, 2020
(86) PCT No.: PCT/US2020/042399

§ 371 Date: Jan. 14, 2022

Related U.S. Application Data
(60) Provisional application No. 63/003,128, filed on Mar. 31, 2020.
(60) Provisional application No. 62/876,513, filed on Jul. 19, 2019.

(51) Int. Cl. A61K 38/00; A61P 19/02; A61P 25/00; C07K 14/315; A61P 29/00; C07K 14/31; C07K 7/08; C07K 14/165

(52) U.S. Cl. CPC A61K 38/005; A61P 19/02; A61P 25/00; C07K 14/315; A61P 29/00; C07K 14/31; C07K 7/08; C07K 14/165

(57) ABSTRACT

The invention relates to methods for modulating the neurological and immune function through targeting of acetylcholine (ACh) and CLIP. The result is wide range of new therapeutic regimens for treating, inhibiting the development of, or otherwise dealing with, a multitude of illnesses and conditions, including psychiatric or neurological disease, autoimmune disease, transplant and cell graft rejection, chronic wounds, non-neuronal immune disorders, Alzheimer's, Parkinson's Disease, Lewy Body dementia, pediatric acute neuropsychiatric syndromes (PANS), hypertension, late stage Ebola, Hantavirus, or coronavirus-induced hyperinflammatory conditions, including infections that cause a pathological “cytokine storm”, other post-infectious syndromes that result in cytokine storms in some people, and cancer, as well as novel methods of diagnosis and of introducing a treatment regimen into a subject and improving cognition and addiction cessation methods. In some embodiments the addiction is an addiction to smoking, alcohol and/or drugs.

### BACKGROUND OF INVENTION

TLR activation or antigen receptor engagement on B cells causes an increase in the number of MHC class II molecules per cell in which the antigen binding groove is filled with a cleavage product of CD74 invariant chain, known as MHC class II invariant peptide, CLIP. It has been demonstrated that a series of peptides (referred to as CLIP inhibitors) could be exchanged with CLIP for binding in the antigen binding groove of MHC class II. When TLR activated B cells were treated with peptide, both in vitro and in vivo, a selective depletion of approximately half of the B cells was observed. Thus, by removing CLIP as a “camouflage” it was determined that CLIP serves as an inhibitor of MHC class II mediated cell death of a select group of pro-inflammatory B cells.

### SUMMARY OF INVENTION

The invention is based at least in part on the discovery that acetylcholine plays an important role in modulating immune function in disease and that compounds that are acetylcholine modulators are useful in the treatment of a number of disorders. In some embodiments the acetylcholine modulators are also selective CLIP inhibitors.

The invention in some aspects is a therapeutic peptide comprising ANSGZ₁I Z₂LA Z₃GGQY (SEQ ID NO: 379), wherein Z₁ and Z₂ are each, independently, two to five amino acids, and wherein Z₃ is one to two amino acids. In some embodiments Z₁ is two amino acids. In some embodiments Z₁ is X₁R, wherein X₁ is an amino acid selected from the group consisting of I (Isoleucine) and F (Phenylalanine) and R is Arginine. In some embodiments wherein Z₂ is two amino acids. In some embodiments Z₂ is MAX₂, wherein X₂ is an amino acid selected from the group consisting of T (Threonine) and V (Valine), M is Methionine and A is Alanine. In some embodiments Z₃ is one amino acid. In some embodiments Z₃ is an amino acid selected from the group consisting of I (Isoleucine) and S (Serine).

In some embodiments the peptide comprises ANSGIRIMATLAIGGQY (SEQ ID NO: 380). In some embodiments the peptide consists essentially of ANSGIRIMATLAIGGQY (SEQ ID NO: 380). In some embodiments the peptide comprises ANSGFRIMAVLAIGGQY (SEQ ID NO: 381). In some embodiments the peptide consists essentially of ANSGFRIMAVLAIGGQY (SEQ ID NO: 381). In some embodiments the peptide comprises ANSGIRIMAVLASGGQY (SEQ ID NO: 382). In some embodiments the peptide consists essentially of ANSGIRIMAVLASGGQY (SEQ ID NO: 382).

In some embodiments Z₁ is five amino acids. In some embodiments Z₁ is LENLV (SEQ ID NO: 385), L is Leucine, E is Glutamate, N is Asparagine and V is Valine. In some embodiments Z₂ is five amino acids. In some embodiments Z₂ is LNAAS (SEQ ID NO: 386), wherein L is Leucine, N is Asparagine, A is Alanine and S is Serine. In some embodiments Z₃ is two amino acids. In some embodiments Z₃ is GT, wherein G is Glycine and T is Threonine.

In some embodiments the peptide comprises ANSGLENLVILNAASLAGTGGQY (SEQ ID NO: 387). In some embodiments the peptide consists essentially of ANSGLENLVILNAASLAGTGGQY (SEQ ID NO: 387).

In some aspects the invention is a method for treating a psychiatric or neurological disease, by administering to a subject having a psychiatric disease an isolated therapeutic compound, optionally an isolated therapeutic peptide, wherein the isolated therapeutic compound is an alpha 7 nicotinic acetylcholine (α7nACh) receptor agonist and a CLIP inhibitor, in an effective amount to treat the psychiatric or neurological disease.

In some embodiments the psychiatric or neurological disease is selected from the group consisting of: schizophrenia, mania, depression, and anxiety. In some embodiments the psychiatric or neurological disease is selected from the group consisting of attention deficit hyperactivity disorder (ADHD), Alzheimer's Disease (AD), Parkinson's Disease, Huntington's chorea, epilepsy, convulsions, Tourette syndrome, obsessive compulsive disorder (OCD), memory deficits and dysfunction, a learning deficit, a panic disorder, narcolepsy, nociception, autism, tardive dyskinesia, social phobia, pseudo dementia neuropathic pain, postoperative pain, inflammatory pain, and phantom limb pain. In some embodiments the psychiatric or neurological disease is a neurodegenerative disorder selected from the group consisting of: senile dementia and an intellectual impairment disorder.

In some aspects a method for promoting wound healing of a chronic wound, by administering to a subject having a chronic wound an isolated therapeutic compound, optionally an isolated therapeutic peptide, wherein the isolated therapeutic compound is an alpha 7 nicotinic acetylcholine (α7nACh) receptor agonist and a CLIP inhibitor, in an effective amount to treat the chronic wound is provided.

In some aspects a method for treating or improving cognition or cessation of an addiction in a subject in need thereof, by administering to the subject an isolated therapeutic compound, optionally an isolated therapeutic peptide, wherein the isolated therapeutic compound an alpha 7 nicotinic acetylcholine (α7nACh) receptor agonist and a CLIP inhibitor, in an effective amount to improve cognition or cessation of addiction relative to an untreated subject is provided. In some embodiments the addiction is an addiction to smoking, alcohol and/or drugs.

In some aspects a method for treating proliferative and non-neuronal immune disorders, by administering to a subject having a proliferative and non-neuronal immune disorders an isolated therapeutic compound, optionally an isolated therapeutic peptide, wherein the isolated therapeutic compound is an alpha 7 nicotinic acetylcholine (α7nACh) receptor agonist and a CLIP inhibitor, in an effective amount to treat the proliferative and non-neuronal immune disorders, wherein the proliferative and non-neuronal immune disorders are selected from the group consisting of autoimmune disease, Inflammatory Bowel Disease, Crohn's disease, asthma, macular degeneration (e.g., dry AMD, wet AMD), retinopathy (e.g., diabetic retinopathy), hypertension, kidney disease, preeclampsia, type 1 diabetes, arthritis (e.g., osteoarthritis, rheumatoid arthritis (RA), psoriatic arthritis and sepsis is provided.

In some embodiments the isolated therapeutic peptide comprises ANSGZ₁I Z₂LA Z₃GGQY (SEQ ID NO: 379), wherein Z₁ and Z₂ are each, independently, two to five amino acids, and wherein Z₃ is one to two amino acids. In some embodiments the isolated therapeutic compound is an isolated therapeutic peptide which is a peptide as described herein. In some embodiments the isolated therapeutic peptide comprises an isolated peptide comprising X₁RX₂X₃X₄X₅LX₆X₇ (SEQ ID NO: 383), wherein each X is an amino acid, wherein R is Arginine, L is Leucine and wherein at least one of X₂ and X₃ is Methionine. In some embodiments X₁ is Phenylalanine, wherein X₂ is Isoleucine; wherein X₃ is Methionine, wherein X₄ is Alanine, wherein X₅ is Valine, wherein X₆ is Alanine, and wherein X₇ is Serine. In some embodiments the peptide comprises FRIMX₄VLX₆S (SEQ ID NO: 388), wherein X₄ and X₆ are any amino acid. In some embodiments the peptide comprises FRIMAVLAS (SEQ ID NO: 389). In some embodiments the peptide has 9-20 amino acids. In some embodiments the peptide is non-cyclic. In some embodiments a TLR agonist is administered to the subject. In some embodiments the TLR agonist is a TLR 1, 2, 3, 4, 5, 6, 7, 8, 9, 10, and 11 agonist. In some embodiments the TLR agonist is a TLR9 agonist. In some embodiments the method further comprises administering a small molecule α7nACh Receptor agonist to the subject.

In some aspects a method for reducing adverse effects of an immune therapy in a subject being treated with the immune therapy by systemically administering to a subject receiving an immune therapy a selective α7 nicotinic acetylcholine receptor (a7 nAChR) agonist in an effective amount to reduce or eliminate a cytokine storm caused by the immune therapy, wherein the α7 nAChR agonist is a therapeutic peptide comprising ANSGZ₁I Z₂LA Z₃GGQY (SEQ ID NO: 379), wherein Z₁ and Z₂ are each, independently, two to five amino acids, and wherein Z₃ is one to two amino acids, wherein the immune therapy is a checkpoint inhibitor therapy or cell therapy such as CAR-T cell therapy is provided.

In some aspects a method for reducing adverse effects of an immune therapy in a subject being treated with the immune therapy by systemically administering to a subject receiving an immune therapy an isolated therapeutic compound, optionally an isolated therapeutic peptide and a small molecule nicotinic acetylcholine (α7nACh) Receptor agonist is provided. In some embodiments the isolated therapeutic compound is an α7nACh receptor agonist and a CLIP inhibitor in an effective amount to reduce or eliminate a cytokine storm caused by the immune therapy. In some embodiments the immune therapy is a checkpoint inhibitor therapy or cell therapy such as CAR-T cell therapy. In some embodiments the subject has cancer.

In some aspects a method for reducing adverse effects of an immune response in a subject having a hyper-immune response by systemically administering to the subject an isolated therapeutic compound, optionally an isolated therapeutic peptide and a small molecule nicotinic acetylcholine (α7nACh) Receptor agonist is provided.

In some embodiments, the isolated therapeutic compound is administered to a subject having an infectious disease. In some embodiments the subject having an infectious disease is administered the isolated therapeutic agent in an effective amount to reduce or eliminate a cytokine storm caused by an infectious agent causing the disease. In some embodiments, the infectious agent is Ebola, SARS, SARS-CoV-2, or MERS. In other embodiments the infectious disease is caused by an infectious agent selected from the group consisting of Streptococcus, Staphylococcus, Coronaviruses, and Hantaviruses.

In some embodiments, the subject having a hyper-immune response has a post-infectious chronic inflammatory syndrome.

In some aspects the invention is a method for treating a subject having a post-infectious chronic inflammatory syndrome, by identifying a subject having a post-infectious chronic inflammatory syndrome and systemically administering to the subject an isolated therapeutic peptide in an effective amount to treat the a post-infectious chronic inflammatory syndrome.

In other aspects the invention is a method of treating a subject having a disorder associated with blood brain barrier (BBB) permeability by identifying a subject having a disorder associated with BBB permeability, and administering to the subject an isolated selective α7 nicotinic acetylcholine receptor (α7 nAChR) agonist in an effective amount to treat the disorder associated with BBB, wherein the selective α7 nAChR agonist is an isolated therapeutic peptide. In some embodiments the isolated therapeutic peptide comprises ANSGZ₁I Z₂LA Z₃GGQY (SEQ ID NO: 379), wherein Z₁ and Z₂ are each, independently, two to five amino acids, and wherein Z₃ is one to two amino acids. In other embodiments the isolated therapeutic peptide comprises an isolated peptide comprising X₁RX₂X₃X₄X₅LX₆X₇ (SEQ ID NO: 383), wherein each X is an amino acid, wherein R is Arginine, L is Leucine and wherein at least one of X₂ and X₃ is Methionine. In other embodiments a small molecule α7nACh Receptor agonist is administered to the subject.

In some aspects a method of treating a subject having cancer by systemically administering to the subject an isolated therapeutic compound, optionally an isolated therapeutic peptide and a small molecule nicotinic acetylcholine (α7nACh) receptor agonist, wherein the isolated therapeutic compound is a α7nACh receptor agonist and a CLIP inhibitor in an effective amount to treat the cancer is provided.

In other aspects, a method of treating a subject having cancer, by systemically administering to the subject an isolated selective α7 nicotinic acetylcholine receptor (α7 nAChR) agonist in an effective amount to treat the cancer, wherein the selective α7 nAChR agonist is a therapeutic peptide comprising ANSGZ₁I Z₂LA Z₃GGQY (SEQ ID NO: 379), wherein Z₁ and Z₂ are each, independently, two to five amino acids, and wherein Z₃ is one to two amino acids is provided. In some embodiments the subject has a melanoma.

In some embodiments the method further comprises administering to the subject a checkpoint inhibitor. In some embodiments the checkpoint inhibitor is an antibody selected from an anti-CTLA4 antibody or antigen-binding fragment thereof that specifically binds CTLA4, an anti-PD1 antibody or antigen-binding fragment thereof that specifically binds PD1, an anti-PD-L1 antibody or antigen-binding fragment thereof that specifically binds PD-L1, an antibody selected from an anti-VISTA antibody or antigen-binding fragment thereof that specifically binds VISTA and a combination thereof. In some embodiments the checkpoint inhibitor is an anti-PD-L1 antibody selected from atezolizumab, avelumab, or durvalumab. In some embodiments the checkpoint inhibitor is an anti-CTLA-4 antibody selected from tremelimumab or ipilimumab. In some embodiments the checkpoint inhibitor is an anti-PD1 antibody selected from nivolumab or pembrolizumab.

A method of treating a subject having Alzheimer's disease by administering to the subject an isolated therapeutic compound, optionally an isolated therapeutic peptide and a small molecule nicotinic acetylcholine (α7nACh) receptor agonist, wherein the isolated therapeutic compound is an α7nACh receptor agonist and a CLIP inhibitor in an effective amount to treat the Alzheimer's disease is provided in some aspects of the invention.

In some aspects a method of treating a subject having Alzheimer's disease by administering to the subject an isolated selective α7 nicotinic acetylcholine receptor (α7 nAChR) agonist in an effective amount to treat the Alzheimer's disease, wherein the selective α7 nAChR agonist is a therapeutic peptide comprising ANSGZ₁I Z₂LA Z₃GGQY (SEQ ID NO: 379), wherein Z₁ and Z₂ are each, independently, two to five amino acids, and wherein Z₃ is one to two amino acids is provided.

A method of treating a subject having multiple sclerosis by administering to the subject an isolated therapeutic compound, optionally an isolated therapeutic peptide and a small molecule nicotinic acetylcholine (α7nACh) receptor agonist, wherein the isolated therapeutic compound is a α7nACh receptor agonist and a CLIP inhibitor in an effective amount to treat the multiple sclerosis is provided in some aspects.

In some aspects a method of treating a subject having multiple sclerosis by administering to the subject an isolated selective α7 nicotinic acetylcholine receptor (α7 nAChR) agonist in an effective amount to treat the multiple sclerosis, wherein the selective α7 nAChR agonist is a therapeutic peptide comprising ANSGZ₁I Z₂LA Z₃GGQY (SEQ ID NO: 379), wherein Z₁ and Z₂ are each, independently, two to five amino acids, and wherein Z₃ is one to two amino acids is provided.

In some embodiments the isolated therapeutic peptide comprises ANSGZ₁I Z₂LA Z₃GGQY (SEQ ID NO: 379), wherein Z₁ and Z₂ are each, independently, two to five amino acids, and wherein Z₃ is one to two amino acids. In some embodiments the isolated therapeutic compound is an isolated therapeutic peptide which is a peptide as disclosed herein. In some embodiments the isolated therapeutic peptide comprises an isolated peptide comprising X₁RX₂X₃X₄X₅LX₆X₇ (SEQ ID NO: 383), wherein each X is an amino acid, wherein R is Arginine, L is Leucine and wherein at least one of X₂ and X₃ is Methionine. In some embodiments X₁ is Phenylalanine, wherein X₂ is Isoleucine; wherein X₃ is Methionine, wherein X₄ is Alanine, wherein X₅ is Valine, wherein X₆ is Alanine, and wherein X₇ is Serine. In some embodiments the peptide comprises FRIMX₄VLX₆S (SEQ ID NO: 388), wherein X₄ and X₆ are any amino acid. In some embodiments the peptide comprises FRIMAVLAS (SEQ ID NO: 389). In some embodiments the peptide has 9-20 amino acids. In some embodiments the peptide is non-cyclic. In some embodiments a TLR agonist is administered to the subject. In some embodiments the TLR agonist is a TLR 1, 2, 3, 4, 5, 6, 7, 8, 9, 10, 11 agonists. In some embodiments the TLR agonist is a TLR9 agonist. In some embodiments the method further comprises administering a small molecule α7nACh Receptor agonist to the subject.

A composition of a therapeutic peptide and a small molecule nicotinic acetylcholine (α7nACh) receptor agonist, wherein the isolated therapeutic compound a α7nACh receptor agonist and a CLIP inhibitor is provided in some aspects.

In some aspects a kit having a container housing a therapeutic peptide, wherein the therapeutic peptide is a nicotinic acetylcholine (α7nACh) receptor agonist and a CLIP inhibitor, a container housing a small molecule α7nACh receptor agonist, and instructions for administering the combination of the therapeutic peptide and the small molecule to a subject in need thereof is provided.

In other aspects a method of administering to a subject receiving an organ transplant from a donor an isolated therapeutic compound, optionally an isolated therapeutic peptide, wherein the isolated therapeutic compound a donor specific CLIP inhibitor in an effective amount to suppress an immune response in the subject to the donor organ is provided. In some embodiments the method further comprises administering a small molecule nicotinic acetylcholine (α7nACh) receptor agonist to the subject.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1C. Activation of TLR 9 results in increased alpha 7 nicotinic acetylcholine receptor expression on B cells as shown in FIGS. 1A-1C. 1A shows that 9% of the total spleen cells are B cells that express the alpha 7 nAChR. 1B shows that a total of 37.6% of the total splenocytes express alpha 7 nAChR after CpG treatment. 1C shows the relative level of expression of α7 nAChR per cell increases as a result of CpG treatment.

FIGS. 2A-2E. The effects of Competitive Antagonist Peptide can be reversed by Methyllylaconitine (MLA), a specific inhibitor of alpha 7 nAChR activation as shown in FIGS. 2A-2E. Animals were randomized into groups of vehicle treated (2A), Complete Freund's Adjuvant (CFA) treated 2B), CFA treated and Peptide treated (2C), or CFA treated, Peptide treated, and treated with MLA (2D). FIG. 2E shows the Peptide induced reduction in CLIP+ B cells is reversed by treatment with MLA.

FIGS. 3A-3D. The figures show that CpG-mediated CLIP expression protects B cells from MHCII mediated cell death. FIG. 3A shows the percent change in cell death of resting, C57B/6 B cells treated with anti-MHCII (M5/114) or the isotype control rat IgG2b. FIG. 3B shows that % change in cell death of CpG activated C57Bl6 B cells treated with peptide (TPP), anti-MHCII (M5114), or peptide followed by anti-MHCII (M5114). FIG. 3C shows the % change in cell death of CpG activated Invariant Chain deficient (Ii) C57Bl6 B cells treated with peptide (TPP), anti-MHCII (M5114), or peptide followed by anti-MHCII (M5114). FIG. 3D shows the mean fluorescence intensity (MFI) of MHCII on B cell activated as labeled for 48 hours. C57B/6 (solid black bars), IiDef (solid grey bars). * designates a p value <0.05 compared to the selected group.

FIGS. 4A-4F Still frame images from intravital microscopy video recorded 24 hours post-treatments and following injection of FITC-dextran (3 kDa, green): 4A, 4B, and 4C. extravasation from, or rhodamine 6G stained (red) 4D, 4E, and 4F, lymphocytes adhering or rolling (yellow arrows) within pial venules of mice, in vivo: No treatment (4A) and (4D); Intranasal Streptococcus Pyogenes (4B) and (4E); and Intranasal Streptococcus Pyogenes with Intraperitonal Administration of Peptide Therapy (4C) and (4F).

FIGS. 5A-5D Effect of intranasal Streptococcus pyogenes infection on BBB permeability and lymphocyte behavior. 5A) Extravasation of FITC-dextran dye (3 kDa) from pial venules increased 24 hours post-infection. Intensity normalized and compared to intranasal saline (control) treated animals: *, p<0.05 (Students t-test, one-tail). (5B) Quantification of number of adherent lymphocytes observed in a 100 um length of pial venule during 1 minute. Intranasal infection increased number of adherent cells 24 hours post-infection. TPP treatment significantly reversed infection-induced adherence. Adherent cells did not move from original location throughout the course of observation (1 minute): **, p<0.01; ***, p<0.001 (One-way ANOVA). (5C) Quantification of number of rolling lymphocytes observed in a 100 um length of pial venule during 1 minute. Intranasal infection increased number of rolling cells 24 hours post-infection. TPP treatment reduced infection-induced rolling. Rolling cells moved slower than freely floating cells (within center of venule), but never fully-adhered to venule wall throughout the course of observation (1 minute): **, p<0.01 (One-way ANOVA). (5D) Qualitative assessment of the size (um) of adherent cells measured in (5B). Infection, even with TPP treatment, markedly increased the size of observed, adherent lymphocytes.

FIGS. 6A-6D Effect of Pam-3-Cys (P3C, a TLR 1/2 agonist) on BBB permeability and lymphocyte behavior. 6A) Extravasation of FITC-dextran dye (3 kDa) from pial venules increased 24 hours post-i.p. P3C. Intensity normalized and compared to intranasal saline (control) treated animals: *, p<0.05 (Students t-test, one-tail). (6B) Quantification of number of adherent lymphocytes observed in a 100 um length of pial venule during 1 minute. P3C increased number of adherent cells 24 hours post-infection. TPP treatment significantly reversed P3C-induced adherence. Adherent cells did not move from original location throughout the course of observation (1 minute): **, p<0.01; ***, p<0.001 (One-way ANOVA). (6C) Quantification of number of rolling lymphocytes observed in a 100 um length of pial venule during 1 minute. P3C increased number of rolling cells 24 hours post-infection. TPP treatment P3C-induced rolling. Rolling cells moved slower than freely floating cells (within center of venule), but never fully-adhered to venule wall throughout the course of observation (1 minute): **, p<0.01 (One-way ANOVA). (6D) Qualitative assessment of the size (um) of adherent cells measured in (6B). P3C, even with TPP treatment, markedly increased the size of observed, adherent lymphocytes.

FIG. 7. Social interaction after TBI model. The data are relative to baseline (BL), and reveal significantly decreased social interaction by TBI mice at 9, 19 and 58 days post-TBI.

FIG. 8. Collagen-induced arthritis model. Animals were injected with a collagen emulsion in Complete Freunds Adjuvant (CFA) and treated with either CAP during early disease development or once arthritic disease was established. Results demonstrate a dose dependent reduction in cytokines, particularly in the established (late stage) disease model.

FIG. 9. CAP reduces IL-17 production in a model of preeclampsia. Pregnant mice were treated with the TLR3 or TLR8 agonists to induce symptoms of preeclampsia, followed by CAP treatment. CAP significantly reduced IL-17a.

FIG. 10. TBI-induced changes in brain cytokines. TBI induced increase in brain IL1b, IL6 and IL17 are reversed by CAP. Cap was administered i.p. 30 minutes after TBI and brains were harvested 24 hours after TBI.

FIGS. 11A-11B. CAP depletion of B cells in the CFA-induced arthritis model. In this study, CAP was administered at 3 days after CFA, then once daily every 3 days, until day 10 after CFA. CAP selectively depletes pro-inflammatory B cell populations.

FIG. 12. CAP depletion of pro-inflammatory CLIP+ B cells is reversed by antagonizing α7 nAChR, CAP-mediated B cell depletion is reversed by MLA, a specific inhibitor of To determine if CAP mediated B cell depletion involves activation of α7 nAChR, mice were injected with CFA followed 48 hours later with CAP treatment without or with treatment with MLA, a specific inhibitor of α7nAChR. The results demonstrate that CAP-dependent B cell depletion was reversed by treatment with MLA.

FIG. 13. CAP treatment induces expansion of CD8+ T cells.

FIG. 14. CAP treatment resulted in CD8 T cell expansion that was reversed by treatment with methyllycaconitine (MLA), a specific inhibitor of α7nAChR.

FIG. 15. CAP reverses inflammatory characteristics in a preeclampsia model. Pregnant mice were treated with TLR agonists (R837, Clo97, and PIC) to induce symptoms of preeclampsia followed by CAP treatment (shown as TPP). CAP significantly reduced blood pressure.

FIGS. 16A-16B. Treatment with CAP restores afferent arteriolar autoregulatory behavior in chronic LPS (0.1 mg/kg/day) treated kidneys. Each data point represents the mean±SE. * P<0.05 vs. control diameter in the same group. ‡ P<0.05 vs. control for the same perfusion pressure. # P<0.05 vs. CAP for the same perfusion pressure.

FIG. 17. CAP reduces peripheral pro-inflammatory cells. TBI is traumatic brain injury. FPI is fluid percussion injury.

### DETAILED DESCRIPTION

The present invention provides new insights into the role of acetylcholine (ACh) in disease and presents novel approaches to modulating the immune function through manipulation and/or engagement of the alpha 7 nicotinic acetylcholine receptor alone or in combination with targeting of invariant chain/CD74 and CLIP. The result is a wide range of new therapeutic regimens for treating or inhibiting the development or progression of a multitude of illnesses and conditions, including psychiatric or neurological disease, autoimmune disease, transplant and cell graft rejection, chronic wounds, non-neuronal immune disorders, Alzheimer's, Parkinson's Disease, Lewy Body dementia, pediatric acute neuropsychiatric syndromes (PANS), hypertension, late stage Ebola, Hantavirus, or coronavirus-induced hyperinflammatory conditions, including infections that cause a pathological “cytokine storm”, other post-infectious syndromes that result in cytokine storms in some people, and cancer, as well as novel methods of diagnosis and of introducing a treatment regimen into a subject and improving cognition and addiction cessation methods. In some embodiments the addiction is an addiction to smoking, alcohol and/or drugs.

The presence of CLIP on a B cell surface can be undesirable. If CLIP gets removed from the groove by a self-antigen, the B cell would be in a position to present self-antigens to self-reactive T cells, a process that could lead to auto reactivity and autoimmune disease. For some B cells this may result in death to the B cell by a nearby killer cell, such as a natural killer (NK) cell or a CD4 T cell, unless the antigen receptor on the B cell has engaged antigen. Antigen recognition provides a survival signal for the B cell. If the autoreactive B cell is not removed and it encounters a CD4+ T cell that can recognize that antigen the B cell and T cell may work together to lead to T cell activation and in some cases deletion of the B cell or antigen presenting cell (ref: Setterblad, N., et al. Cognate MHC-TCR interaction leads to apoptosis of antigen-presenting cells. J Leukoc Biol 75, 1036-1044 (2004). Newell et al discovered that synthetic peptides could be used to treat disease by altering the binding of CLIP and antigen to MHC on B cells, and thus altering B cell activity.

It has now been discovered that synthetic peptides, some with overlapping structures with peptides derived from other proteins, including beta amyloid peptides or peptides from myelin basic protein, manipulate acetylcholine receptor activity and thus may be used to treat a variety of diseases. It has been demonstrated herein that the synthetic peptides have a high probability of binding to the alpha 7 nicotinic acetylcholine receptor (α7nAChR) and functioning as α7nAChR agonists. Through in vitro and in vivo data disclosed in the Examples, it has been demonstrated that the biologic effects of synthetic peptides following TLR activation were reversed with a specific inhibitor of α7 nAChR called methyllycaconitine (MLA). It was also discovered that TLR activation causes the cell surface expression of α7nAChR receptors on B cells. Peptides with these particular features, both via their primary sequences, secondary, and tertiary structures, have the unique qualities of both competitively binding in the groove of certain MHC class II alleles (i.e., functioning as expanding TLR activated B cells.

Previous work had shown that when conventional CD4 T cells are activated with anti-CD3 and anti-CD28 (as surrogates for antigen receptor engagement and co-stimulation), these T cells were induced to express message for choline acetyl transferase (CHAT) and potentially nicotinic or muscarinic AChR. The synthetic peptides disclosed herein bind to the alpha 7 nAChR on the APC, such as a B cell, and it is believed that these APCs in turn activate 2 antigen specific Tregs (regulatory T cells) that can control an immune response to “self” antigens. When conventional CD4 T cells recognize tumor antigens, resulting in cell surface expression of some AChR on the surface of the anti-tumoral T cell, the tumor cells respond (potentially via PD1:PDL1 or CTLA-4:B7 interactions) by releasing acetylcholine that converts the anti-tumoral CD4 T cell into a Treg specifically to downregulate the anti-tumoral antigen response.

Based on the findings of the invention, the identification of novel targets for immunotherapy by disrupting the pathways in either the tumor, that is producing ACh or the T cells, that are responding to ACh, provide a novel method for potentiating a specific anti-tumoral immune response, as well as the treatment of a variety of other diseases. Use of this technology could block the conversion of anti-tumoral T cells to Tregs in an antigen specific manner, avoiding potentially adverse effects of immune check-point inhibitor and cellular therapies that can result in lethal autoreactivity. Thus, the compositions may be used together with immune provoking anti-cancer therapies.

Additionally, ACh receptor expression, production of ACh, and the conversion of CD4 T cells or CD8 T cells to CD4 or CD8 Treg cells could be manipulated using the peptides described herein in order to regulate self-non-self-discrimination and maintenance of self-tolerance. Thus, novel targets to control autoimmune diseases and chronic inflammation and a novel approach to prevent graft rejection and graft versus host disease are also provided according to aspects of the invention. These discoveries have important implications in the treatment of disease.

Inflammation, infection, autoimmunity, and brain injuries increase blood brain barrier (BBB) permeability, allowing inflammatory cytokines, chemokines, other blood-born proteins, and immune cells (peripheral macrophages, B and T cells, including CD4+ T cells) to enter the brain parenchyma and promote disease progression. BBB permeability is a well-recognized characteristic of a number of neurological diseases. Recent work suggests that pericytes are a key cellular regulator of the BBB, and in response to alpha-synuclein binding to the pericyte, inflammatory cytokines are released that can cause BBB permeability and influx of inflammatory molecules and immune cells into the brain.

The methods disclosed herein also involve targeting these pro-inflammatory cells to cause their elimination. The therapeutic peptide immunotherapy disclosed herein creates an interaction between the nervous system and the immune system that contributes to reduction in inflammation. This reduction is not only by the elimination of unwanted pro-inflammatory cells, but also by triggering the expansion of cell populations that are anti-inflammatory. As discussed above, it has been discovered that the therapeutic peptide therapy activates the newly discovered cholinergic anti-inflammatory pathway, specifically via the α7 nicotinic acetylcholine receptor (α7nAChR). The α 7nAChR receptors are widely expressed in the central nervous system, peripheral tissues, and cells of the immune system. After activation, a 7nAChR engagement appears to activate the cholinergic anti-inflammatory cascade. While this pathway was first discovered in the periphery, its newly established anti-inflammatory role in the nervous system highlights the importance of this anti-inflammatory pathway as a therapeutic target, and emphasizes the importance of the therapeutic peptides disclosed herein. These new discoveries are significant for the treatment of chronic inflammatory, cognitive, and neurodegenerative diseases, such as Parkinson's disease.

Activation of the α7 nicotinic acetylcholine receptor upregulates BBB function by increasing the activity of the tight junction proteins known as claudin-5 and occluding expression in brain endothelium. As demonstrated herein in a mouse model of intranasal streptococcal infection, the therapeutic peptides were able to reverse the BBB permeability associated with both infection and TLR activation. These data highlight the important role of the therapeutic peptides disclosed herein in treating diseases that are caused by, or accelerated by, BBB permeability.

In an adaptive immune response, recognition of antigens associated with Major Histocompatibility Complex (MHC) encoded molecules expressed on the cell surface of an antigen presenting cell (APC) is the first step required for T cell activation. This important requirement both preserves the antigen specificity of the T cell and confers MHC restriction, which limits the T cell's response to those antigens associated with one's own MHC. CD4+ T cell activation requires recognition of antigens associated with MHC class II along with co-stimulation, which can be either stimulatory or inhibitory (such as the inhibition conferred by the receptor-ligand pairs PD1:PDL1 or CD86:CTLA4). Thus, the nature of CD4+ T cell activation is determined by the balance of signals received by the T cell and by the APC. In parallel, T cell recognition of antigen can exert direct effects on the cell being recognized, including both professional APCs, such as macrophages, dendritic cells, and B cells, or non-professional APCs such as endothelial cells. Specifically, engagement of MHC class II by the T cell receptor (TCR) can deliver signals to the MHC class II-bearing cell and, under the appropriate circumstances, MHC class II engagement can result in cell death of the MHC class II-expressing cell. This step in the adaptive immune response can also result in deletion of unwanted and pro-inflammatory cells.

Signaling via Toll Like Receptors, such as TLR 1 and 2 that are involved in synuclein-mediated neuroinflammation, activating the MyD88 pathway and resulting in the expansion of pro-inflammatory cells that express MHC class II molecules in which the peptide binding groove is filled with the MHC class II associated invariant peptide (CLIP). Under these circumstances, CLIP appears to protect the expanding pro-inflammatory cells from being recognized and removed.

To address the problem of CLIP expression in MHC class II as a cell survival mechanism for pro-inflammatory cells, the therapeutic peptides disclosed herein were designed to have a binding coefficient for MHC class II that is greater than that of CLIP in known human MHC class II alleles. This leads to a preferential binding of these peptides to MHC class II on cell surface CLIP+ cells. With the therapeutic peptide displayed in MHC class II, the cell can be recognized by a CD4+ T cell as presenting a “foreign/unwanted” antigen, which then focuses a CD4+ T cell response on the peptide expressing cell. Thus, CD4+ T cell response is narrowly focused to unwanted cells which have been converted to peptide-expressing cells. The peptides have been validated for this capacity to deplete inflammatory cells in multiple animal models, including multiple mouse models of chronic inflammatory disease, including chronic kidney disease and preeclampsia.

The discoveries described herein demonstrate that the therapeutic peptide immunotherapy creates an interaction between the nervous system and the immune system that contributes to the reduction in inflammation, not only by the elimination of unwanted pro-inflammatory cells, but also by the expansion of cell populations that are anti-inflammatory. The peptides have been used in a mouse model of brain trauma injury in which the peptide prevents or stops further progression of neurodegeneration.

The compounds useful in the methods of the invention are α7nAChR agonists. An α7nAChR agonist as used herein refers to a compound, preferably a peptide, that binds to the α7nAChR and activates the α7nAChR stimulation cascade.

In some embodiments the α7nAChR agonists are also CLIP inhibitors. A CLIP inhibitor as used herein is any molecule that reduces the association of a CLIP molecule with MHC by binding to the MHC and blocking the CLIP-MHC interaction. The CLIP inhibitor may function by displacing CLIP from the surface of a CLIP molecule expressing cell. A CLIP molecule expressing cell is a cell that has MHC class I or II on the surface and includes a CLIP molecule within that MHC. Such cells include B cells, neurons, oligodendrocytes, microglial cells, astrocytes, heart cells, pancreatic beta cells, intestinal epithelial cells, lung cells, epithelial cells lining the uterine wall, and skin cells.

The CLIP molecule, as used herein, refers to intact CD74 (also referred to as invariant chain), as well as the naturally occurring proteolytic fragments thereof. CLIP is one of the naturally occurring proteolytic fragments thereof. The function of the CLIP molecule in this invention is mainly as an MHC class II chaperone. MHC class II molecules are heterodimeric complexes that present foreign antigenic peptides on the cell surface of antigen-presenting cells (APCs) to CD4⁺ T cells. MHC class II synthesis and assembly begins in the endoplasmic reticulum (ER) with the non-covalent association of the MHC α and β chains with trimers of CD74. CD74 is a non-polymorphic type II integral membrane protein; murine CD74 has a short (30 amino acid) N-terminal cytoplasmic tail, followed by a single 24 amino acid transmembrane region and an ˜150 amino acid long lumenal domain. Three MHC class II αβ dimers bind sequentially to a trimer of the CD74 to form a nonameric complex (αβIi)3, which then exits the ER. After being transported to the trans-Golgi, the αβIi complex is diverted from the secretory pathway to the endocytic system and ultimately to acidic endosome or lysosome-like structures called MHC class I or II compartments.

The N-terminal cytoplasmic tail of CD74 contains two extensively characterized dileucine-based endosomal targeting motifs. These motifs mediate internalization from the plasma membrane and from the trans-Golgi network. In the endocytic compartments, the CD74 chain is gradually proteolytically processed, leaving only a small fragment, the class II-associated CD74 chain peptide (CLIP), bound to the released αβ dimers. The final step for MHC class II expression requires interaction of αβ-CLIP complexes with another class II-related αβ dimer, called HLA-DM in the human system. This drives out the residual CLIP, rendering the αβ dimers ultimately competent to bind antigenic peptides, which are mainly derived from internalized antigens and are also delivered to the endocytic pathway. The peptide-loaded class II molecules then leave this compartment by an unknown route to be expressed on the cell surface and surveyed by CD4⁺ T cells.

The compounds that are both α7nAChR agonists and CLIP inhibitors include peptides and small molecules that can both bind to an α7nAChR and replace CLIP in a cell surface MHC. The compounds having dual selectivity may be selected based on their ability to bind to both α7nAChR and MHC, equivalently, or in some embodiments to have a stronger binding affinity to α7nAChR. Not all α7nAChR agonists are CLIP inhibitors and not all CLIP inhibitors are α7nAChR agonists.

In some embodiments the therapeutic compound that is an α7nAChR agonist or an α7nAChR agonist and CLIP inhibitor is a peptide. A number of peptides useful as α7nAChR agonists and optionally for displacing CLIP molecules are described herein. For instance a number of peptide sequences that function in this manner are disclosed in Table 1.

TABLE 1

| Peptide sequence | Score | SEQ ID NO |
|---|---|---|
| CARQEDTAMVYYFDYW | 1.055417727 | 1 |
| NLFLFLFAV | 1.026491736 | 2 |
| NLFLFLFAV | 1.026491736 | 3 |
| FLWSVFMLI | 1.02084488 | 4 |
| FLWSVFMLI | 1.02084488 | 5 |
| FLWYSTSESH | 1.015308924 | 6 |
| KITLFVIVPV | 1.007232472 | 7 |
| TLFVIVPVL | 1.007232472 | 8 |
| KITLFVIVPV | 1.007232472 | 9 |
| TLFVIVPVL | 1.007232472 | 10 |
| FLQYSTSECH | 0.996853489 | 11 |
| FLIVLSVAL | 0.995295062 | 12 |
| FLIVLSVAL | 0.995295062 | 13 |
| KLQVFLIVL | 0.99521825 | 14 |
| KLQVFLIVL | 0.99521825 | 15 |
| VMNILLQYVV | 0.992763193 | 16 |
| VMNILLQYVV | 0.992763193 | 17 |
| KYCLITIFL | 0.991237267 | 18 |
| RMMEYGTTMV | 0.986139699 | 19 |
| RMMEYGTTMV | 0.986139699 | 20 |
| VIVMLTPLV | 0.985136317 | 21 |
| VIVMLTPLV | 0.985136317 | 22 |
| KYNIANVFL | 0.984307007 | 23 |
| ALYLVCGER | 0.97797231 | 24 |
| LYLVCGERGF | 0.97797231 | 25 |
| LYLVCGERG | 0.97797231 | 26 |
| LYLVCGERI | 0.97797231 | 27 |
| ALYLVCGER | 0.97797231 | 28 |
| LYLVCGERGF | 0.97797231 | 29 |
| SHLVEALYLVCGERG | 0.97797231 | 30 |
| FLFAVGFYL | 0.96802159 | 31 |
| FLFAVGFYL | 0.96802159 | 32 |
| KYQAVTTTL | 0.963071576 | 33 |
| GIVEQCCTSI | 0.948238574 | 34 |
| IVEQCCTSI | 0.948238574 | 35 |
| GIVEQCCTSI | 0.948238574 | 36 |
| IVEQCCTSI | 0.948238574 | 37 |
| RLLCALTSL | 0.899728815 | 38 |
| RLLCALTSL | 0.899728815 | 39 |
| SHLVEELYLVAGEEG | 0.878587897 | 40 |
| VLFGLGFAI | 0.868387328 | 41 |
| VLFGLGFAI | 0.868387328 | 42 |
| VLFGLGFAI | 0.868387328 | 43 |
| IILVEALYLV | 0.860198614 | 44 |
| FLYGALLLA | 0.783025002 | 45 |
| FLYGALLLA | 0.783025002 | 46 |
| LNIDLLWSV | 0.763415969 | 47 |
| LNIDLLWSV | 0.763415969 | 48 |
| LNIDLLWSV | 0.763415969 | 49 |
| FQDENYLYL | 0.698830498 | 50 |
| HLVEALYLV | 0.621310823 | 51 |
| HLVEALYLV | 0.621310823 | 52 |
| SLYQLENYC | 0.607675548 | 53 |
| SLYQLENYC | 0.607675548 | 54 |
| HLCGSHLVEA | 0.607650742 | 55 |
| HLCGSHLVEA | 0.607650742 | 56 |
| FLEYSTSECH | 0.586899948 | 57 |
| RLAGQFLEEL | 0.561081499 | 58 |
| RLAGQFLEEL | 0.561081499 | 59 |
| VLFSSDFRI | 0.556444788 | 60 |
| VLFSSDFRI | 0.556444788 | 61 |
| SLAAGVKLL | 0.506937884 | 62 |
| SLAAGVKLL | 0.506937884 | 63 |
| FLEYDTYEC | 0.476510754 | 64 |
| NLAQTDLATV | 0.462879735 | 65 |
| NLAQTDLATV | 0.462879735 | 66 |
| FLKYSTSECH | 0.423136765 | 67 |
| SLLLELEEV | 0.396832622 | 68 |
| SLLLELEEV | 0.396832622 | 69 |
| YLLLRVLNI | 0.236657274 | 70 |
| YLLLRVLNI | 0.236657274 | 71 |
| VYLKTNVFL | 0.153434604 | 72 |
| VYLKTNLFL | 0.153434604 | 73 |
| FLKEPMGECH | 0.118716711 | 74 |
| SLSPLQAEL | 0.093448249 | 75 |
| SLSPLQAEL | 0.093448249 | 76 |
| FLQLLRRECH | 0.076711376 | 77 |
| FLEQVKHKYY | 0.076195273 | 78 |
| ALWMRLLPL | 0.073026254 | 79 |
| LWMRLLPLL | 0.073026254 | 80 |
| ALWMRLLPL | 0.073026254 | 81 |
| LWMRLLPLL | 0.073026254 | 82 |
| SLQPLALEG | 0.071567506 | 83 |
| SLQPLALEG | 0.071567506 | 84 |
| LMWAKIGPV | 0.059650495 | 85 |
| LMWAKIGPV | 0.059650495 | 86 |
| RTFDPHFLRV | 0.051917077 | 87 |
| RTFDPHFLRV | 0.051917077 | 88 |
| KYNKANAFL | 0.037685029 | 89 |
| KYNKANVFL | 0.037685029 | 90 |
| KVEDPFYWV | 0.036664924 | 91 |
| KVEDPFYWV | 0.036664924 | 92 |
| TPKTRREAEDL | 0.028483144 | 93 |
| TPKTRREAEDL | 0.028483144 | 94 |
| FLEYRTKEC | 0.022310516 | 95 |
| ALEGSLQKR | 0.020585742 | 96 |
| ALEGSLQKR | 0.020585742 | 97 |
| FLRVPCWKI | 0.020401172 | 98 |
| FLRVPCWKI | 0.020401172 | 99 |
| FYTPKTRRE | 0.014662247 | 100 |
| FYTPKTRRE | 0.014662247 | 101 |
| SLQKRGIVEQ | 0.011873143 | 102 |
| SLQKRGIVEQ | 0.011873143 | 103 |
| ERGFFYTPK | 0.011258044 | 104 |
| ERGFFYTPK | 0.011258044 | 105 |
| LVCGERGFF | 0.009076724 | 106 |
| VCGERGFFYT | 0.009076724 | 107 |
| GERGFFYT | 0.009076724 | 108 |
| LVCGERGFF | 0.009076724 | 109 |
| VCGERGFFYT | 0.009076724 | 110 |
| GERGFFYT | 0.009076724 | 111 |
| QLARQQVHV | 0.004539939 | 112 |
| QLARQQVHV | 0.004539939 | 113 |

In some embodiments the therapeutic peptide comprises or consists essentially of a sequence selected from any of SEQ ID NOs. 1-50. In some embodiments the therapeutic peptide comprises or consists essentially of a sequence selected from any of SEQ ID NOs. 1-69. In some embodiments the therapeutic peptide comprises or consists essentially of a sequence selected from any of SEQ ID NOs. 1-71. In some embodiments the therapeutic peptide comprises or consists essentially of a sequence selected from any of SEQ ID NOs. 1-113. In some embodiments the therapeutic peptide comprises or consists essentially of a sequence selected from any of SEQ ID NOs. 1-10. In some embodiments the therapeutic peptide comprises or consists essentially of a sequence selected from any of SEQ ID NOs. 1-20. In some embodiments the therapeutic peptide comprises or consists essentially of a sequence selected from any of SEQ ID NOs. 1-30. In some embodiments the therapeutic peptide comprises or consists essentially of a sequence selected from any of SEQ ID NOs. 1-40. In some embodiments the therapeutic peptide comprises or consists essentially of a sequence selected from any of SEQ ID NOs. 50-69. In some embodiments the therapeutic peptide comprises or consists essentially of a sequence selected from any of SEQ ID NOs. 69-71. In some embodiments the therapeutic peptide comprises or consists essentially of a sequence selected from any of SEQ ID NOs. 72-113.

In some embodiments the therapeutic peptide that is an α7nAChR agonist or an α7nAChR agonist and CLIP inhibitor has the following sequence:

ANSGZ₁I Z₂LA Z₃GGQY (SEQ ID NO: 379), wherein Z₁ and Z₂ are each, independently, two to five amino acids, and wherein Z₃ is one to two amino acids. In some embodiments Z₁ is two amino acids. In some embodiments Z₁ is X₁R, wherein X₁ is an amino acid selected from the group consisting of I (Isoleucine) and F (Phenylalanine) and R is Arginine. In some embodiments wherein Z₂ is two amino acids. In some embodiments Z₂ is MAX₂, wherein X₂ is an amino acid selected from the group consisting of T (Threonine) and V (Valine), M is Methionine and A is Alanine. In some embodiments Z₃ is one amino acid. In some embodiments Z₃ is an amino acid selected from the group consisting of I (Isoleucine) and S (Serine).

In some embodiments the peptide comprises ANSGIRIMATLAIGGQY (SEQ ID NO: 380); ANSGFRIMAVLAIGGQY (SEQ ID NO; 381); or ANSGIRIMAVLASGGQY (SEQ ID NO: 382).

In some embodiments Z₁ is five amino acids. In some embodiments Z₁ is LENLV (SEQ ID NO: 385), L is Leucine, E is Glutamate, N is Asparagine and V is Valine. In some embodiments Z₂ is five amino acids. In some embodiments Z₂ is LNAAS (SEQ ID NO: 386), wherein L is Leucine, N is Asparagine, A is Alanine and S is Serine. In some embodiments Z₃ is two amino acids. In some embodiments Z₃ is GT, wherein G is Glycine and T is Threonine. In some embodiments the peptide comprises ANSGLENLVILNAASLAGTGGQY (SEQ ID NO: 387).

In some embodiments the therapeutic peptide that is an α7nAChR agonist and a CLIP inhibitor has the following sequence: X₁RX₂X₃X₄X₅LX₆X₇ (SEQ ID NO: 383), wherein each X is an amino acid, wherein R is Arginine, L is Leucine and wherein at least one of X₂ and X₃ is Methionine. X refers to any amino acid, naturally occurring or modified. In some embodiments the Xs referred to the in formula X₁RX₂X₃X₄X₅LX₆X₇ (SEQ ID NO: 384) have the following values:

X₁ is Ala, Phe, Met, Leu, Be, Val, Pro, or Trp

X₂ is Ala, Phe, Met, Leu, Be, Val, Pro, or Trp

X₃ is Ala, Phe, Met, Leu, Be, Val, Pro, or Trp.

wherein X₄ is any

X₅ is Ala, Phe, Met, Leu, Be, Val, Pro, or Trp

X₆ is any

X₇ is Ala, Cys, Thr, Ser, Gly, Asn, Gln, Tyr.

The peptide preferably is FRIM X₄VLX₆S (SEQ ID NO: 388), such that X₄ and X₆ are any amino acid and may be Ala. Such a peptide is referred to as FRIMAVLAS (SEQ ID NO: 389).

The minimal peptide length for binding HLA-DR is 9 amino acids. However, there can be overhanging amino acids on either side of the open binding groove. For some well-studied peptides, it is known that additional overhanging amino acids on both the N and C termini can augment binding. Thus the peptide of SEQ ID NO 383 may be 9 amino acids in length or it may be longer. For instance, the peptide may have additional amino acids at the N and/or C terminus. The amino acids at either terminus may be anywhere between 1 and 100 amino acids. In some embodiments the peptide includes 1-50, 1-20, 1-15, 1-10, 1-5 or any integer range there between. When the peptide is referred to as “N-FRIMAVLAS-C” (SEQ ID NO: 389) or “N-X₁RX₂X₃X₄X₅LX₆X₇-C” (SEQ ID NO: 383) the -C and -N refer to the terminus of the peptide and thus the peptide is only 9 amino acids in length. However the 9 amino acid peptide may be linked to other non-peptide moieties at either the -C or -N terminus or internally.

In some embodiments the composition of α7nAChR agonist and/or CLIP inhibitor is a peptide of Table 2.

TABLE 2

| Amino Acid Sequence | SEQ ID NO. |
|---|---|
| KALVQNDTLLQVKG | 114 |
| KAMDIMNSFVNDIFERI | 115 |
| KAMGIMKSFVNDIFERI | 116 |
| KAMGNMNSFVNDIFERI | 117 |
| KAMSIMNSFVNDLFERL | 118 |
| KASGPPVSELITKA | 119 |
| KDAFLGSFLYEYSRR | 120 |
| KDDPHACYSTVFDKL | 121 |
| KEFFQSAIKLVDFQDAKA | 122 |
| KESYSVYVYKV | 123 |
| KGLVLIAFSQYLQQCPFDEHVKL | 124 |
| KHLVDEPQNLIKQ | 125 |
| KHPDSSVNFAEFSKK | 126 |
| KKQTALVELLKH | 127 |
| KKVPEVSTPTLVEVSRN | 128 |
| KLFTFHADICTLPDTEKQ | 129 |
| KLGEYGFQNALIVRY | 130 |
| KLKPDPNTLCDEFKA | 131 |
| KLVNELTEFAKT | 132 |
| KLVVSTQTALA | 133 |
| KQTALVELLKH | 134 |
| KSLHTLFGDELCKV | 135 |
| KTITLEVEPSDTIENVKA | 136 |
| KTVMENFVAFVDKC | 137 |
| KTVMENFVAFVDKCCAADDKEACFAVEGPKL | 138 |
| KTVTAMDVVYALKR | 139 |
| KVFLENVIRD | 140 |
| KVPEVSTPTLVEVSRN | 141 |
| KYLYEIARR | 142 |
| MGIMNSFVNDIFERI | 143 |
| RAGLQFPVGRV | 144 |
| RDNIQGITKPAIRR | 145 |
| REIAQDFKTDLRF | 146 |
| RFQSAAIGALQEASEAYLVGLFEDTNLCAIHAKR | 147 |
| RILGLIYEETRR | 148 |
| RISGLIYEETRG | 149 |
| RISGLIYKETRR | 150 |
| RKENHSVYVYKV | 151 |
| RLLLPGELAKH | 152 |
| RNDEELNKLLGKV | 153 |
| RNECFLSHKDDSPDLPKL | 154 |
| RRPCFSALTPDETYVPKA | 155 |
| RTLYGFGG | 156 |
| RTSKLQNEIDVSSREKS | 157 |
| RVTIAQGGVLPNIQAVLLPKK | 158 |
| LPDTEKQKL | 159 |
| YSTVFDKLK | 160 |
| ITLEVEPSD | 161 |
| LVQNDTLLQ | 162 |
| IKAMGIMKS | 163 |
| IKAMSIMNS | 164 |
| YVYKVRLLL | 165 |
| IKAMGNMNS | 166 |
| VRLLLPGEL | 167 |
| VVYALKRKV | 168 |
| YEIARRMGI | 169 |
| FRFQSAAIG | 170 |
| VVSTQTALA | 171 |
| IMNSFVNDI | 172 |
| ICTLPDTEK | 173 |
| MGIMKSFVN | 174 |
| MGIMNSFVN | 175 |
| LVELLKHKS | 176 |
| FERIKAMGI | 177 |
| FERIKAMSI | 178 |
| VLIAFSQYL | 179 |
| IMNSFVNDL | 180 |
| IMKSFVNDI | 181 |
| IQGITKPAI | 182 |
| VYVYKVRLL | 183 |
| YVYKVKGLV | 184 |
| LIYKETRRR | 185 |
| VKGLVLIAF | 186 |
| IRRREIAQD | 187 |
| VYVYKVKGL | 188 |
| VTAMDVVYA | 189 |
| YGFQNALIV | 190 |
| LVNELTEFA | 191 |
| VRYKLKPDP | 192 |
| LKTVTAMDV | 193 |
| FQNALIVRY | 194 |
| MSIMNSFVN | 195 |
| VKAKTVMEN | 196 |
| FKAKLVNEL | 197 |
| LRFRFQSAA | 198 |
| LVLIAFSQY | 199 |
| LKASGPPVS | 200 |
| VIRDKVPEV | 201 |
| VQNDTLLQV | 202 |
| MGNMNSFVN | 203 |
| YVPKARTLY | 204 |
| FQSAIKLVD | 205 |
| LYGFGGRTS | 206 |
| YKVKGLVLI | 207 |
| LVELLKHKK | 208 |
| LKHKKVPEV | 209 |
| LLKHKSLHT | 210 |
| YKVRLLLPG | 211 |
| VRNECFLSH | 212 |
| IVRYKLKPD | 213 |
| LIVRYKLKP | 214 |
| LLGKVRNEC | 215 |
| FERIKAMGN | 216 |
| VAFVDKCCA | 217 |
| LIYEETRRR | 218 |
| LIYEETRGR | 219 |
| VYALKRKVF | 220 |
| YLYEIARRM | 221 |
| LVVSTQTAL | 222 |
| VFLENVIRD | 223 |
| LVEVSRNKL | 224 |
| LIAFSQYLQ | 225 |
| IRDKVPEVS | 226 |
| LCKVKTITL | 227 |
| LIKQKHPDS | 228 |
| FERIRAGLQ | 229 |
| FQSAAIGAL | 230 |
| LVEVSRNKY | 231 |
| VKLKHLVDE | 232 |
| VYKVKGLVL | 233 |
| YALKRKVFL | 234 |
| VELLKHKKV | 235 |
| LQVKGKAMD | 236 |
| LKHKSLHTL | 237 |
| VELLKHKSL | 238 |
| VPKARTLYG | 239 |
| FKTDLRFRF | 240 |
| MDIMNSFVN | 241 |
| IKLVDFQDA | 242 |
| FVDKCKTVM | 243 |
| IHAKRRILG | 244 |
| FLYEYSRRK | 245 |
| VMENFVAFV | 246 |
| YLVGLFEDT | 247 |
| VYKVRLLLP | 248 |
| YLQQCPFDE | 249 |
| IRAGLQFPV | 250 |
| LLKHKKVPE | 251 |
| IKQKHPDSS | 252 |
| VLPNIQAVL | 253 |
| VEPSDTIEN | 254 |
| FGGRTSKLQ | 255 |
| VAFVDKCKT | 256 |
| FFQSAIKLV | 257 |
| FQDAKAKES | 258 |
| IQAVLLPKK | 259 |
| LLQVKGKAM | 260 |
| IAFSQYLQQ | 261 |
| FLGSFLYEY | 262 |
| FVNDIFERI | 263 |
| VDEPQNLIK | 264 |
| LSHKDDSPD | 265 |
| FLSHKDDSP | 266 |
| LPNIQAVLL | 267 |
| LKRKVFLEN | 268 |
| LLPGELAKH | 269 |
| FVAFVDKCC | 270 |
| IFERIKAMS | 271 |
| IENVKAKTV | 272 |
| VSRNKLFTF | 273 |
| LKPDPNTLC | 274 |
| MENFVAFVD | 275 |
| YSRRKDDPH | 276 |
| LFGDELCKV | 277 |
| FERLKASGP | 278 |
| VSTQTALAK | 279 |
| FAKTKLVVS | 280 |
| VTIAQGGVL | 281 |
| LNKLLGKVR | 282 |
| LYEIARRMG | 283 |
| MKSFVNDIF | 284 |
| LFTFHADIC | 285 |
| LAKQTALVE | 286 |
| FVAFVDKCK | 287 |
| FVNDLFERL | 288 |
| VKTITLEVE | 289 |
| IAQGGVLPN | 290 |
| LRRPCFSAL | 291 |
| LGSFLYEYS | 292 |
| LCAIHAKRR | 293 |
| LPKLRRPCF | 294 |
| VEVSRNKLF | 295 |
| FLENVIRDK | 296 |
| IYKETRRRK | 297 |
| VEVSRNKYL | 298 |
| FVDKCCAAD | 299 |
| LFEDTNLCA | 300 |
| VNFAEFSKK | 301 |
| VGRVRDNIQ | 302 |
| MNSFVNDIF | 303 |
| MNSFVNDLF | 304 |
| LVDEPQNLI | 305 |
| FSKKKKQTA | 306 |
| YGFGGRTSK | 307 |
| LITKAKDAF | 308 |
| MDVVYALKR | 309 |
| LLLPGELAK | 310 |
| LQFPVGRVR | 311 |
| LKEFFQSAI | 312 |
| YEYSRRKDD | 313 |
| LTPDETYVP | 314 |
| LGKVRNECF | 315 |
| LKHLVDEPQ | 316 |
| LQNEIDVSS | 317 |
| LVDFQDAKA | 318 |
| FAVEGPKLK | 319 |
| VSELITKAK | 320 |
| IFERIRAGL | 321 |
| LENVIRDKV | 322 |
| VGLFEDTNL | 323 |
| VSSREKSRV | 324 |
| IYEETRRRI | 325 |
| IFERIKAMG | 326 |
| FGDELCKVK | 327 |
| LFERLKASG | 328 |
| IARRMGIMN | 329 |
| LGLIYEETR | 330 |
| ILGLIYEET | 331 |
| YEETRRRIS | 332 |
| IDVSSREKS | 333 |
| LHTLFGDEL | 334 |
| LVGLFEDTN | 335 |
| VKGKAMDIM | 336 |
| FPVGRVRDN | 337 |
| VSRNKYLYE | 338 |
| IAQDFKTDL | 339 |
| FHADICTLP | 340 |
| VRDNIQGIT | 341 |
| YKLKPDPNT | 342 |
| VDFQDAKAK | 343 |
| FAEFSKKKK | 344 |
| LYEYSRRKD | 345 |
| FDEHVKLKH | 346 |
| LTEFAKTKL | 347 |
| LQQCPFDEH | 348 |
| LEVEPSDTI | 349 |
| IGALQEASE | 350 |
| VDKCKTVME | 351 |
| VFDKLKEFF | 352 |
| FTFHADICT | 353 |
| VPEVSTPTL | 354 |
| FSALTPDET | 355 |
| ITKPAIRRR | 356 |
| YKETRRRKE | 357 |
| IYEETRGRI | 358 |
| VEGPKLKTV | 359 |
| FEDTNLCAI | 360 |
| VNELTEFAK | 361 |
| YSVYVYKVK | 362 |
| LQEASEAYL | 363 |
| ISGLIYKET | 364 |
| YEETRGRIS | 365 |
| FDKLKEFFQ | 366 |
| VSTPTLVEV | 367 |
| VNDLFERLK | 368 |
| LPGELAKHR | 369 |
| VNDIFERIK | 370 |
| FSQYLQQCP | 371 |
| ITKAKDAFL | 372 |
| LGEYGFQNA | 373 |
| LCDEFKAKL | 374 |
| VDKCCAADD | 375 |
| VNDIFERIR | 376 |
| ISGLIYEET | 377 |
| LAKHRNDEE | 378 |

In some embodiments, the peptides have attached amino acid residues comprising “flanking sequences”, such as ANSG.

A composition of α7nAChR agonist and/or CLIP inhibitor may include one or more of the peptides listed in Table 1. In some embodiments the composition of α7nAChR agonist and/or CLIP inhibitor may include one or more of the peptides listed in Table 2.

The invention also involves the discovery of various subsets of the therapeutic peptides of the invention based on the ability of the inhibitor to bind to α7nAChR and/or MHC class I or II generally or even to individual specific MHC.

The peptide may be cyclic or non-cyclic. Cyclic peptides in some instances have improved stability properties. Those of skill in the art know how to produce cyclic peptides.

The peptides may also be linked to other molecules. The two or more molecules may be linked directly to one another (e.g., via a peptide bond); linked via a linker molecule, which may or may not be a peptide; or linked indirectly to one another by linkage to a common carrier molecule, for instance.

Thus, linker molecules (“linkers”) may optionally be used to link the peptide to another molecule. Linkers may be peptides, which consist of one to multiple amino acids, or non-peptide molecules. Examples of peptide linker molecules useful in the invention include glycine-rich peptide linkers (see, e.g., U.S. Pat. No. 5,908,626), wherein more than half of the amino acid residues are glycine. Preferably, such glycine-rich peptide linkers consist of about 20 or fewer amino acids.

Linker molecules may also include non-peptide or partial peptide molecules. For instance, the peptide may be linked to other molecules using well known cross-linking molecules such as glutaraldehyde or EDC (Pierce, Rockford, Ill.). Bifunctional cross-linking molecules are linker molecules that possess two distinct reactive sites. For example, one of the reactive sites of a bifunctional linker molecule may be reacted with a functional group on a peptide to form a covalent linkage and the other reactive site may be reacted with a functional group on another molecule to form a covalent linkage. General methods for cross-linking molecules have been reviewed (see, e.g., Means and Feeney, Bioconjugate Chem., 1: 2-12 (1990)).

Homobifunctional cross-linker molecules have two reactive sites which are chemically the same. Examples of homobifunctional cross-linker molecules include, without limitation, glutaraldehyde; N,N′-bis(3-maleimido-propionyl-2-hydroxy-1,3-propanediol (a sulfhydryl-specific homobifunctional cross-linker); certain N-succinimide esters (e.g., discuccinimyidyl suberate, dithiobis (succinimidyl propionate), and soluble bis-sulfonic acid and salts thereof (see, e.g., Pierce Chemicals, Rockford, Ill.; Sigma-Aldrich Corp., St. Louis, Mo.).

Preferably, a bifunctional cross-linker molecule is a heterobifunctional linker molecule, meaning that the linker has at least two different reactive sites, each of which can be separately linked to a peptide or other molecule. Use of such heterobifunctional linkers permits chemically separate and stepwise addition (vectorial conjunction) of each of the reactive sites to a selected peptide sequence. Heterobifunctional linker molecules useful in the invention include, without limitation, m-maleimidobenzoyl-N-hydroxysuccinimide ester (see, Green et al., Cell, 28: 477-487 (1982); Palker et al., Proc. Natl. Acad. Sci (USA), 84: 2479-2483 (1987)); m-maleimido-benzoylsulfosuccinimide ester; γ-maleimidobutyric acid N-hydroxysuccinimide ester; and N-succinimidyl 3-(2-pyridyl-dithio)propionate (see, e.g., Carlos et al., Biochem. J., 173: 723-737 (1978); Sigma-Aldrich Corp., St. Louis, Mo.).

The carboxyl terminal amino acid residue of the peptides described herein may also be modified to block or reduce the reactivity of the free terminal carboxylic acid group, e.g., to prevent formation of esters, peptide bonds, and other reactions. Such blocking groups include forming an amide of the carboxylic acid group. Other carboxylic acid groups that may be present in polypeptide may also be blocked, again provided such blocking does not elicit an undesired immune reaction or significantly alter the capacity of the peptide to specifically function.

The peptide for instance, may be linked to a PEG molecule. Such a molecule is referred to as a PEGylated peptide.

The invention provides therapeutic peptides which can be purified or synthesized. Thus, it would be valuable if the structure of other therapeutic peptides or fragments thereof may be predicted based on the amino acid sequences provided herein. Structure prediction, analysis of crystallographic data, sequence alignment, as well as homology modeling, can be accomplished using computer software programs available in the art, such as BLAST, CHARMm release 21.2 for the Convex, and QUANTA v. 3.3, (Molecular Simulations, Inc., York, United Kingdom).

The invention further provides derivatives (including but not limited to fragments), and analogs of the therapeutic peptides set forth in Table 1. The production and use of derivatives and analogs related to therapeutic peptide are within the scope of the present invention.

In particular, therapeutic peptide derivatives can be made by altering the therapeutic peptide sequences disclosed herein by substitutions, insertions or deletions that provide for functionally equivalent molecules. The therapeutic peptide derivatives of the invention include, but are not limited to, those containing, as a primary amino acid sequence, all or part of the amino acid sequence of a therapeutic peptide including altered sequences in which functionally equivalent amino acid residues are substituted for residues within the sequence resulting in a silent change (i.e., conservative substitutions). For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. therapeutic peptide derivatives of the invention also include, but are not limited to, those containing, as a primary amino acid sequence, all or part of the amino acid sequence of a therapeutic peptide including altered sequences in which amino acid residues are substituted for residues with similar chemical properties (i.e., conservative substitutions). In specific embodiments, 1, 2, 3, 4, or 5 amino acids are substituted.

Derivatives or analogs of therapeutic peptide include, but are not limited to, those peptides which are substantially homologous to therapeutic peptide or fragments thereof.

Included within the scope of the invention are therapeutic peptide fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, reagents useful for protection or modification of free NH₂-groups, free COOH-groups, OH-groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH₄; acetylation, formylation, oxidation, reduction; metabolic synthesis in the presence of tunicamycin; etc.

Furthermore, if desired, nonclassical amino acids or chemical amino acid analogs can be introduced as a substitution or addition into the therapeutic peptide sequence. Nonclassical amino acids include, but are not limited to, the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, and Nα-methyl amino acids.

In a specific embodiment, the therapeutic peptide derivative is a chimeric, or fusion, protein comprising a therapeutic peptide fused via a peptide bond at its amino- and/or carboxy-terminus to an alternative peptide. In an embodiment, the alternative peptide is fused at the amino-terminus of a therapeutic peptide. In one embodiment, such a chimeric protein is produced by recombinant expression or by protein synthetic techniques, e.g., by use of a peptide synthesizer. In a specific embodiment, such chimeric construction can be used to enhance one or more desired properties of a therapeutic peptide, including but not limited to, stability, solubility, or resistance to proteases. In another embodiment, chimeric construction can be used to target therapeutic peptide to a specific site, e.g., a chimeric construction comprising a therapeutic peptide fused to an antibody to a specific type of tissue allows therapeutic peptide to be delivered to the tissue site.

The therapeutic peptide sequence can be characterized by a hydrophilicity analysis (Hopp, T. and Woods, K., 1981, Proc. Natl. Acad. Sci. U.S.A. 78: 3824). A hydrophilicity profile can be used to identify the hydrophobic and hydrophilic regions of the therapeutic peptide.

Secondary structural analysis (Chou, P. and Fasman, G., 1974, Biochemistry 13: 222) can also be done, to identify regions of the therapeutic peptide that assume specific secondary structures.

The peptides useful herein are isolated peptides. As used herein, the term “isolated” means that the referenced material is removed from its native environment, e.g., a cell. Thus, an isolated biological material can be free of some or all cellular components, i.e., components of the cells in which the native material is occurs naturally (e.g., cytoplasmic or membrane component). The isolated peptides may be substantially pure and essentially free of other substances with which they may be found in nature or in vivo systems to an extent practical and appropriate for their intended use. Typically the isolated peptides are synthetic. In particular, the peptides are sufficiently pure and are sufficiently free from other biological constituents of their hosts cells so as to be useful in, for example, producing pharmaceutical preparations or sequencing. Because an isolated peptide of the invention may be admixed with a pharmaceutically acceptable carrier in a pharmaceutical preparation, the peptide may comprise only a small percentage by weight of the preparation. The peptide is nonetheless substantially pure in that it has been substantially separated from at least one of the substances with which it may be associated in living systems.

The term “purified” in reference to a protein or a nucleic acid, refers to the separation of the desired substance from contaminants to a degree sufficient to allow the practitioner to use the purified substance for the desired purpose. Preferably this means at least one order of magnitude of purification is achieved, more preferably two or three orders of magnitude, most preferably four or five orders of magnitude of purification of the starting material or of the natural material. In specific embodiments, a purified therapeutic peptide is at least 60%, at least 80%, or at least 90% of total protein by weight. In a specific embodiment, a purified therapeutic peptide is purified to homogeneity as assayed by, e.g., sodium dodecyl sulfate polyacrylamide gel electrophoresis, or agarose gel electrophoresis.

The instant invention is based at least in part on the discovery that specific peptides are α7nAChR agonists and are useful in the methods of the invention. The invention, thus, involves treatments for psychiatric and neurological disease, chronic wounds, cancer, autoimmune disease, Alzheimer's disease and transplant rejection as well as others by administering to a subject in need thereof a therapeutic α7nAChR agonist peptide.

A subject shall mean a human or vertebrate mammal including but not limited to a dog, cat, horse, goat and primate, e.g., monkey. Thus, the invention can also be used to treat diseases or conditions in non-human subjects. Preferably the subject is a human.

As used herein, the term treat, treated, or treating when used with respect to a disorder refers to a prophylactic treatment which increases the resistance of a subject to development of the disease or, in other words, decreases the likelihood that the subject will develop the disease as well as a treatment after the subject has developed the disease in order to fight the disease, prevent the disease from becoming worse, or slow the progression of the disease compared to in the absence of the therapy.

When used in combination with the therapies of the invention the dosages of known therapies may be reduced in some instances, to avoid side effects.

The therapeutic peptide can be administered in combination with other therapeutic agents and such administration may be simultaneous or sequential. When the other therapeutic agents are administered simultaneously they can be administered in the same or separate formulations, but are administered at the same time. The administration of the other therapeutic agent and the therapeutic peptide can also be temporally separated, meaning that the therapeutic agents are administered at a different time, either before or after, the administration of the therapeutic peptide. The separation in time between the administration of these compounds may be a matter of minutes or it may be longer.

For instance, the therapeutic peptide may be administered in combination with an antibody such as an anti-MHC antibody. The purpose of exposing a cell to an anti-MHC class II antibody, for instance in some embodiments, is to prevent the cell, once CLIP has been removed, from picking up a self-antigen, which could be presented in the context of MHC, if the cell does not pick up the therapeutic peptide right away. An anti-MHC class II antibody may also engage a B cell and kill it. Once CLIP has been removed, the antibody will be able to interact with the MHC and cause the B cell death. This prevents the B cell with an empty MHC from picking up and presenting self-antigen or from getting another CLIP molecule in the surface that could lead to further γδ T cell expansion and activation. Additionally, the therapeutic peptide may be used in combination with an immunotherapy that may have the possibility of inducing a cytokine storm, such as checkpoint inhibitors or chimeric antigen receptor expressing cells, in order to dampen the systemic immune response.

According to an embodiment of the invention, the methods described herein are useful in transplant procedures. Thus, the methods are useful for such grafted tissue as heart, lung, kidney, skin, cornea, liver, neuronal tissue or cell, or with stem cells, including hematopoietic or embryonic stem cells, for example.

The success of surgical transplantation of organs and tissue is largely dependent on the ability of the clinician to modulate the immune response of the transplant recipient. Specifically the immunological response directed against the transplanted foreign tissue must be controlled if the tissue is to survive and function. Currently, skin, kidney, liver, pancreas, lung and heart are the major organs or tissues with which allogeneic transplantations are performed. It has long been known that the normally functioning immune system of the transplant recipient recognizes the transplanted organ as “non-self” tissue and thereafter mounts an immune response to the presence of the transplanted organ. Left unchecked, the immune response will generate a plurality of cells and proteins that will ultimately result in the loss of biological functioning or the death of the transplanted organ.

This tissue/organ rejection can be categorized into three types: hyperacute, acute and chronic. Hyperacute rejection is essentially caused by circulating antibodies in the blood that are directed against the tissue of the transplanted organ (transplant). Hyperacute rejection can occur in a very short time and leads to necrosis of the transplant. Acute graft rejection reaction is also immunologically mediated and somewhat delayed compared to hyperacute rejection. The chronic form of graft rejection that can occur years after the transplant is the result of a disease state commonly referred to as Graft Arterial Disease (GAD). GAD is largely a vascular disease characterized by neointimal proliferation of smooth muscle cells and mononuclear infiltrates in large and small vessels. This neointimal growth can lead to vessel fibrosis and occlusion, lessening blood flow to the graft tissue and resulting in organ failure. Current immunosuppressant therapies do not adequately prevent chronic rejection. Most of the gains in survival in the last decade are due to improvements in immunosuppressive drugs that prevent acute rejection. However, chronic rejection losses remain the same and drugs that can prevent it are a critical unmet medical need.

In a transplant situation, the organ tissue recipient may be treated with an agonist described herein to prevent the recipients body from mounting an immune response, thus, helping ward off life-threatening immune attack. The methods of treating transplant/graft rejection can be applied in conjunction with, or supplementary to, the customary treatments of transplant/graft rejection. Tissue graft and organ transplant recipients are customarily treated with one or more cytotoxic agents in an effort to suppress the transplant recipient's immune response against the transplanted organ or tissue.

According to an embodiment of the invention, the methods described herein are useful in inhibiting the development of an autoimmune disease in a subject by administering a therapeutic peptide to the subject. Thus, the methods are useful for such autoimmune diseases as multiple sclerosis, systemic lupus erythematosus, type 1 diabetes, viral endocarditis, viral encephalitis, rheumatoid arthritis, Graves' disease, autoimmune thyroiditis, autoimmune myositis, and discoid lupus erythematosus.

In some embodiments, the present invention provides a method of treating a cancer comprising administering to a subject in whom such treatment is desired a therapeutically effective amount of a composition comprising a therapeutic peptide. A composition of the invention may, for example, be used as a first, second, third or fourth line cancer treatment. In some embodiments, the invention provides methods for treating a cancer (including ameliorating a symptom thereof) in a subject refractory to one or more conventional therapies for such a cancer, said methods comprising administering to said subject a therapeutically effective amount of a composition comprising a therapeutic peptide. A cancer may be determined to be refractory to a therapy when at least some significant portion of the cancer cells are not killed or their cell division are not arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced, or has increased.

The invention, in some aspects, provides methods for treating a cancer (including ameliorating one or more symptoms thereof) in a subject refractory to existing single agent therapies for such a cancer, said methods comprising administering to said subject a therapeutically effective amount of a composition comprising a therapeutic peptide and a therapeutically effective amount of one or more therapeutic agents other than the therapeutic peptide. The invention also provides methods for treating cancer by administering a composition comprising a therapeutic peptide in combination with any other anti-cancer treatment (e.g., checkpoint inhibitors, CAR-T cells, radiation therapy, chemotherapy or surgery) to a patient who has proven refractory to other treatments or who could benefit from adjunct therapy. The invention also provides methods for the treatment of a patient having cancer and immunosuppressed by reason of having previously undergone one or more other cancer therapies. The invention also provides alternative methods for the treatment of cancer where chemotherapy, radiation therapy, hormonal therapy, and/or biological therapy/immunotherapy has proven or may prove too toxic, i.e., results in unacceptable or unbearable side effects, for the subject being treated.

Cancers that can be treated by the methods encompassed by the invention include, but are not limited to, neoplasms, malignant tumors, metastases, or any disease or disorder characterized by uncontrolled cell growth such that it would be considered cancerous. The cancer may be a primary or metastatic cancer.

Cancers include, but are not limited to, biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer.

The compositions of the invention also can be administered to prevent progression to a neoplastic or malignant state. Such prophylactic use is indicated in conditions known or suspected of preceding progression to neoplasia or cancer, in particular, where non-neoplastic cell growth consisting of hyperplasia, metaplasia, or most particularly, dysplasia has occurred. Hyperplasia is a form of controlled cell proliferation involving an increase in cell number in a tissue or organ, without significant alteration in structure or function. Endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of the composition of the invention. Such characteristics of a transformed phenotype include morphology changes, looser substratum attachment, loss of contact inhibition, loss of anchorage dependence, protease release, increased sugar transport, decreased serum requirement, expression of fetal antigens.

In other embodiments, a patient which exhibits one or more of the following predisposing factors for malignancy is treated by administration of an effective amount of a composition of the invention: a chromosomal translocation associated with a malignancy (e.g., the Philadelphia chromosome for chronic myelogenous leukemia, t(14; 18) for follicular lymphoma, etc.), familial polyposis or Gardner's syndrome (possible forerunners of colon cancer), benign monoclonal gammopathy (a possible forerunner of multiple myeloma), a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2d Ed., W.B. Saunders Co., Philadelphia, pp. 112-113) etc.), and exposure to carcinogens (e.g., smoking, and inhalation of or contacting with certain chemicals).

In one set of embodiments, the invention includes a method of treating a subject susceptible to or exhibiting symptoms of cancer. The cancer may be primary, metastatic, recurrent or multi-drug resistant. In some cases, the cancer is drug-resistant or multi-drug resistant. As used herein, a “drug-resistant cancer” is a cancer that is resistant to conventional commonly-known cancer therapies. Examples of conventional cancer therapies include treatment of the cancer with agents such as methotrexate, trimetrexate, adriamycin, taxotere, doxorubicin, 5-flurouracil, vincristine, vinblastine, pamidronate disodium, anastrozole, exemestane, cyclophosphamide, epirubicin, toremifene, letrozole, trastuzumab, megestrol, tamoxifen, paclitaxel, docetaxel, capecitabine, goserelin acetate, etc. A “multi-drug resistant cancer” is a cancer that resists more than one type or class of cancer agents, i.e., the cancer is able to resist a first drug having a first mechanism of action, and a second drug having a second mechanism of action.

The α7nAChR agonists may be administered in combination with other anti-cancer agents such as checkpoint inhibitors.

Inhibitory checkpoint molecules include, but are not limited to: PD-1, PD-L1, PD-L2, TIM-3, VISTA, A2AR, B7-H3, B7-H4, B7-H6, BTLA, CTLA-4, IDO, KIR and LAG3. CTLA-4, PD-1, and ligands thereof are members of the CD28-B7 family of co-signaling molecules that play important roles throughout all stages of T-cell function and other cell functions. CTLA-4, Cytotoxic T-Lymphocyte-Associated protein 4 (CD152), is involved in controlling T cell proliferation.

The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and inhibits it. Cancer cells take advantage of this system by driving high levels of expression of PD-L1 on their surface. This allows cancer cells to gain control of the PD-1 pathway and switch off T cells expressing PD-1 that may enter the tumor microenvironment, thus suppressing the anticancer immune response. Pembrolizumab (formerly MK-3475 and lambrolizumab, trade name KEYTRUDA®) is a human antibody used in cancer immunotherapy and targets the PD-1 receptor.

The checkpoint inhibitor, in some embodiments, is a molecule such as a monoclonal antibody, a humanized antibody, a fully human antibody, a fusion protein or a combination thereof or a small molecule. For instance, the checkpoint inhibitor inhibits a checkpoint protein which may be CTLA-4, PDL1, PDL2, PD1, B7-H3, B7-H4, B7-H6, BTLA, HVEM, TIM3, GAL9, LAG3, VISTA, KIR, 2B4, CD160, CGEN-15049, CHK 1, CHK2, A2aR, B-7 family ligands or a combination thereof. Ligands of checkpoint proteins include but are not limited to CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands. In some embodiments the anti-PD-1 antibody is BMS-936558 (nivolumab). In other embodiments the anti-CTLA-4 antibody is ipilimumab (trade name Yervoy, formerly known as MDX-010 and MDX-101). In another embodiment, the checkpoint inhibitor is J43 (an anti-PD1 antibody), RMP1-14 (an anti-PD1 antibody), or atezolizumab (TECENTRIQ®; an anti-PDL1 antibody). In yet other embodiments, the checkpoint inhibitor is pembrolizumab.

Pembrolizumab is a potent humanized immunoglobulin G4 monoclonal antibody with high specificity of binding to the PD-1 receptor, thus inhibiting its interaction with PD-L1 and programmed cell death 1 ligand 2. Based on preclinical in vitro data, pembrolizumab has high affinity and potent receptor blocking activity for PD-1. Pembrolizumab has an acceptable preclinical safety profile and is in clinical development as an IV immunotherapy for advanced malignancies. KEYTRUDA® (pembrolizumab) is approved for the treatment of patients across a number of indications. Pembrolizumab is approved for use in several cancer types, and is under investigation in several phases of clinical development for many more.

In some embodiments, the compositions and methods further comprise administering at least one immune checkpoint inhibitor, as described herein. In some embodiments, combinations of immune checkpoint inhibitors are administered.

The α7nAChR agonists may be administered in combination with cellular therapy such as CAR-T cells. A CAR-T cell, as used herein, refers to T cells into which a chimeric antigen receptor has been introduced to redirect the receptor specificity towards an antigen of choice. Such receptors comprise an ectodomain that recognizes antigen independent of MHC restriction, in combination with cytoplasmic signaling domains. Many different peptides can be introduced into the T cells as the ectodomain of the chimeric antigen receptors. Examples include a nanobody, a monoclonal antibody, a humanized antibody, a chimeric antibody, a human antibody, or an antibody fragment.

The CAR-T cell may be a cell (e.g., T cell) engineered to express a CAR wherein the CAR-T cell exhibits an antitumor property. The CAR can be engineered to comprise an ectodomain peptide fused to an intracellular signaling domain of the T cell antigen receptor complex Zeta chain (e.g., CD3 Zeta). The CAR when expressed in a T cell is able to redirect antigen recognition based on the antigen binding specificity. The antigen binding peptide is preferably fused with an intracellular domain from one or more of a costimulatory molecule and a Zeta chain. In some embodiments, the antigen binding peptide is fused with one or more intracellular domains selected from the group of a CD137 (4-1BB) signaling domain, a CD28 signaling domain, a CD3 Zeta signal domain, and any combination thereof.

Another form of anti-cancer therapy involves administering an antibody specific for a cell surface antigen of, for example, a cancer cell. In one embodiment, the antibody may be selected from the group consisting of Ributaxin, Herceptin, Rituximab, Quadramet, Panorex, IDEC-Y2B8, BEC2, C225, Oncolym, SMART M195, ATRAGEN, Ovarex, Bexxar, LDP-03, for t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, BABS, anti-FLK-2, MDX-260, ANA Ab, SMART 1D10 Ab, SMART ABL 364 Ab and ImmuRAIT-CEA. Other antibodies include but are not limited to anti-CD20 antibodies, anti-CD40 antibodies, anti-CD19 antibodies, anti-CD22 antibodies, anti-HLA-DR antibodies, anti-CD80 antibodies, anti-CD86 antibodies, anti-CD54 antibodies, and anti-CD69 antibodies. These antibodies are available from commercial sources or may be synthesized de novo.

Other cancer therapies and their dosages, routes of administration and recommended usage are known in the art and have been described in such literature.

Traumatic brain injuries (TBI) occur at a rate of greater than 2 million per year, and the serious clinical problems that occur after TBI affect approximately 5 million people in the U.S. alone. Currently, there are no effective therapies. It has been discovered that the immune system may directly contribute to inflammation and degeneration of the brain, and behavioral deficits following TBI. CD74, a protein involved in both the innate and adaptive immune responses, may contribute to acute, early and chronic pathologies that result from TBI.

Over 5 million Americans suffer from Alzheimer's disease (AD) and related dementias (ADRD). According to the CDC, by 2060, the burden of AD and ADRD is projected to grow to 13.9 million people in the United States, which amounts to an alarming 3.3 percent of the population. Despite intensive research efforts, disease mechanisms, diagnostic tools, and therapies remain elusive. While genetic mutations account for a small proportion of ADRD, the most common risk factor for dementia is prior central nervous system (CNS) injury. Suffering from a traumatic brain injury (TBI) results in mild cognitive impairment (MI) in most patients, and a mild to moderate TBI increases the risk for AD/ADRDs 2.3-fold, and a severe TBI increases the risk 4.5-fold

The α7nAChR agonists of the invention are also useful in treating Alzheimer's disease (AD). AD is a degenerative brain disorder characterized by cognitive and noncognitive neuropsychiatric symptoms, which accounts for approximately 60% of all cases of dementia for patients over 65 years old. Psychiatric symptoms are common in AD, with psychosis (hallucinations and delusions) present in many patients.

There are currently no effective treatments for AD and there is a critical unmet clinical need for therapies. While genetic mutations account for a small proportion of early onset AD, the most common risk factor for dementia is prior central nervous system (CNS) injury. Suffering from a traumatic brain injury (TBI) results in mild cognitive impairment (MI) in most patients, and a mild to moderate TBI increases the risk 2.3-fold for AD and AD-related disorders (ADRD), and a severe TBI increases the risk 4.5-fold. Thus, TBI is a major risk factor for the development of MI/AD/ADRD.

Inflammation, both innate and adaptive, are widely thought to play a major role in the pathogenesis of MI/AD/ADRD. Despite extensive preclinical and clinical investments, supported by a vast literature, innate and adaptive immune mechanisms of MI/AD/ADRD remain elusive. Amyloid beta, is known to bind to toll-like receptors (TLRs) on microglial cells, among others, stimulating the MyD88 complex, resulting in the expansion of pro-inflammatory cells contributing to the immune response. Innate immunity and inflammation precede an adaptive immune response and there is accumulating evidence that adaptive immunity, including T cell activation and expansion, contribute to AD pathology. A recent report demonstrated an immune signature associated with AD, involving peripheral and CNS expansion of a subset of CD8+ T cells, CD45RA+CD8+T effector memory cells. Expansion of these cells is indicative of adaptive immunity, and novel mechanistic approaches are needed to identify putative immune targets to the adaptive response in AD.

Applicant has discovered that MHC class II invariant peptide, CLIP, contributes to TBI-induced neuropathology and neurobehavioral deficits. The therapeutic peptides that antagonize CLIP-binding to the MHC class II antigen-binding groove, described herein, prevent and/or misguide T cell recognition of antigens presented by MHC class II, and have profound anti-inflammatory effects after a TBI, that is accompanied by neuroprotection.

The α7nAChR agonists are useful in treating psychiatric disease or neurological disease. In some embodiments the psychiatric or neurological disease is selected from the group consisting of: schizophrenia, mania, depression, and anxiety. In other embodiments the psychiatric or neurological disease is selected from the group consisting of attention deficit hyperactivity disorder (ADHD), Parkinson's Disease, PANS, PANDAS, Huntington's chorea, epilepsy, convulsions, Tourette syndrome, obsessive compulsive disorder (OCD), memory deficits and dysfunction, a learning deficit, a panic disorder, narcolepsy, nociception, autism, schizophrenia, tardive dyskinesia, social phobia, pseudo dementia neuropathic pain, postoperative pain, inflammatory pain, and phantom limb pain.

In yet other embodiments the psychiatric or neurological disease is a neurodegenerative disorder. Many neurological diseases involve neurodegeneration. For instance, Alzheimer's, Parkinson's disease, traumatic brain injury (TBI), ALS, Prion disease, Motor neurone diseases (MND), Huntington's disease (HD), Spinocerebellar ataxia (SCA), Spinal muscular atrophy (SMA), Picks Disease, Chronic lead poisoning, Rett Disease, Neuronal curoid lipofuscinosis, Metachromatic leukodystrophy, Alexander disease, Canavan disease, Schilder's Disease, Devic Disease, Friedrich's Ataxia, Ataxia telangiectasia,

Wilson's disease, Niemann-Pick disease, Tay-Sachs Disease, Krabbe disease, Gaucher disease, Adrenoleukodystrophy, dementia and an intellectual impairment disorder.

Neurodegeneration, the loss of neurons, is at the core of diseases like Parkinson's Disease, Alzheimer's Disease, and post-TBI syndromes. The neurons are the primary cells of the central nervous system and provide the essential functions of communicating with other local neurons or with innervated tissues via the synaptic release of neurotransmitters. Neurotransmitter release then delivers excitatory or inhibitory signals that result in an action or inhibition of action. Neurodegeneration disturbs these connections and neurodegeneration has been shown to systematically progress once the process begins. To date, no therapeutic interventions have been identified that can interrupt the cascade of events involved in progressing neurodegeneration. (Ransohoff, R. M. How Neuroinflammation Contributes to Neurodegeneration. Science. 2016. 353: (6301), 777-779.)

Parkinson's Disease (PD) is a complex neurological disorder characterized by both motor and non-motor symptoms. As a movement disorder, PD involves motor symptoms including bradykinesia (slowed movements), muscle rigidity, tremors at rest, and postural/gait impairment. The motor symptoms are associated with Lewy bodies and death of dopaminergic neurons in the substantia nigra. The non-motor features, such as olfactory dysfunction, cognitive impairment, psychiatric symptoms, sleep disorders, autonomic dysfunction, pain, and fatigue, involve extensive regions of the nervous system, multiple neurotransmitter pathways, and protein aggregation, including aggregation of alpha-synuclein. Once viewed as primarily a disease associated with environmental factors, the cause of PD remains unknown, but disease-associated risk factors now include a growing list of genetic, immunologic, and environmental factors. There are also significant clinical challenges in treatment for PD that include the difficulty in making a definitive diagnosis early in the disease course and the complexity and difficulty of managing disease symptoms as the disease progresses. There have been no discoveries to date that can slow the neurodegenerative process (Kalia, L V; Lang, A E. Parkinson's Disease. Lancet 2015; 386: 896-912). The central nervous system (CNS) is protected by the skull, the spine, and the meninges. The BBB is a part of the meninges and provides a selective filter and barrier that protects the brain. The BBB provides a tightly regulated exchange between molecules and cells in the blood stream and the CNS. Importantly, the BBB confers protection from pathogens. The BBB consists of vessels formed of continuous endothelial cells that have proteins known as “tight junction” proteins that provide for selective intercellular boundaries that restrict the passage of molecules and of certain cells from entering the brain parenchyma. The BBB also restricts the entry and trafficking of host immune cells, such as T cells and B cells, into the brain. Thus, host defenses in the brain are usually limited to innate immune cells that include microglia and non-parenchymal macrophages.

BBB permeability is a well-recognized characteristic of PD. In fact, misfolded/aggregated alpha-synuclein has been implicated in the neurodegeneration and neuroinflammation via the activation of microglia and astrocytes in the brain. As discussed above, the therapeutic peptides disclosed herein activate the cholinergic anti-inflammatory response, targeting pro-inflammatory cells, restoring BBB integrity and reducing permeability. PD patients have higher numbers of T cells in the ventral midbrain than healthy controls. Recent studies indicate that the brain-infiltrating T cells may be autoreactive and may recognize an important contributor to PD, the protein α-synuclein. To date, the immune contributions of α-synuclein to PD are known to include: (1) the fact that aggregates of α-synuclein bind to and activate Toll-like Receptor (TLR)-driven acute inflammatory responses; and (2) evidence that CD4 T cells from PD patients recognize and respond to synuclein peptides. Based, on the discoveries disclosed herein, a new therapeutic intervention for the treatment of PD is provided. PD may be treated using the therapeutic peptides of the invention.

The therapeutic peptides of the invention are also useful for targeting the cholinergic anti-inflammatory pathway and the immune response as a target for Pediatric Acute-onset Neuropsychiatric Syndrome (PANS). PANS is a childhood disorder that is characterized by the sudden onset of symptoms that include obsessive compulsive disorder (OCD), eating restriction, and acute behavioral deterioration. PANS is a clinically defined syndrome that does not require a known trigger, although it is frequently associated with exposure to pathogens. Pediatric Autoimmune Neuropsychiatric Disorders Associated with Streptococcal Infections (PANDAS) is a condition that is a subset of PANS that is associated with streptococcal infections. PANDAS was first reported by a team of investigators from the National Institute of Mental Health, a section of the National Institute of Health (NIH). There are 5 criteria for diagnosing PANDAS, including an abrupt onset of OCD, disabling tics, a relapsing remitting pathology, pediatric onset, and a temporal association with exposure to Group A Streptococcus (GAS).

Streptococcus pyogenes (including Group A Strep or GAS) infection is associated with multiple autoimmune conditions, including rheumatic fever, Sydenham's chorea (a movement disorder), and PANDAS. The incidence of strep infection among children is very high, but the incidence of the associated autoimmune syndromes is low relative to the number of strep infections. Several factors likely account for this dichotomy, including genetic predisposition, the number of repetitive infections, a potential breach in the integrity of the blood brain barrier (BBB), and other environmental risk factors, including the nature of the immune response when the infection is left untreated. The primary theory for how strep and other bacteria can result in post-infectious autoimmunity is one called “molecular mimicry” in which an immune response to streptococcal antigens is “cross reactive” against antigens that are part of host tissues, such as antibodies that were originally formed in response to strep, but that also recognize and activate or inactivate important “self-receptors” in the heart, the brain, or elsewhere. In rheumatic fever, antibodies to strep are thought to recognize self-antigens in the heart, leading to destructive damage to the heart. Similarly, in PANDAS, antibodies produced in response to strep infection are thought to recognize brain antigens.

Multiple sclerosis (MS), amyotrophic lateral sclerosis (ALS), epilepsy and post-infectious neurological disorders, including PANDAS and post-infections epilepsy are all examples of autoimmune neuropsychiatric disorders with demonstrated BBB dysfunction (e.g. increased permeability) that facilitates peripheral immune cell migration or antibody extravasation into the brain resulting in the neuropathology.

Using a mouse model in which mice are infected intranasally with GAS, scientists have examined the nasal associated lymphoid tissue (NALT) and have found that repetitive intranasal strep infection results in activation of a pro-inflammatory type of T cell, known as Th17 cells, and opening of the BBB Inflammation, infection, autoimmunity, and brain injuries increase BBB permeability, allowing inflammatory cytokines, chemokines, other blood-born proteins, and immune cells (peripheral macrophages, B and T cells, including CD4+ T cells) to enter the brain parenchyma and promote disease progression. BBB permeability is a well-recognized characteristic of PANDAS.

In spite of the rare incidence of CNS symptoms associated with GAS infection, including brain abscesses, meningitis, and PANDAS, GAS is known to induce autoantibody in PANDAS and has been reported to be associated with recurrent streptococcal infection, however some brain illnesses from GAS are associated with illness caused by recurrence without GAS in the brain. Further to these reports, a recent study indicates that subcutaneous GAS infection causes permeability of the BBB and promotes microglial activation in a mouse model of infection, thus supporting the notion that disruption of the BBB by strep infection could be an early and significant event in neuroinflammation.

The peptides of the invention activate the cholinergic anti-inflammatory response and thus peptide immunotherapy creates an interaction between the nervous system and the immune system that contributes to reduction in inflammation useful in the treatment of PANS and PANDAS. Activation of the alpha7 nicotinic acetylcholine receptor upregulates BBB function by increasing the activity of the tight junction proteins known as claudin-5 and occluding expression in brain endothelium underscoring the therapeutic advantage for the use of the peptides disclosed herein in treating diseases that are caused by, or accelerated by, BBB permeability, including PANDAS, AD, and Parkinson's Disease.

The α7nAChR agonists are useful promoting wound healing of a chronic wound. The wound can be a surgical wound or a burn. In some embodiments, the wound can be a chronic wound such as an ulcer. In some embodiments, the method further comprises delivering a wound medication to the subject, the wound medication comprising one or more of: a cytotoxic drug, an antibiotic, an antiseptic, nicotine, an anti-platelet drug, an NSAID, colchicine, an anti-coagulant, a vasoconstricting drug or an immunosuppressive, a growth factor, an antibody, a protease, a protease inhibitor, an antibacterial peptide, an adhesive peptide, a hemostatic agent, living cells, honey, or nitric oxide.

The α7nAChR agonists are also useful for treating or improving cognition or cessation of addictions such as smoking or vaping, alcohol and/or drugs in a subject in need thereof.

The α7nAChR agonists are useful for treating a proliferative and non-neuronal immune disorders. A non-neuronal immune disorder, as used herein is an immune disorder that is associated with a cell or tissue other than a neuron. Non-neuronal immune disorders include but are not limited to disorders such as autoimmune disease, Inflammatory Bowel Disease, Crohn's disease, asthma, macular degeneration (e.g., dry AMD, wet AMD), retinopathy (e.g., diabetic retinopathy), kidney disease, preeclampsia, type 1 diabetes, hypertension, arthritis (e.g., osteoarthritis, rheumatoid arthritis (RA), psoriatic arthritis and sepsis.

Asthma as used herein refers to an allergic disorder of the respiratory system characterized by inflammation and narrowing of the airways, and increased reactivity of the airways to inhaled agents. Symptoms of asthma include recurrent episodes of wheezing, breathlessness, chest tightness, and coughing, resulting from airflow obstruction. Asthma likely results from complex interactions among inflammatory cells, mediators, and other cells and tissues resident in the airways. Mast cells, eosinophils, epithelial cells, macrophage, and activated T cells all play an important role in the inflammatory process associated with asthma.

The activity of the therapeutic peptides used in accordance with the present invention can be determined by any method known in the art or shown in the examples. In one embodiment, the activity of a therapeutic peptide is determined by using various experimental animal models, including but not limited to, cancer animal models such as scid mouse model or nude mice with human tumor grafts.

Various in vitro and in vivo assays that test the activities of a therapeutic peptide are used in purification processes of a therapeutic peptide. The protocols and compositions of the invention are also preferably tested in vitro, and then in vivo, for the desired therapeutic or prophylactic activity, prior to use in humans.

For instance, the therapeutic peptide binds to alpha 7 nAChR, preferably in a selective manner. As used herein, the terms “selective binding” and “specific binding” are used interchangeably to refer to the ability of the peptide to bind with greater affinity to alpha 7 nAChR and fragments thereof than to unrelated proteins.

Peptides can be tested for their ability to bind to alpha 7 nAChR using standard binding assays known in the art or the assays experimental and computational described in the examples. As an example of a suitable assay, alpha 7 nAChR can be immobilized on a surface (such as in a well of a multi-well plate) and then contacted with a labeled peptide. The amount of peptide that binds to the alpha 7 nAChR (and thus becomes itself immobilized onto the surface) may then be quantitated to determine whether a particular peptide binds to alpha 7 nAChR. Alternatively, the amount of peptide not bound to the surface may also be measured. In a variation of this assay, the peptide can be tested for its ability to bind directly to an alpha 7 nAChR-expressing cell.

Compounds for use in therapy can be tested in suitable animal model systems prior to testing in humans, including but not limited to in rats, mice, chicken, cows, monkeys, rabbits, etc.

The therapeutic peptides bind to alpha 7 nAChR, preferably in a selective manner. As used herein, the terms “selective binding” and “specific binding” are used interchangeably to refer to the ability of the peptide to bind with greater affinity to alpha 7AChR and fragments thereof than to other compounds. That is, peptides that bind selectively to alpha 7 nAChR will not bind to other compounds to the same extent and with the same affinity as they bind to alpha 7 nAChR and fragments thereof. A peptide that binds selectively to alpha 7 nAChR and to MHCII antigen binding groove will not bind to other compounds to the same extent and with the same affinity as they bind to those components.

The invention also encompasses small molecules that bind to alpha 7 nAChR. Such binding molecules may be identified by conventional screening methods, such as phage display procedures (e.g. methods described in Hart et al., J. Biol. Chem. 269:12468 (1994)). Hart et al. report a filamentous phage display library for identifying novel peptide ligands. In general, phage display libraries using, e.g., M13 or fd phage, are prepared using conventional procedures such as those described in the foregoing reference. The libraries generally display inserts containing from 4 to 80 amino acid residues. The inserts optionally represent a completely degenerate or biased array of peptides. Ligands having the appropriate binding properties are obtained by selecting those phage which express on their surface a ligand that binds to the target molecule. These phage are then subjected to several cycles of reselection to identify the peptide ligand expressing phage that have the most useful binding characteristics. Typically, phage that exhibit the best binding characteristics (e.g., highest affinity) are further characterized by nucleic acid analysis to identify the particular amino acid sequences of the peptide expressed on the phage surface in the optimum length of the express peptide to achieve optimum binding. Phage-display peptide or antibody library is also described in Brissette R et al Curr Opin Drug Discov Devel. 2006 May; 9(3):363-9.

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment. Multiple doses of the molecules of the invention are also contemplated.

The therapeutic peptides described herein can be used alone or in conjugates with other molecules such as detection or cytotoxic agents in the detection and treatment methods of the invention, as described in more detail herein.

Typically, one of the components usually comprises, or is coupled or conjugated to a detectable label. A detectable label is a moiety, the presence of which can be ascertained directly or indirectly. Generally, detection of the label involves an emission of energy by the label. The label can be detected directly by its ability to emit and/or absorb photons or other atomic particles of a particular wavelength (e.g., radioactivity, luminescence, optical or electron density, etc.). A label can be detected indirectly by its ability to bind, recruit and, in some cases, cleave another moiety which itself may emit or absorb light of a particular wavelength (e.g., epitope tag such as the FLAG epitope, enzyme tag such as horseradish peroxidase, etc.). An example of indirect detection is the use of a first enzyme label which cleaves a substrate into visible products. The label may be of a chemical, peptide or nucleic acid molecule nature although it is not so limited. Other detectable labels include radioactive isotopes such as P³² or H³, luminescent markers such as fluorochromes, optical or electron density markers, etc., or epitope tags such as the FLAG epitope or the HA epitope, biotin, avidin, and enzyme tags such as horseradish peroxidase, β-galactosidase, etc. The label may be bound to a peptide during or following its synthesis. There are many different labels and methods of labeling known to those of ordinary skill in the art. Examples of the types of labels that can be used in the present invention include enzymes, radioisotopes, fluorescent compounds, colloidal metals, chemiluminescent compounds, and bioluminescent compounds. Those of ordinary skill in the art will know of other suitable labels for the peptides described herein, or will be able to ascertain such, using routine experimentation. Furthermore, the coupling or conjugation of these labels to the peptides of the invention can be performed using standard techniques common to those of ordinary skill in the art.

Another labeling technique which may result in greater sensitivity consists of coupling the molecules described herein to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

Conjugation of the peptides to a detectable label facilitates, among other things, the use of such agents in diagnostic assays. Another category of detectable labels includes diagnostic and imaging labels (generally referred to as in vivo detectable labels) such as for example magnetic resonance imaging (MRI): Gd(DOTA); for nuclear medicine: ²⁰¹Tl, gamma-emitting radionuclide 99mTc; for positron-emission tomography (PET): positron-emitting isotopes, (18)F-fluorodeoxyglucose ((18)FDG), (18)F-fluoride, copper-64, gadodiamide, and radioisotopes of Pb(II) such as 203Pb; 111In.

The conjugations or modifications described herein employ routine chemistry, which chemistry does not form a part of the invention and which chemistry is well known to those skilled in the art of chemistry. The use of protecting groups and known linkers such as mono- and hetero-bifunctional linkers are well documented in the literature and will not be repeated here.

As used herein, “conjugated” means two entities stably bound to one another by any physiochemical means. It is important that the nature of the attachment is such that it does not impair substantially the effectiveness of either entity. Keeping these parameters in mind, any covalent or non-covalent linkage known to those of ordinary skill in the art may be employed. In some embodiments, covalent linkage is preferred. Noncovalent conjugation includes hydrophobic interactions, ionic interactions, high affinity interactions such as biotin-avidin and biotin-streptavidin complexation and other affinity interactions. Such means and methods of attachment are well known to those of ordinary skill in the art.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

The conjugates also include a peptide conjugated to another peptide such as CD4, gp120 or gp21. CD4, gp120 and gp21 peptides are all known in the art.

The active agents of the invention are administered to the subject in an effective amount for treating disorders such as autoimmune disease, Alzheimer's disease, graft rejection, and cancer. An “effective amount”, for instance, is an amount necessary or sufficient to realize a desired biologic effect. An “effective amount for autoimmune disease may be an amount sufficient to prevent or inhibit a decrease in T_{H} cells compared to the levels in the absence of peptide treatment. According to some aspects of the invention, an effective amount is that amount of a compound of the invention alone or in combination with another medicament, which when combined or co-administered or administered alone, results in a therapeutic response to the disease, either in the prevention or the treatment of the disease. The biological effect may be the amelioration and or absolute elimination of symptoms resulting from the disease. In another embodiment, the biological effect is the complete abrogation of the disease, as evidenced for example, by the absence of a symptom of the disease.

The effective amount of a compound of the invention in the treatment of a disease described herein may vary depending upon the specific compound used, the mode of delivery of the compound, and whether it is used alone or in combination. The effective amount for any particular application can also vary depending on such factors as the disease being treated, the particular compound being administered, the size of the subject, or the severity of the disease or condition. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

The agent may comprise different types of carriers depending on whether it is to be administered in solid, liquid or aerosol form, and whether it need to be sterile for such routes of administration as injection. The present invention can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences (1990), incorporated herein by reference). In a particular embodiment, intraperitoneal injection is contemplated.

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The composition of the invention can be used directly or can be mixed with suitable adjuvants and/or carriers. Suitable adjuvants include aluminum salt adjuvants, such as aluminum phosphate or aluminum hydroxide, calcium phosphate nanoparticles (BioSante Pharmaceuticals, Inc.), ZADAXIN™, nucleotides ppGpp and pppGpp, killed Bordetella pertussis or its components, Corenybacterium derived P40 component, cholera toxin and mycobacteria whole or parts, and ISCOMs (DeVries et al., 1988; Morein et al., 199&, Lovgren: al., 1991). Also useful as adjuvants are Pam3Cys, LPS, ds and ss RNA. The skilled artisan is familiar with carriers appropriate for pharmaceutical use or suitable for use in humans.

The composition of the invention can be administered in various ways and to different classes of recipients.

The compounds of the invention may be administered directly to a tissue. Direct tissue administration may be achieved by direct injection. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

According to the methods of the invention, the compound may be administered in a pharmaceutical composition. In general, a pharmaceutical composition comprises the compound of the invention and a pharmaceutically-acceptable carrier.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids, such as a syrup, an elixir or an emulsion.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

An alternative to producing therapeutic peptide or a fragment thereof by recombinant techniques is peptide synthesis. For example, an entire therapeutic peptide, or a peptide corresponding to a portion of therapeutic peptide can be synthesized by use of a peptide synthesizer. Conventional peptide synthesis or other synthetic protocols well known in the art may be used.

Peptides having the amino acid sequence of therapeutic peptide or a portion thereof may be synthesized by solid-phase peptide synthesis using procedures similar to those described by Merrifield, 1963, J. Am. Chem. Soc., 85: 2149. During synthesis, N-α-protected amino acids having protected side chains are added stepwise to a growing polypeptide chain linked by its C-terminal and to an insoluble polymeric support, i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting therapeutic peptide or a fragment thereof is accomplished using conventional procedures, such as preparative HPLC using gel permeation, partition and/or ion exchange chromatography. The choice of appropriate matrices and buffers are well known in the art and so are not described in detail herein.

The invention also includes articles, which refers to any one or collection of components. In some embodiments the articles are kits. The articles include pharmaceutical or diagnostic grade compounds of the invention in one or more containers. The article may include instructions or labels promoting or describing the use of the compounds of the invention.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment of disease.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are sued, the liquid form may be concentrated or ready to use. The solvent will depend on the compound and the mode of use or administration. Suitable solvents for drug compositions are well known and are available in the literature. The solvent will depend on the compound and the mode of use or administration.

The kits, in one set of embodiments, may comprise a carrier means being compartmentalized to receive in close confinement one or more container means such as vials, tubes, and the like, each of the container means comprising one of the separate elements to be used in the method. For example, one of the containers may comprise a positive control for an assay. Additionally, the kit may include containers for other components, for example, buffers useful in the assay.

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery.

In a preferred embodiment, the unit dosage form is suitable for intravenous, intramuscular or subcutaneous delivery. Thus, the invention encompasses solutions, preferably sterile, suitable for each delivery route.

In another preferred embodiment, compositions of the invention are stored in containers with biocompatible detergents, including but not limited to, lecithin, taurocholic acid, and cholesterol; or with other proteins, including but not limited to, gamma globulins and serum albumins. More preferably, compositions of the invention are stored with human serum albumins for human uses, and stored with bovine serum albumins for veterinary uses.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures (such as methods for monitoring mean absolute lymphocyte counts, tumor cell counts, and tumor size) and other monitoring information.

More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material. The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising a needle or syringe, preferably packaged in sterile form, for injection of the formulation, and/or a packaged alcohol pad.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1: Alpha7 nAChR Anti-Inflammatory Mechanism of Peptide Agonist

INTRODUCTION: A study was conducted to evaluate a potential alpha7 nAChR anti-inflammatory mechanism of the MKR-4 peptide, using the alpha7 neuronal nicotinic acetylcholine receptor (alpha 7 nAChR) antagonist methyllycaconitine (MLA).

SUMMARY: A study was conducted to evaluate a potential alpha7 nAChR anti-inflammatory mechanism of the MKR-4 peptide, using the alpha7 nAChR antagonist methyllycaconitine (MLA). After enrollment, animals in Groups 2-4 were sensitized ID at the base of the tail with 0.1 mL FCA on Day 1. On Days 2, 3, 5 & 7 Groups were then dosed intraperitoneally (IP) with either: peptide and MLA in combination (2 mg/kg and 6 mg/kg, respectively); peptide (2 mg/kg) and MLA vehicle (saline) in combination; or peptide vehicle (saline) and MLA vehicle (saline). Animals were dosed once on Study Day 2 (MLA or MLA vehicle), and Days 3, 5 & 7 (Peptide or Peptide vehicle, MLA or MLA vehicle) as described in Table 2 below. Body weights were recorded on Study Days 0 & 8. Mice were euthanized for necropsy on Study Day 8, and serum and whole spleens were collected from all animals and sent to the sponsor for internal FACS analysis. Toxicity and Peptide mechanistic evaluation was based on absolute body weight change, spleen weights and sponsor FACS analysis (not included in report). All mice survived to study termination. Though none of the group body weight change differences or spleen weight differences were significant, the data suggested that the Peptide might be antagonizing a body weight change decrease and spleen weight increase induced by CFA by stimulating the alpha7 nAChR, and that the MLA could be antagonizing a Peptide tendency to decrease spleen size via blocking the alpha? nAChR.

Materials and Methods:

Boulder BioPath, Boulder, Colo. Mice were housed in the Bolder BioPath animal facility according to AALAC and IACUC regulations.

Cell Culture. Mice were sacrificed and spleens were removed. The tissues were dissociated, from which single cell suspensions were made by passing the spleens through 40 μm cell strainers. Red blood cells were lysed using ACK Buffer (Sigma, Inc). Cells were then cultured with TLR ligands at the designated concentrations at 1.010⁶ cells/mL in 6 well plates. Cells were grown in RPMI 1640 (Invitrogen) supplemented with 5% fetal bovine serum (Invitrogen) in a humidified 5% CO2 incubator at 37° C. for the designated time period.

Isolation of Splenic Cells. Splenocytes were isolated from 8 week old C57BL/6J mice. Following sacrifice, spleens were removed and splenocytes isolated as above. Cells were then resuspended in complete RPMI and were cultured with the TLR ligands at the designated concentrations at 10⁶ cells/mL in 6 well plates. Cells were grown in RPMI 1640 (Invitrogen) supplemented with 5% fetal bovine serum (Invitrogen) in a humidified 5% CO2 incubator at 37° C. for the designated time period.

Toll Like Receptor Ligands. The following TLR ligand was used at 5 μg/mL) CpG-ODN 2006 (Invivogen), 5′-tcgtcgttttgtcgttttgtcgtt-3′ (24 mer); bases are phosphorothioate (nuclease resistant).

Antibodies and Flow Cytometry. Single cell suspensions were made of tissues or cells harvested from culture and stained with the following monoclonal antibodies: a monoclonal antibody directed against mouse alpha 7 nicotinic acetylcholine receptor (alpha 7 nAChR) (Santa Cruz Biotechnology); anti-mouse MHC Class II (M5114), and anti-mouse CLIP 15G4, Santa Cruz. Following staining, cells were analyzed comparing each level of staining to the appropriate isotype control on a Beckman Coulter Cytoflex benchtop flow cytometer. Data was analyzed using FlowJo software (Tree Star Inc.).

Experimental Design: On Study Day 0, mice were weighed and randomized by body weight into treatment groups. Following randomization, on Study Day 1, Groups 2-4 were sensitized ID at the base of the tail with 0.1 mL of FCA. Drug and vehicle dosing was administered as indicated in the Table below. When MLA and Peptide (FRIMAVLAS (SEQ ID NO: 389))—or their vehicles—were dosed on the same day, the MLA antagonist or vehicle was dosed 1 hour before the Peptide or its vehicle.

| | | # | | Dose | Dosing |
|---|---|---|---|---|---|
| Group | Treatments | Mice | Dose | Route | Days |
| Group 1 | Veh (peptide) | 5 | NA | IP/IP | 3, 5, 7/2, |
| | Veh (MLA) | | | | 3, 5, 7 |
| Group 2 | CFA Veh | 5 | NA | ID/IP/IP | 1/3, 5, 7/2, |
| | (peptide) | | | | 3, 5, 7 |
| | Veh (MLA) | | | | |
| Group 3 | CFA/Peptide | 5 | 2 mg/Kg | ID/IP/IP | 1/3, 5, 7/2, |
| | MLA (Veh) | | | | 3, 5, 7 |
| Group 4 | CFA/Peptide/ | 5 | 2 mg/Kg | ID/IP/IP | 1/3, 5, 7/2, |
| | MLA | | 6 mg/Kg | | 3, 5, 7 |

On Study Day 8, mice were anesthetized with Isoflurane (VetOne, Cat #502017) and bled to exsanguination followed by cervical dislocation for necropsy and tissue collection.

Test Article Formulation Instructions & Calculations: Peptide dosing solution (0.2 mg/mL) was made fresh daily within 1 hour of use by diluting 0.09 mL of 5 mg/mL DMSO stock into 2.11 mL sterile saline. DMSO stock was kept at 4 C, then refrozen (to avoid repeated freeze-thaw cycles). Still, stock was relatively “solid” even at 4 C.

MLA antagonist dosing solution (0.6 mg/mL) was made on Day 2 by diluting the entire 5 mg in the sealed vial with 8.3 mL of sterile saline and aliquoting out ˜1.1 mL in each of seven 2 mL cryovials and freezing at −20 C. Four vials were used in the experiment, each thawed within 1 hour of use. Three vials remain frozen.

Dosing Formulations and Vehicle Storage & Stability: Saline vehicle is stable at 4 C for months. Peptide dosing solutions were made up and used fresh daily. DMSO stock was stable for a few months. MLA frozen “stock” solutions were stable for up to 1 month.

Statistical Analysis: Data for each animal were entered into Microsoft Excel and means and standard errors for each group were calculated. Test groups were compared to vehicle controls using a one-way analysis of variance (1-way ANOVA) with a Dunnett's post-hoc analysis for measured (parametric) data. ANOVA analysis was performed using Prism 8.0d software (GraphPad). Unless indicated, Bolder BioPATH, Inc. performs statistical analysis on raw (untransformed) data only. Statistical tests make certain assumptions regarding the data's normality and homogeneity of variance, and further analysis may be required if testing resulted in violations of these assumptions. Significance for all tests was set at p<0.05, and p values were rounded to the third decimal place.

Results:

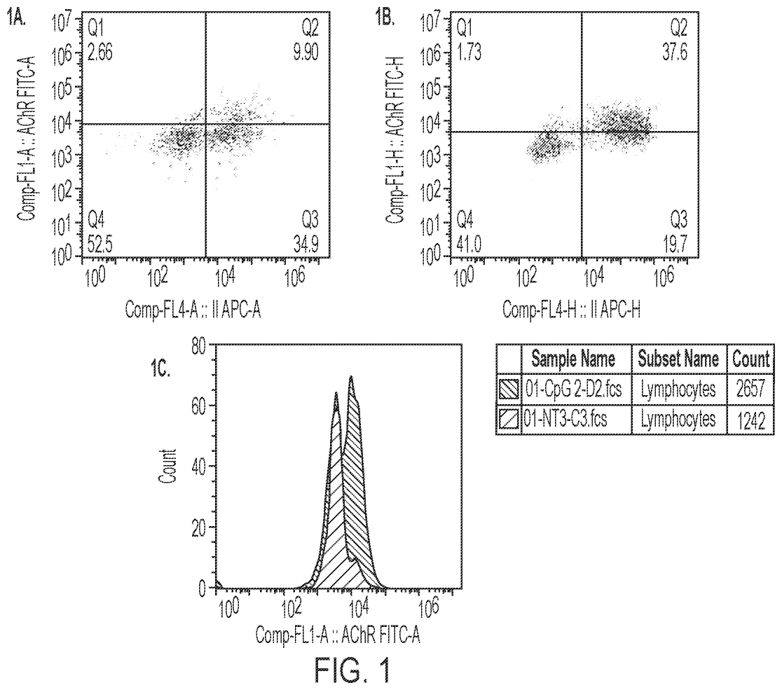

The data is presented in FIGS. 1A-1C. It was demonstrated that activation of TLR 9 results in increased alpha 7 nicotinic acetylcholine receptor expression on B cells. In FIG. 1A the X-axis represents fluorescence intensity of MHC class II expression on untreated splenic B cells from a C57BL6 mouse. The Y-axis represents the level of expression of alpha 7 nicotinic acetylcholine receptor (α7nnAChR) on untreated splenic B cells (as indicated by MHC class II expression). The data indicate that 9% of the total spleen cells are B cells that express the alpha 7 nAChR. As shown in FIG. 1B treatment with the TLR 9 agonist CpG resulted in increased numbers of B cells expressing alpha 7 nAChR. The data indicate that a total of 37.6% of the total splenocytes express alpha 7 nAChR after CpG treatment. FIG. 1C is a graph in which the Y axis in this histogram represents the cell numbers. The X axis reflects the relative fluorescence intensity before (green histogram) and after treatment with the TLR 9 agonist CpG (blue histogram). These data show that the relative level of expression of nAChR per cell increases as a result of CpG treatment.

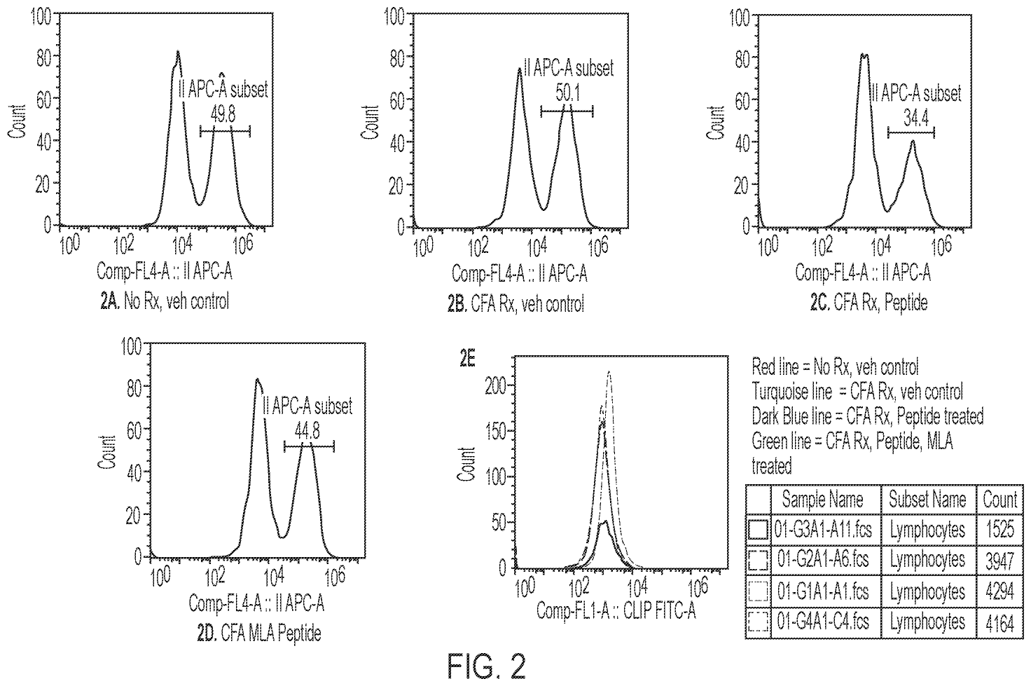

The data demonstrates that the peptide is functioning through the alpha 7 nACh pathway. The effects of a competitive antagonist peptide can be reversed by Methyllylaconitine (MLA), a specific inhibitor of alpha 7 nAChR activation as shown in FIGS. 2A-2E. Animals were randomized into groups of vehicle treated (2A), Complete Freund's Adjuvant (CFA) treated 2B), CFA treated and Peptide treated (2C), or CFA treated, Peptide treated, and treated with MLA (2D). The percentage of splenic B cells in each group is indicated by the region gates: 2A, 49.8%; 2B, 50.1%; 2C, 34.4%; and 2D, 44.8%. The results show that the effects of peptide treatment of CFA treated animals can be reversed with MLA. FIG. 2E shows the Peptide induced reduction in CLIP+ B cells is reversed by treatment with MLA. The Y axis in this histogram represents the cell numbers. The X axis reflects the relative fluorescence intensity of CLIP on B cells with the decreased number of CLIP+ B cells reversed by treatment with MLA, suggesting the peptide dependent reduction in CLIP+ B cells is alpha 7 nAChR mediated.

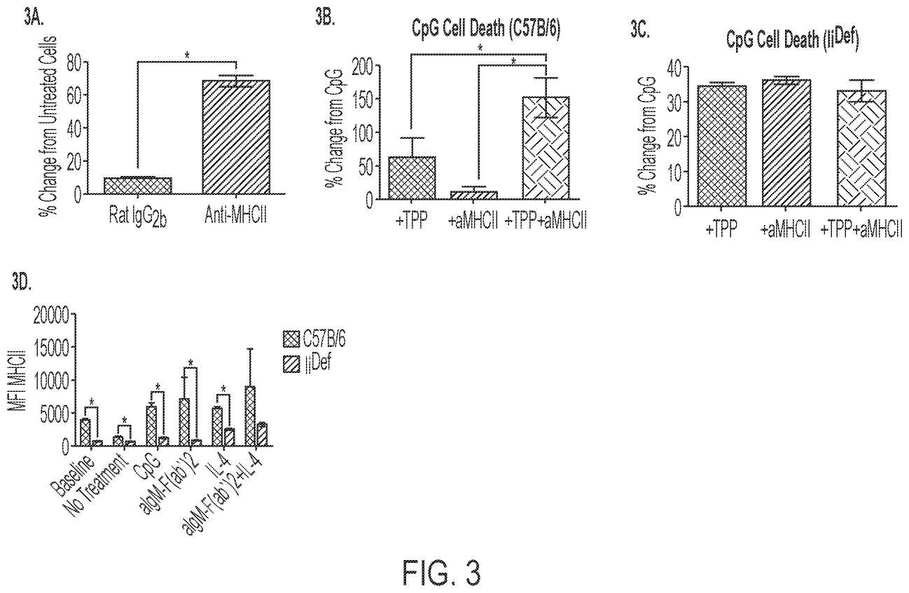

The data also demonstrates that CpG-mediated CLIP expression protects B cells from MHCII mediated cell death. FIG. 3A shows the % change in cell death of resting, C57B/6 B cells treated with anti-MHCII (M5/114) or the isotype control rat IgG2b. FIG. 3B shows that change in cell death of CpG activated C57Bl6 B cells treated with peptide (TPP), anti-MHCII (M5114), or peptide followed by anti-MHCII (M5114). FIG. 3C shows the % change in cell death of CpG activated Invariant Chain deficient (Ii) C57Bl6 B cells treated with peptide (TPP), anti-MHCII (M5114), or peptide followed by anti-MHCII (M5114). FIG. 3D shows the mean fluorescence intensity (MFI) of MHCII on B cell activated as labeled for 48 hours. C57B/6 (solid black bars), IiDef (solid grey bars). * designates a p value <0.05 compared to the selected group.

### Example 2: Permeability of the Blood Brain Barrier Results from Intranasal Streptoccocal Infection

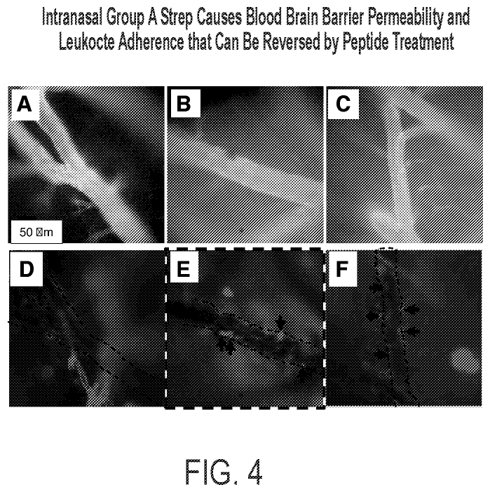

Whether intranasal infection with Streptococcus pyogenes would induce blood-brain barrier permeability in C57 BL/6J mice by a TLR-dependent mechanism was examined. Immediately following craniotomy, mice were placed under an intravital microscope to study vascular permeability and leukocytes. Permeability of cerebral blood vessels was observed using FITC-dextran fluorescent dye and, tracing of leukocytes is observed by labeling such cells with Rhodamine-6G. Intranasal infection with Streptococcus pyogenes (gram positive) increased BBB permeability (FIG. 4) and the number of adherent or rolling lymphocytes (FIG. 4) within 24 hours post-infection, compared to saline treated controls. Interestingly, similar results were observed in Pam-3-Cys (P3C) treated mice at 24 hours post-treatment (i.p. injected). Together, the data suggest that TLR 1/2 activation is sufficient to induce BBB permeability and increase adherence of lymphocytes to microvasculature that comprise the BBB. Furthermore, the data suggest that TLR-driven BBB permeability, polyclonal expansion of lymphocytes and adherence of such cells, acutely following infection, could facilitate migration of such cells and antibodies into the brain. Of great importance to the fields of MS, ALS, PANS/PANDAS research and other autoimmune neuropsychiatric disorders, the data show for the first time that TLR 1/2 activation, alone, induces BBB permeability, suggesting a possible mechanism for humoral and cellular autoimmunity in these and other post-infectious neuropsychiatric disorders.

Methods: To test the hypothesis that intranasal infection may increase BBB permeability, we exposed mice to GAS via intranasal infection with Streptococcus pyogenes (GAS). We assessed BBB permeability using intravital microscopy, and we characterized the immune response to GAS using flow cytometric analysis of nasal associated lymphoid tissue (NALT), spleen, and brain.

Animal Use and Welfare Disclaimer

Bacterial culture and preparation for intranasal infection: Group A Streptococcus pyogenes (obtained from Dr. P. Patrick Cleary of University of Minnesota) were grown on sheep blood agar and in Todd-Hewitt broth supplemented with 2% Neopeptone. To prepare bacteria for intranasal infection, 2-3 colonies were taken from blood agar plates and inoculated in 10 mL of THB-N, at 37C overnight. Following day, overnight culture was diluted 1:10 in additional THB-N and OD₆₀₀ of diluted overnight culture was assessed for logarithmic growth, OD₆₀₀=0.5-0.6; roughly 3-4 hours after dilution. Bacterial suspension was then centrifuged for 10 minutes at 4000 RPM at room temperature. Supernatant was removed, and bacterial pellet was resuspended in 10 mL of PBS at room temperature (RT). Resuspended bacteria were transferred to 1.5 mL Eppendorf tubes and centrifuged for 1 minute at full speed in microfuge (12000 RPM). Supernatant was removed, cells carefully washed in 1 mL of PBS (RT), without disturbing pellet, and centrifuged again at maximum speed for 1 minute. Lastly, supernatant was removed and pellet resuspended in 150 uL of PBS (at RT). To properly dose bacteria, OD₆₀₀ was reassessed in bacterial suspensions and diluted as needed to reach OD₆₀₀=0.5-0.6, a dose of 2×10⁸ CFU/mouse (15 uL per mouse, 7.5 uL per nares, see below).

Intranasal infection: Isoflurane gas was employed to briefly anesthetize mice (1-2 minutes). Once anesthetized, 7.5 uL of streptococcal bacterial suspension (OD₆₀₀=0.5-0.6) was placed on each nares (outer nostril) of mouse. Control mice received intranasal inoculation with vehicle (PBS). The droplets were inhaled into nasal sinuses. This small volume avoids inoculation of the lungs, but reproducibly results in colonization of nasal-associated lymphoid tissue (NALT, Park 2003; Wang 2010).

Intraperitoneal injection: The TLR 1/2 agonist, Pam-3-Cys (25 ug/mouse; Pam3CSK4, InvivoGen, San Diego Calif.) was intraperitoneally (right side) injected into mice 24 hours before intravital microscopy. P3C causes immune cell activation and polycolonal expansion of B-lymphocytes by Toll-Like receptor ligation (Newell 2010). Control mice received equal volume of vehicle (PBS) intraperitoneally.

MHC class II-targeted proprietary peptide (TPP, Elim Biopharmaceuticals Inc., Hayward Calif.; also see Newell 2010) was administered intraperitoneally (5 ug/mouse, left side), sometimes in conjunction with P3C (right side). TPP was dissolved in DMSO for a final concentration of 2.5% DMSO in PBS. Control mice received vehicle (2.5% DMSO in PBS) intraperitoneally.

Preparation of Mice: Mice were anesthetized with an (i.p.) injection of 50% urethane (Sigma-Aldrich, U2500) in PBS (4 mg/Kg) and maintained on heating pad. Anesthetic plane was assessed throughout preparation and experiments, by response to toe pinch. If needed, urethane supplement was given at 10% of original dose volume. To prepare mice tails for intravenous (i.v.) dye injection we maintained anesthetized mice on a heating pad for 10-15 minutes, and then briskly wiped tails (toward tail-end) with 70% ethanol wipes to remove scales/hair and increase vasodilation. Immediately following tail wiping, Fluorescein dextran 10 kDa (10 mg/mL, Sigma-Aldrich, FD10S) was administered (2 uL/g mouse) via right lateral tail vein and, Rhodamine 6G (0.2 mg/mL, Sigma-Aldrich, 83697) was administered (1.6 uL/g mouse) via left lateral tail vein. When needed, tail was wiped multiple times to ensure clear visualization of tail veins. Following successful (i.v.) injection of dyes, mice underwent craniotomy.

Craniotomy: A craniotomy (removal of a small area of skull) was performed to expose the cerebrum and allow imaging of cerebral microvasculature (specifically pial vessels of pia mater). First, an incision across the midline of the scalp was made to expose the right parietal bone. The skull was then carefully marked with a waterproof marker, designating the area, 1 mm posterior from the Bregma and 4 mm lateral from the midline, to be removed (craniotomy diameter: 2.5 mm). Next, the craniotomy was performed with a drill ensuring the dura mater was not cut. Mice were excluded if researchers cut, perforated and caused severe hemorrhaging of dura mater, as this caused extensive bleeding and leaching of previously injected fluorescent dyes. A bolus of 0.9% saline was placed over the craniotomy and the animal was then placed under an intravital microscope for visualization of brain microvasculature.

Intravital Microscopy: Immediately following craniotomy, mice are placed under an intravital microscope to study vascular permeability and leukocytes. Permeability of cerebral blood vessels is observed using FITC-dextran fluorescent dye and, tracing of leukocytes is observed by labeling such cells with Rhodamine-6G. Blood pressure is monitored throughout microscopy (as stated above). Images and video for data analysis will be taken every 10 minutes for a total of 2 hours. After data collection is complete, all animals are euthanized.

Imaging: Images are taken via intravital microscopy for 1-3 hours hour every 20 minutes. At the end the animals that have received the Evans blue dye, the left ventricle is perfused with normal saline until clear fluid appears in the right arterial incision. Brain tissue is harvested for assays.

Results: Intranasal infection with GAS caused BBB permeability as measured by intravital microscopy. In vitro exposure of rat brain microvascular endothelial cells to the TLR agonist lipotekoic acid (LTA, a mimetic of GAS products) and/or polyinosinic polycytosinic acid (PIC) showed that LTA induced permeability, whereas PIC or PIC with LTA did not. Intranasal infection of mice with GAS caused a decrease in activated splenic lymphocytes, accompanied by a rapid expansion of B cells and T cells in the nasal-associated lymphoid tissue (NALT).

Streptococcus pyogenes, a gram-positive bacterium is implicated in post-infectious neuropsychiatric disorders, such as PANDAS. The data presented herein demonstrates the effects of gram-positive bacteria and their TLR products, such as P3C (a TLR 1/2 agonist), on the BBB. To determine if streptococcal infection or P3C alone can increase BBB permeability, mice were intranasally infected with Streptococcus pyogenes, or intraperitoneally injected P3C and assessed, 24 hours post-treatment, BBB permeability and the number of adhering or rolling lymphocytes within pial venules of the brain, in vivo. Prior to intravital microscopy, all mice were anesthetized, cranieotimized and received a bolus of fluorescein dextran (FITC-dextran, mw 3 KDa) in the left tail vein and, a bolus of Rhodamine 6G in the right tail vein, then placed under the intravital microscope. FITC-dextran was used to label whole blood, i.e. the fluid components of blood, for assessment of fluid extravasation from pial venules (REF) comprising the BBB, 24 hours post-treatment. Rhodamine 6G was used to generally label all leukocytes (lymphocytes) present in the blood (REF), for assessment of changes in lymphocyte behavior, i.e. rolling and adhering to pial venule walls.

Intranasal infection induces BBB permeability of and lymphocyte rolling and adhering to pial venules, in vivo.

Intranasal streptococcal infection significantly increased (p<0.05) BBB permeability at 24 hours post-treatment, compared to intranasal saline treated controls (FIGS. 4A, 4B; 5A). Furthermore, intranasal streptococcal infection significantly increased the number of adherent (p<0.001) or rolling (p<0.01) lymphocytes at 24 hours, compared to control (FIGS. 4D, 4E; 5B, 5C). We qualitatively determined the diameter of adherent lymphocytes using the Nikon imaging software used to capture intravital images/video. Streptococcal infection markedly increased the average diameter (FIG. 2D) of adherent lymphocytes (10 um; largest observed diameters were 15 um) compared to control (0 um, as zero adherent cells were observed in these control mice). These data suggest that streptococcal infections can acutely (within 24 hours) induce BBB permeability and changes in lymphocyte behavior.

Pam-3-Cys is sufficient to induce permeability of and lymphocyte rolling and adhering to pial venules, in vivo.

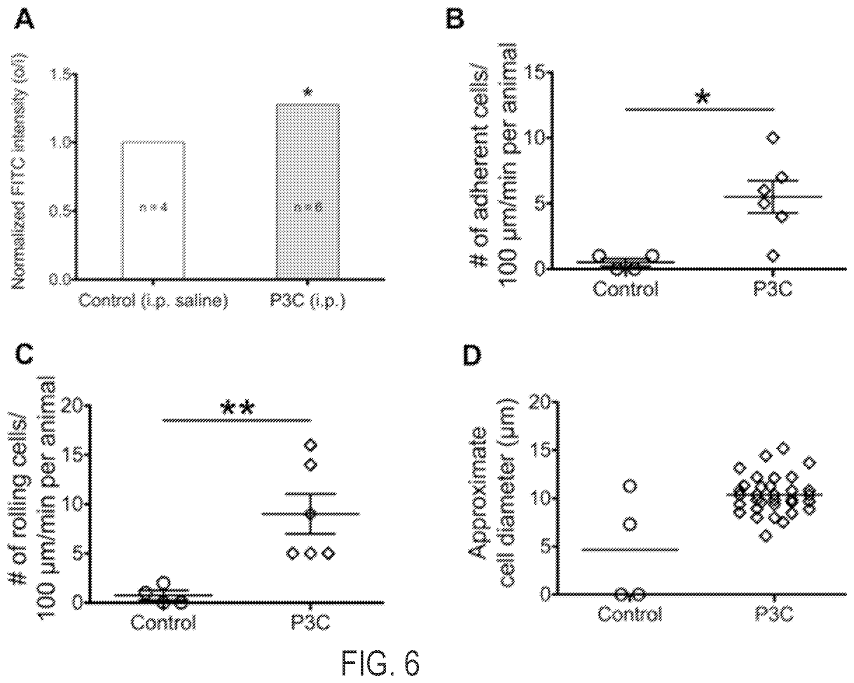

Because Streptococcus bacteria are implicated in many autoimmune disorders and bacterial products, such as TLRs can stimulate immune cells, we determined if a gram-positive derived TLR (P3C) could similarly affect BBB permeability and lymphocyte adhering and rolling, as did intranasal streptococcal infection. Interestingly, intraperitoneal (i.p) P3C injection, alone, significantly increased (p<0.05) BBB permeability at 24 hours post-treatment, compared to i.p. saline treated control (FIGS. 4G, 4H; 6A). P3C also significantly increased the number of adherent (p<0.05) or rolling (p<0.01) lymphocytes at 24 hours, compared to control (FIGS. 4J, 4K; 6B, 6C) Like streptococcal infection, P3C markedly increased the average diameter (FIG. 6D) of adherent lymphocytes (10 um; largest observed were 15 um) compared to control (5 um). These data suggest that TLR agonists from gram-positive bacteria are sufficient to induce BBB permeability and changes in lymphocyte behavior.

MHCII-CLIP dependent mechanism possibly drives BBB permeability.

Class II-associated invariant chain peptide (CLIP) is a cleavage product of CD74 invariant chain. CLIP is proposed to act as a placeholder in the groove of MHCII of antigen presenting cells (APC). TLR-activation of B cells results in ectopic CLIP expression and increased TNF-α release by T cells. To determine if increased BBB permeability and lymphocyte adhering and rolling can be attributed to TLR activated lymphocytes (i.e. CLIP+ APCs), we administered a competitive antagonist (TPP) to CLIP at the same time as intranasal infection or P3C treatment.

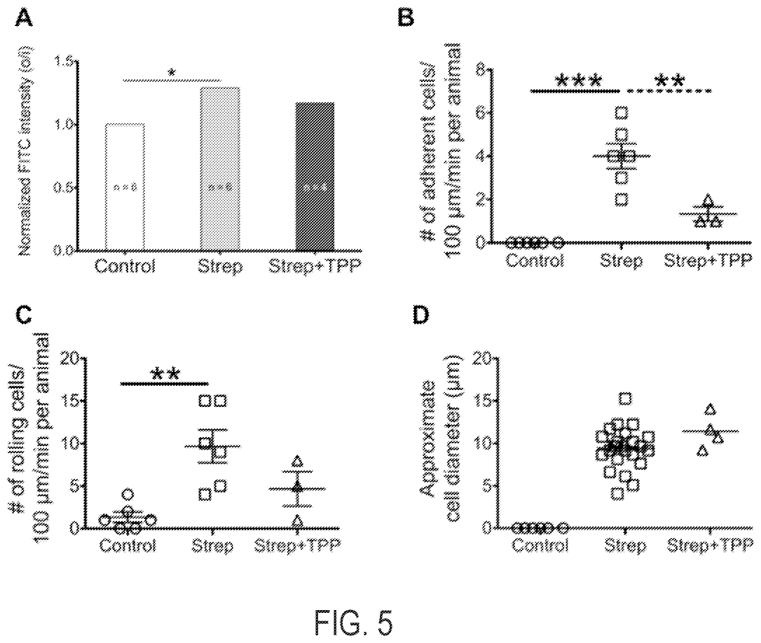

TPP treatment reversed streptococcal infection-induced BBB permeability (FIGS. 4C; 5A) and lymphocyte rolling and adhering (FIGS. 4F; 5B, 5C). Similarly, TPP treatment reduced P3C-induced BBB permeability (FIGS. 4I; 6A) and lymphocyte rolling and adhering (FIGS. 4L; 6B, 6C). However, TPP treatment did not attenuate the size (FIGS. 5D; 6D) of adherent cells in such treated mice (even though it reduced the number of adherent cells).

Conclusion: Here we demonstrate that intranasal infection with GAS results in a rapid local immune response and a rapid increase in BBB permeability. These results provide a mechanistic link between a bacterial infection and accompanying changes in the central nervous system that may be implicated in a variety of post-infectious neurological syndromes, including PANS.

### Example 3: Use of Peptides in Treatment of Traumatic Brain Injury (TBI)

It has been discovered that the immune molecule CD74, and its cleavage product, CLIP, contribute to TBI-induced neuropathology, and the data indicates that antagonizing CLIP can rescue post-traumatic neurobehavioral deficits.

Identification of inflammatory mediators in AD led to the hypothesis that neuroinflammation may be a key factor in the progression of neurodegeneration, and thousands of studies support neuroinflammation as a pathological hallmark of AD. TLR activation can trigger inflammation and amyloid β(A β) can bind to TLRs on microglial and other immune cells. Our data indicate that signaling via TLRs, such as TLR2, TLR4, and TLR6, induces expansion of pro-inflammatory cells that express Major Histocompatibility Complex (MHC) class II molecules, in which the peptide binding groove is filled with the breakdown product of CD74, known as MHC class II associated invariant peptide (CLIP). Displaying CLIP in MHC class II, prevents T cell recognition of the proinflammatory cell and promotes survival of proinflammatory cells. Importantly, aggregates of beta amylin bind to TLR and signal through the MyD88 complex, resulting in the expansion of pro-inflammatory cells that may be critically involved in A β-dependent neuroinflammation. It was unknown is whether Aβ binding to TLR on macrophages, microglia, or B cells in AD, causes an expansion of CLIP+ pro-inflammatory cells. To target TLR-mediated inflammatory responses, a competitive antagonist peptide (CAP) was designed, with a binding coefficient for MHC class II that is greater than that of CLIP in all known human and mouse MHC class II alleles.

Rodent models of AD and TBI: There are several commercially available AD mouse models that exhibit neurobehavioral and pathological symptomology, analogous to that seen in clinical cases. Some of these models have been incorporated into studies on TBI and inflammation. Initial experiments have used the 5×FAD mouse to assess TBI-induced innate and adaptive immune mechanisms of CD74, in MI/AD/ADRD. This animal model presents several advantages in this setting. Within months after birth, this animal model spontaneously develops quantifiable AD/ADRD-related neurobehavioral and neuropathological deficits, that are exacerbated by models of TBI. The animal model demonstrates intact antigen processing and presentation, quantifiable alterations to the adaptive immune response, and a build-up of amyloid-beta accumulation. Additionally, the mice are commercially available and there is adequate supply to enable experimental feasibility.

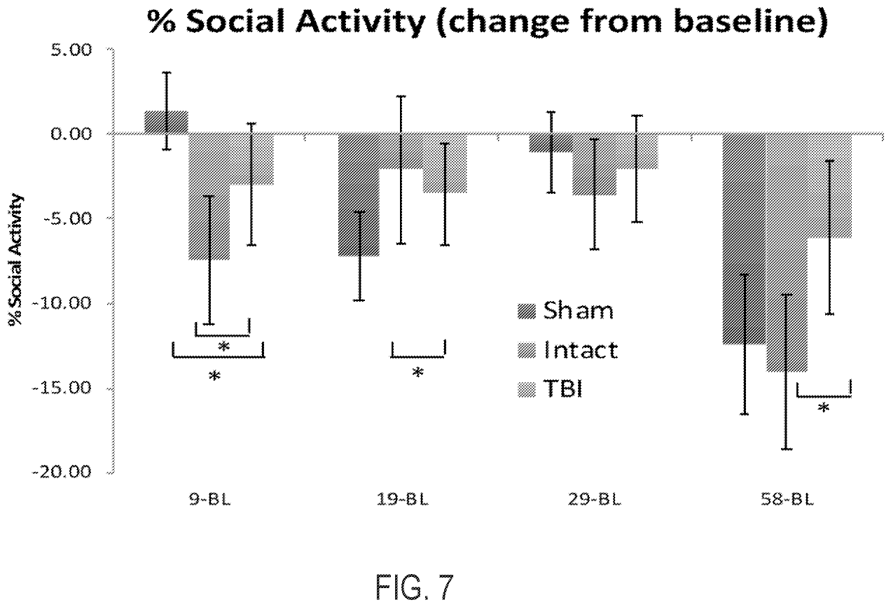

Cognitive and behavioral outcomes from 1-60 days after TBI and neuropathological outcomes at 1-90 days after TBI are being examined. Preliminary data shows that from 9-60 days after our model of TBI, rodents develop significantly impaired social interactions (FIG. 7), and a trend towards impairment on the sucrose preference test (not shown), compared to sham groups. The water maze task was removed, and replaced with a Barnes Maze task, for the purposes of the AD studies.

### Example 4: Competitive Antagonist Peptide (CAP) Treatment Suppresses Inflammatory Cytokines, Depletes Pro-Inflammatory B Cells, and Promotes Expansion of CD8+ T Cells

Here, data is presented in support of testing the hypothesis that competitive antagonist peptide (CAP) treatment suppresses inflammatory cytokines, depletes pro-inflammatory B cells, and promotes expansion of CD8+ T cells, at least in part via activation of the α7 nAChR.

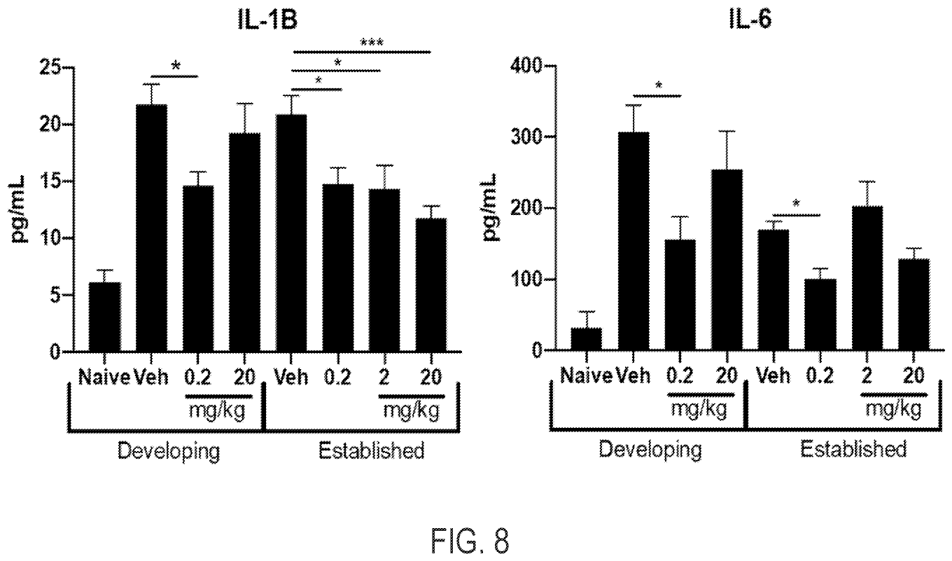

Central to the overarching hypothesis, is the anti-inflammatory effects of CAP. Data demonstrate that injecting a collagen emulsion in Complete Freunds Adjuvant (CFA) into mice, results in an increase in the pro-inflammatory cytokines, IL1β and IL6 (FIG. 8). This increase was observed in both the developing and established arthritis stages (FIG. 8). Treatment with 0.2 mg/kg of CAP (i.p.) during the developing stage was the most efficacious at reducing IL1β and IL6, whereas treatment with 20 mg/kg (i.p.) was most efficacious at reducing IL1β and IL6 during the established stage (FIG. 8).

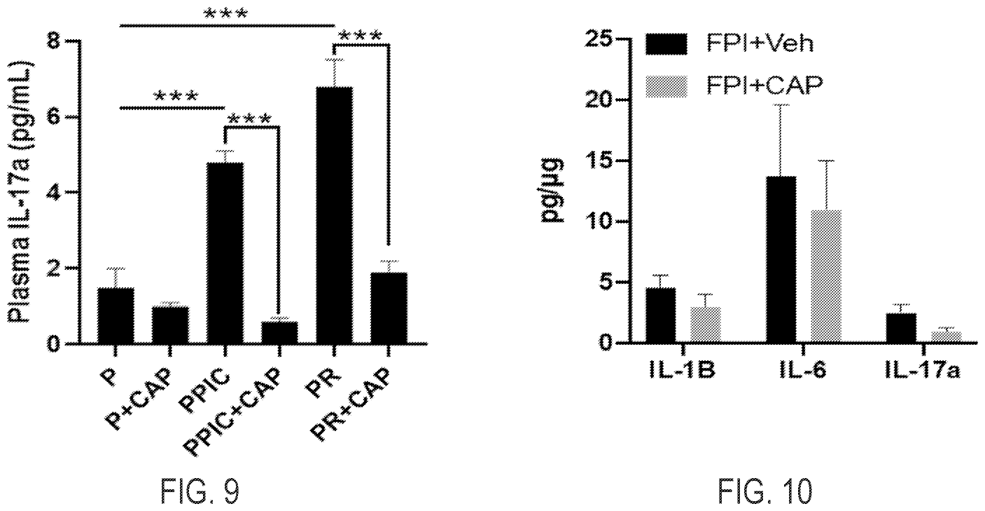

In a separate study, preeclampsia was induced in pregnant mice by injection of either the toll-like receptor 3 (TLR3) agonist, PolyI:C, or with the toll-like receptor 8 (TLR8) agonist R837. In both of these models of preeclampsia, IL17 was significantly elevated (FIG. 9) and this elevation was significantly reduced by i.p. CAP injection (FIG. 9). Whereas both of these studies were performed in the periphery, analysis of IL1b, IL6 and IL17, in the brains after TBI indicated that i.p. CAP treatment at 30 minutes after TBI, was able to reduce all 3 cytokines in the brain (FIG. 10). Although these results were not significant, there was a trend toward a reduction for all 3 cytokines, and subsequent power analysis indicated that the study in TBI mice was under-powered. It is pertinent to note that this power issue has been corrected.

Taken together, these 3 studies from distinct models that result in inflammation and neuroinflammation, clearly indicate the ability of CAP to significantly reduce key pro-inflammatory cytokines.

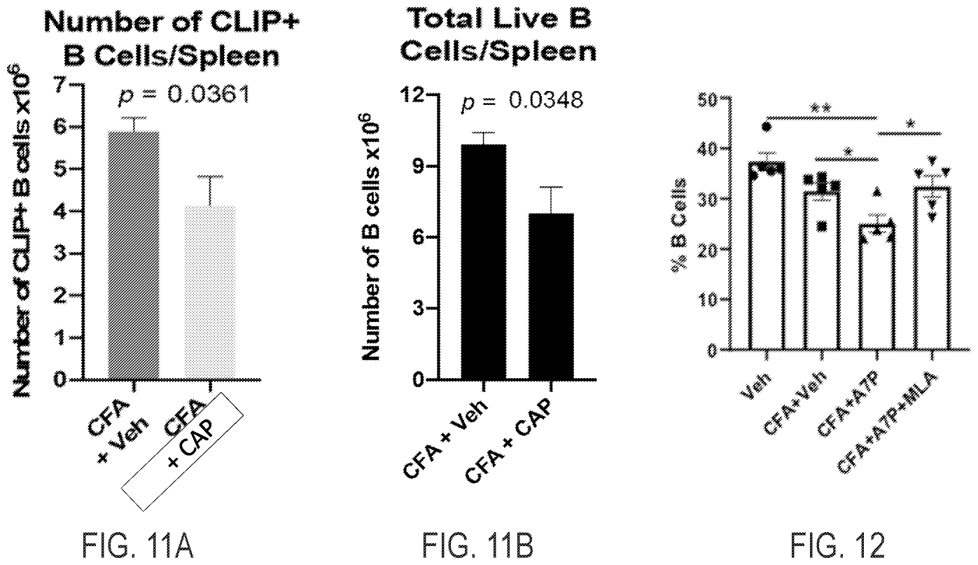

The next series of experiments are focused on the effects of CAP on splenic immune cells. Numerous published studies clearly indicate that one mechanism by which CAP works, is via its effects on B cell expansion and activation, and CLIP on B cells. Using the model of arthritis, in which CFA is injected into mice, it was first observed that CAP treatment beginning 3 days after CFA and then once every 3 days until day 10 after CFA (3 total CAP injections), significantly reduced the number of CLIP+ B cells (FIGS. 11A-11B). It was also observed that CFA treatment increased B cell expansion, and this expansion was reversed by the 3×CAP treatment (FIG. 12). As part of this experiment, the mice were co-treated with CAP and the alpha-7 nicotinic acetylcholine receptor (α7nAChR) antagonist, methyllycaconitine (MCA). Administering MCA with CAP prevented the CAP-induced decrease in B cell expansion, in the CFA model. Therefore, CAP is able to reduce B cell expansion and B cell expression of CLIP, and this action is blocked if the α7nAChR is blocked.

### Example 5: Targeting the Cholinergic Anti-inflammatory Pathway to Treat Viral Infection

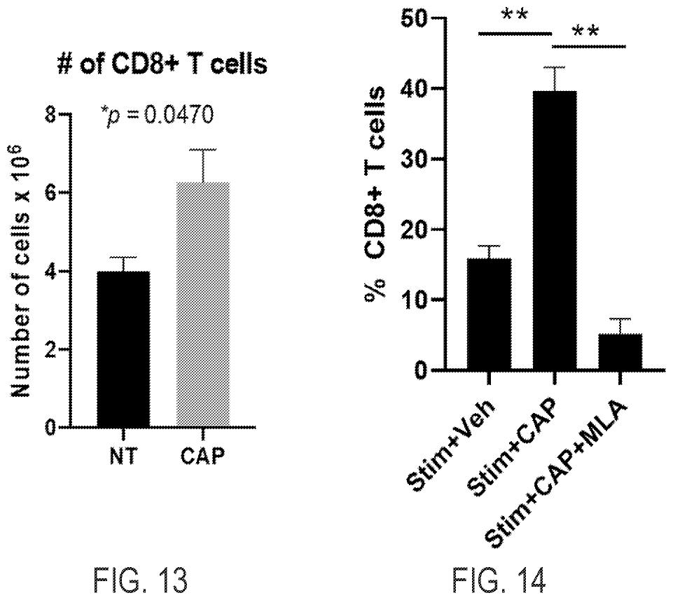

The effects of CAP on viral CD8+ T cells were assessed to demonstrate the effectiveness of the peptide therapy in the treatment of viral infection associated with significant inflammatory responses, such as SARS CoV-2. These T cells are vital for recognition, immobilization and clearance of viral invaders. First, it was demonstrated that CAP treatment is able to induce the expansion of CD8+ T cells (FIG. 13). Next, it was demonstrated that the ability of CAP to induce the expansion of CD8+ T cells was blocked by the α7nAChR antagonist, MCA (FIG. 14).

Collectively, these data suggest that CAP is acting, at least in part, via the α7nAChR in the spleen. The α7nAChR in the spleen is extremely important because it is what transduces the signal of the cholinergic anti-inflammatory pathway, which is a primary mechanism by which the brain exerts control over the inflammatory response.

### Example 6

Preeclampsia

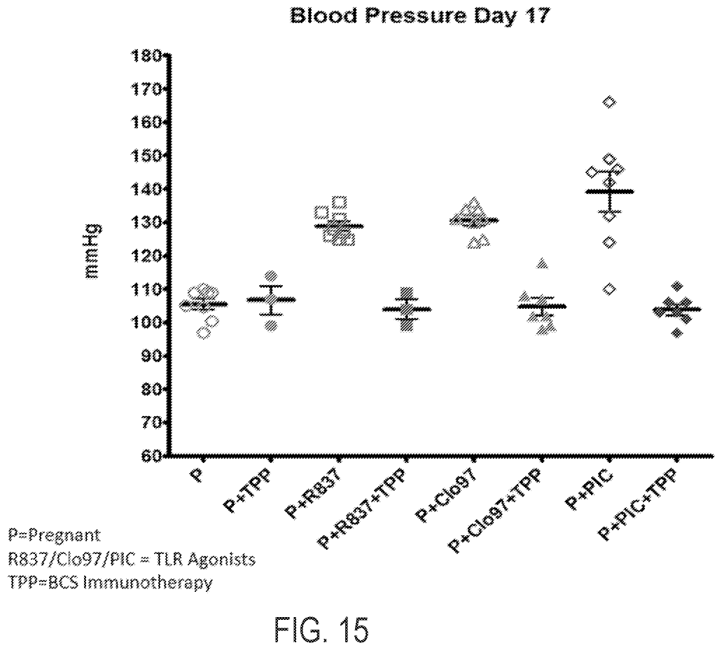

Preeclampsia, a disease suffered by pregnant woman that is characterized by high blood pressure and signs of damage to other organ systems, most often the liver and kidneys, is often accompanied by inflammation. TLR3 and TLR7/8 have both been shown to contribute to preeclampsia in humans and in mice. Using an established model of preeclampsia that is induced in pregnant mice by injection of either the TLR3 agonist, PolyI:C, or with TLR7/8 agonist R83726,30, we demonstrated that CAP (i.p. 2 mg/kg) reduced the elevated blood pressure in this model (FIG. 15A). Preeclampsia is also associated with an elevated TH17 response, including elevated IL17a, and elevated IL17a is associated with human and experimental hypertension and poor perinatal outcomes. IL17a is elevated in our model of preeclampsia (FIG. 15B) and on administration of CAP (i.p. 2 mg/kg) to the pregnant dam was significantly reduced when injected on gestational day 13 (FIG. 15B). Therefore, the anti-inflammatory effects of CAP were associated with improved outcomes in this model.

Sepsis

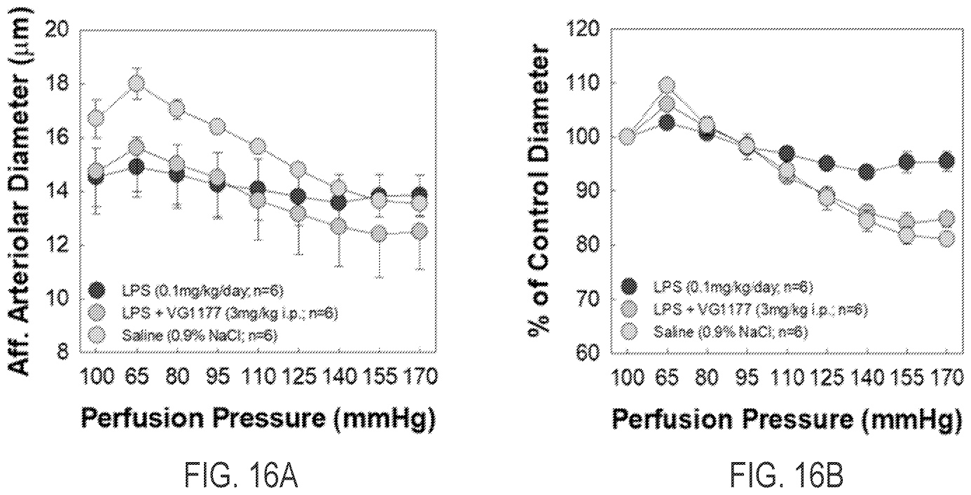

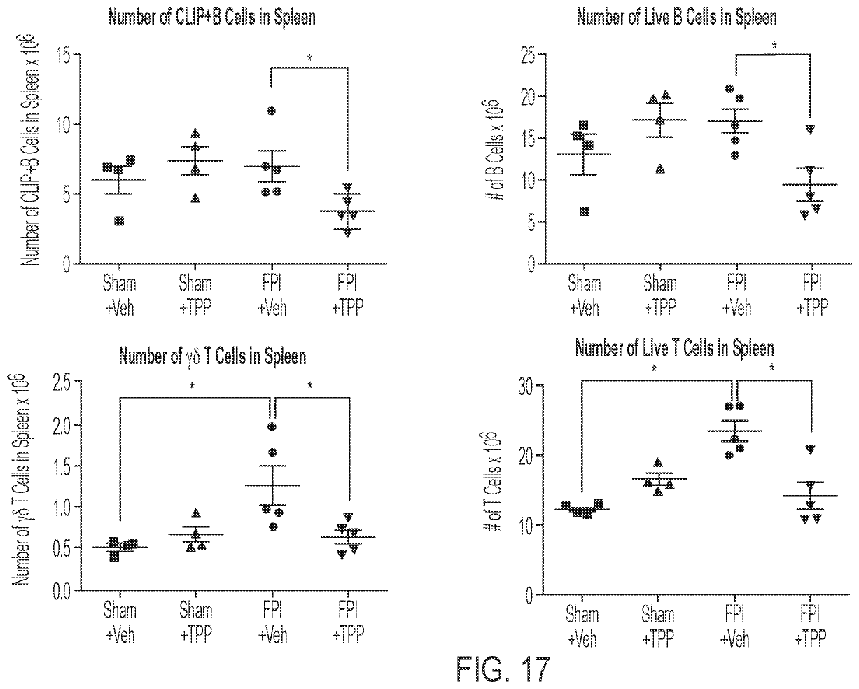

Sepsis is a potentially life-threatening condition, is caused by excessive inflammation resulting from the body trying to fend off infection. Sepsis has been observed to occur in late stage, severe COVID-19 and is associated with loss of arterial pressure and kidney failure. LPS treatment (0.84 mg/kg) of rats is used as a model of sepsis and results in loss of arterial pressure that is inflammatory and mimics loss of autoregulation in sepsis. We discovered that autoregulation of arterial pressure can be restored with CAP treatment (FIG. 16), further supporting its use to treat infectious disease associated with sepsis.

Rheumatoid Arthritis

Rheumatoid arthritis (RA), an autoimmune condition caused by the immune system attacking healthy body tissue by directing antibodies to the lining of the joints, where they attack the tissue surrounding the joint. In an established mouse model of RA, induced by injection of collagen and Complete Freunds Adjuvant (CFA), there is an increase in the pro-inflammatory cytokines, IL1B and IL6 (FIG. 17). In this RA model, these increases were observed in both the developing and established stages of arthritis (FIG. 17). Importantly. treatment with CAP (i.p. 2 mg/kg) during the developing stage was most efficacious at reducing IL1B and IL6, whereas treatment with CAP (20 mg/kg; i.p.) was most efficacious at reducing IL1B and IL6 during the established stage (FIG. 17) of arthritic disease. Therefore, different doses of CAP appear to be effective in the developing versus the established stages of the disease. These changes were accompanied by a reduction in peripheral B cells. These findings are important because IL6 and IL1B are differentially elevated depending on the severity of various infections, such as SARS-CoV-2. Thus, testing different doses of CAP at different stages of infection will be helpful to optimizing translational therapy.

Additional embodiments of the invention include:

Embodiment 1. A method for reducing adverse effects of an immune therapy in a subject being treated with the immune therapy, comprising,

systemically administering to a subject receiving an immune therapy an isolated therapeutic compound, optionally an isolated therapeutic peptide, and a small molecule nicotinic acetylcholine (α7nACh) Receptor agonist, wherein the isolated therapeutic compound and α7nACh receptor agonist are administered in an effective amount to reduce or eliminate a cytokine storm caused by the immune therapy, wherein the immune therapy is a checkpoint inhibitor therapy or cell therapy such as CAR-T cell therapy.

Embodiment 2. The method of Embodiment 1, wherein the subject has cancer.

Embodiment 3. The method of Embodiment 2, further comprising administering to the subject a checkpoint inhibitor.

Embodiment 4. The method of Embodiment 2, wherein the subject has a melanoma.

Embodiment 5. The method of Embodiment 3, wherein the checkpoint inhibitor is an antibody selected from an anti-CTLA4 antibody or antigen-binding fragment thereof that specifically binds CTLA4, an anti-PD1 antibody or antigen-binding fragment thereof that specifically binds PD1, an anti-PD-L1 antibody or antigen-binding fragment thereof that specifically binds PD-L1, an antibody selected from an anti-VISTA antibody or antigen-binding fragment thereof that specifically binds VISTA and a combination thereof.

Embodiment 6. The method of Embodiment 3 wherein the checkpoint inhibitor is an anti-PD-L1 antibody selected from atezolizumab, avelumab, or durvalumab.

Embodiment 7. The method of Embodiment 3, wherein the checkpoint inhibitor is an anti-CTLA-4 antibody selected from tremelimumab or ipilimumab.

Embodiment 8. The method of Embodiment 3, wherein the checkpoint inhibitor is an anti-PD1 antibody selected from nivolumab or pembrolizumab.

Embodiment 9. A kit comprising

a container housing a therapeutic peptide, wherein the therapeutic peptide is a nicotinic acetylcholine (α7nACh) receptor agonist and a CLIP inhibitor,

a container housing a small molecule α7nACh receptor agonist,

and instructions for administering the combination of the therapeutic peptide and the small molecule to a subject in need thereof.

Embodiment 10. A method, comprising

administering to a subject receiving an organ transplant from a donor an isolated therapeutic compound, optionally an isolated therapeutic peptide, wherein the isolated therapeutic compound a donor specific CLIP inhibitor in an effective amount to suppress an immune response in the subject to the donor organ.

Embodiment 11. The method of Embodiment 10, further comprising administering a small molecule nicotinic acetylcholine (α7nACh) receptor agonist to the subject.

Embodiment 12. A method for treating a disorder, comprising

administering to the subject an isolated selective α7 nicotinic acetylcholine receptor (alpha 7 nAChR) agonist in an effective amount to treat the disorder, wherein the selective α7 nAChR agonist is a therapeutic peptide comprising ANSGZ₁I Z₂LA Z₃GGQY (SEQ ID NO: 379), wherein Z₁ and Z₂ are each, independently, two to five amino acids, and wherein Z₃ is one to two amino acids.

Embodiment 13. The method of Embodiment 12, wherein the disorder is cancer.

Embodiment 14. The method of Embodiment 12, wherein the disorder is Alzheimer's disease.

Embodiment 15. The method of Embodiment 12, wherein the disorder is multiple sclerosis.

Embodiment 16. The method of Embodiment 12, wherein the disorder is disorder associated with a hyper-immune response.

Embodiment 17. The method of Embodiment 16, wherein the disorder associated with a hyper-immune response is an infectious disease.

Embodiment 18. The method of Embodiment 17, wherein the infectious disease is an Ebola, SARS, SARS-CoV-2, or MERS, Streptococcus, Staphylococcus, Coronaviruses, or Hantaviruses infection.

Embodiment 19. A method for reducing adverse effects of an immune therapy in a subject being treated with the immune therapy, comprising,

systemically administering to a subject receiving an immune therapy a selective α7 nicotinic acetylcholine receptor (alpha 7 nAChR) agonist in an effective amount to reduce or eliminate a cytokine storm caused by the immune therapy, wherein the α7 nAChR agonist is a therapeutic peptide comprising ANSGZ₁I Z₂LA Z₃GGQY (SEQ ID NO: 379), wherein Z₁ and Z₂ are each, independently, two to five amino acids, and wherein Z₃ is one to two amino acids, wherein the immune therapy is a checkpoint inhibitor therapy or cell therapy such as CAR-T cell therapy.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A therapeutic peptide comprising:
ANSGZ₁I Z₂LA Z₃GGQY (SEQ ID NO: 379), wherein Z₁ and Z₂ are each, independently, two to five amino acids, and wherein Z₃ is one to two amino acids.

2. The peptide of claim 1, wherein Z₁ is two amino acids.

3. The peptide of claim 1, wherein Z₁ is X₁₁₂, wherein X₁ is an amino acid selected from the group consisting of I (Isoleucine) and F (Phenylalanine) and R is Arginine.

4. The peptide of any one of claims 1-3, wherein Z₂ is two amino acids.

5. The peptide of any one of claims 1-4, wherein Z₂ is MAX₂, wherein X₂ is an amino acid selected from the group consisting of T (Threonine) and V (Valine), M is Methionine and A is Alanine.

6. The peptide of any one of claims 1-5, wherein Z₃ is one amino acid.

7. The peptide of any one of claims 1-6, wherein Z₃ is an amino acid selected from the group consisting of I (Isoleucine) and S (Serine).

8. The peptide of any one of claims 1-7, wherein the peptide comprises
(SEQ ID NO: 380)
ANSGIRIMATLAIGGQY.

9. The peptide of any one of claims 1-7, wherein the peptide consists essentially of
(SEQ ID NO: 380)
ANSGIRIMATLAIGGQY.

10. The peptide of any one of claims 1-7, wherein the peptide comprises
(SEQ ID NO: 381)
ANSGFRIMAVLAIGGQY.

11. The peptide of any one of claims 1-7, wherein the peptide consists essentially of
(SEQ ID NO: 381)
ANSGFRIMAVLAIGGQY.

12. The peptide of any one of claims 1-7, wherein the peptide comprises
(SEQ ID NO: 382)
ANSGIRIMAVLASGGQY.

13. The peptide of any one of claims 1-7, wherein the peptide consists essentially of
(SEQ ID NO: 382)
ANSGIRIMAVLASGGQY.

14. The peptide of claim 1, wherein Z₁ is five amino acids.

15. The peptide of claim 1, wherein Z₁ is LENLV (SEQ ID NO: 385), wherein L is Leucine, E is Glutamate, N is Asparagine and V is Valine.

16. The peptide of any one of claims 14-15, wherein Z₂ is five amino acids.

17. The peptide of any one of claims 14-16, wherein Z₂ is LNAAS (SEQ ID NO: 386), wherein L is Leucine, N is Asparagine, A is Alanine and S is Serine.

18. The peptide of any one of claims 14-17, wherein Z₃ is two amino acids.

19. The peptide of any one of claims 14-18, wherein Z₃ is GT, wherein G is Glycine and T is Threonine.

20. The peptide of claim 1, wherein the peptide comprises
(SEQ ID NO: 387)
ANSGLENLVILNAASLAGTGGQY.

21. The peptide of any one of claims 1-7, wherein the peptide consists essentially of
(SEQ ID NO: 387)
ANSGLENLVILNAASLAGTGGQY.

22. A composition comprising the peptide of any one of claims 1-21 and a pharmaceutically acceptable carrier.

23. A method of treating a subject having a disorder associated with blood brain barrier (BBB) permeability, comprising
identifying a subject having a disorder associated with BBB permeability, and administering to the subject an isolated selective α7 nicotinic acetylcholine receptor (α7nAChR) agonist in an effective amount to treat the disorder associated with BBB, wherein the selective α7 nAChR agonist is an isolated therapeutic peptide.

24. The method of claim 23, wherein the isolated therapeutic peptide comprises ANSGZ₁I Z₂LA Z₃GGQY (SEQ ID NO: 379), wherein Z₁ and Z₂ are each, independently, two to five amino acids, and wherein Z₃ is one to two amino acids.

25. The method of claim 24, wherein the isolated therapeutic compound is an isolated therapeutic peptide which is a peptide of any one of claims 2-21.

26. The method of claim 23, wherein the isolated therapeutic peptide comprises an isolated peptide comprising X₁RX₂X₃X₄X₅LX₆X₇ (SEQ ID NO: 383), wherein each X is an amino acid, wherein R is Arginine, L is Leucine and wherein at least one of X₂ and X₃ is Methionine.

27. The method of claim 26, wherein X₁ is Phenylalanine, wherein X₂ is Isoleucine; wherein X₃ is Methionine, wherein X₄ is Alanine, wherein X₅ is Valine, wherein X₆ is Alanine, and wherein X₇ is Serine.

28. The method of claim 26, wherein the peptide comprises FRIMX₄VLX₆S (SEQ ID NO: 388), wherein X₄ and X₆ are any amino acid.

29. The method of claim 26, wherein the peptide comprises FRIMAVLAS (SEQ ID NO: 389).

30. The method of claim 26, wherein the peptide has 9-20 amino acids.

31. The method of claim 26, wherein the peptide is non-cyclic.

32. The method of any one of claims 23-31, wherein a TLR agonist is administered to the subject, wherein the TLR agonist is optionally a TLR 1, 2, 3, 4, 5, 6, 7, 8, TLR agonist or a TLR9 agonist and wherein the TLR agonist is optionally selected from CpG oligonucleotides and pathogen associated molecular patterns (PAMPs), including products of bacteria, viruses, parasites, flagellum, and fungi.

33. The method of any one of claims 23-32, further comprising administering a small molecule α7nACh Receptor agonist to the subject.

34. The method of any one of claims 23-31, wherein the subject is exposed to an environmental stimulus of TLR activity.

35. The method of claim 34, wherein the environmental stimulus of TLR activity is an injury to an organ of the subject.

36. The method of claim 35, wherein the organ of the subject is a brain.

37. The method of claim 34, wherein the environmental stimulus of TLR activity is environmental particulate pollution.

38. The method of claim 37 wherein the environmental particulate pollution is environmental particulates that are rich in non-viable microbial fragments or by-products.

39. A method for reducing adverse effects of an immune response in a subject having a hyper-immune response, comprising
systemically administering to the subject an isolated therapeutic compound comprising a selective α7 nicotinic acetylcholine receptor (α7nAChR) agonist in an effective amount to reduce or eliminate a cytokine storm in the subject, wherein the α7 nAChR agonist is a therapeutic peptide comprising ANSGZ₁I Z₂LA Z₃GGQY (SEQ ID NO: 379), wherein Z₁ and Z₂ are each, independently, two to five amino acids, and wherein Z₃ is one to two amino acids.

40. A method for reducing adverse effects of an immune therapy in a subject having a hyper-immune response, comprising
systemically administering to the subject an isolated therapeutic peptide and a small molecule nicotinic acetylcholine (α7nACh) Receptor agonist, wherein the isolated therapeutic peptide and α7nACh receptor agonist are administered in an effective amount to reduce or eliminate a cytokine storm.

41. The method of claim 39 or 40, wherein the subject has an infectious disease.

42. The method of claim 41, wherein the subject is administered the isolated therapeutic compound or peptide in an effective amount to reduce or eliminate a cytokine storm caused by an infectious agent causing the disease.

43. The method of claim 42, wherein the infectious agent is Ebola, SARS, SARS-CoV-2, or MERS.

44. The method of claim 42, wherein the infectious agent is Streptococcus, Staphylococcus, Coronaviruses, or Hantaviruses.

45. The method of claim 42, wherein the subject has a post-infectious chronic inflammatory syndrome.

46. A method for treating a subject having a post-infectious chronic inflammatory syndrome, comprising
identifying a subject having a post-infectious chronic inflammatory syndrome and systemically administering to the subject an isolated therapeutic peptide in an effective amount to treat a post-infectious chronic inflammatory syndrome.

47. A method for treating a disorder, comprising
administering to a subject having the disorder an isolated therapeutic compound, optionally an isolated therapeutic peptide, wherein the isolated therapeutic compound is a nicotinic acetylcholine (α7nACh) receptor agonist and a CLIP inhibitor, in an effective amount to treat the disorder.

48. The method of claim 47, wherein the disorder is a psychiatric or neurological disease.

49. The method of claim 48, wherein the psychiatric or neurological disease is selected from the group consisting of attention deficit hyperactivity disorder (ADHD), Parkinson's Disease, PANS, PANDAS, Huntington's chorea, epilepsy, convulsions, Tourette syndrome, obsessive compulsive disorder (OCD), memory deficits and dysfunction, a learning deficit, a panic disorder, narcolepsy, nociception, autism, tardive dyskinesia, social phobia, pseudo dementia neuropathic pain, postoperative pain, inflammatory pain, and phantom limb pain.

50. The method of claim 48, wherein the psychiatric or neurological disease is selected from the group consisting of schizophrenia, mania, depression, and anxiety

51. The method of claim 48, wherein the psychiatric or neurological disease is a neurodegenerative disorder selected from the group consisting of: senile dementia and an intellectual impairment disorder.

52. The method of claim 47, wherein the disorder is a damaged tissue and the method is a method of promoting wound healing of a chronic wound.

53. The method of claim 53, wherein the disorder is an imbalance and the method is a method for improving cognition or cessation of addiction.

54. The method of claim 50, wherein the addiction is smoking, vaping, alcohol and/or drugs.

55. The method of claim 47, wherein the disorder is a proliferative and non-neuronal immune disorders.

56. The method of claim 55, wherein the proliferative and non-neuronal immune disorders is selected from the group consisting of autoimmune disease, Inflammatory Bowel Disease, Crohn's disease, asthma, macular degeneration (e.g., dry AMD, wet AMD), retinopathy (e.g., diabetic retinopathy), kidney disease, preeclampsia, type 1 diabetes, arthritis (e.g., osteoarthritis, rheumatoid arthritis (RA), psoriatic arthritis and sepsis.

57. The method of any one of claims 47-55, wherein the isolated therapeutic peptide comprises ANSGZ₁I Z₂LA Z₃GGQY (SEQ ID NO: 379), wherein Z₁ and Z₂ are each, independently, two to five amino acids, and wherein Z₃ is one to two amino acids.

58. The method of claim 57, wherein the isolated therapeutic compound is an isolated therapeutic peptide which is a peptide of any one of claims 2-21.

59. The method of any one of claims 47-55, wherein the isolated therapeutic peptide comprises an isolated peptide comprising X₁RX₂X₃X₄X₅LX₆X₇ (SEQ ID NO: 383), wherein each X is an amino acid, wherein R is Arginine, L is Leucine and wherein at least one of X₂ and X₃ is Methionine.

60. The method of claim 59, wherein X₁ is Phenylalanine, wherein X₂ is Isoleucine; wherein X₃ is Methionine, wherein X₄ is Alanine, wherein X₅ is Valine, wherein X₆ is Alanine, and wherein X₇ is Serine.

61. The method of claim 59, wherein the peptide comprises FRIMX₄VLX₆S, wherein X₄ and X₆ are any amino acid (SEQ ID NO: 388).

62. The method of claim 59, wherein the peptide comprises FRIMAVLAS (SEQ ID NO: 389).

63. The method of claim 59, wherein the peptide has 9-20 amino acids.

64. The method of claim 59, wherein the peptide is non-cyclic.

65. The method of any one of claims 47-64, wherein a TLR agonist is administered to the subject, wherein the TLR agonist is optionally a TLR 1, 2, 3, 4, 5, 6, 7, 8, TLR agonist or a TLR9 agonist and wherein the TLR agonist is optionally selected from CpG oligonucleotides and pathogen associated molecular patterns (PAMPs), including products of bacteria, viruses, parasites, flagellum, and fungi.

66. The method of any one of claims 47-65, further comprising administering a small molecule α7nACh Receptor agonist to the subject.

67. The method of any one of claims 47-65, wherein the subject is exposed to an environmental stimulus of TLR activity.

68. The method of claim 67, wherein the environmental stimulus of TLR activity is an injury to an organ of the subject.

69. The method of claim 68, wherein the organ of the subject is a brain.

70. The method of claim 67, wherein the environmental stimulus of TLR activity is environmental particulate pollution.

71. The method of claim 70, wherein the environmental particulate pollution is environmental particulates that are rich in non-viable microbial fragments or by-products.

72. A method for treating a disorder, comprising administering to the subject an isolated therapeutic compound, optionally an isolated therapeutic peptide and a small molecule nicotinic acetylcholine (α7nACh) receptor agonist, wherein the isolated therapeutic compound a α7nACh receptor agonist and a CLIP inhibitor in an effective amount to treat the disorder.
